(19) United States
(12) Patent Application Publication
(10) Pub. No.: US 2004/0241737 A1
(43) Pub. Date: Dec. 2, 2004

(54) Methods for diagnosing mood disorders

(75) Inventors: Xavier Zafar Khawaja, Yardley, PA (US); Jun Xu, Monmouth Junction, NJ (US); Jinjun Liang, Princeton, NJ (US)

Correspondence Address: WYETH, PATENT LAW GROUP, 5 GIRALDA FARMS, MADISON, NJ, 07940, US

(73) Assignee: Wyeth, Madison, NJ

(21) Appl. No.: 10/851,921

(22) Filed: May 21, 2004

Related U.S. Application Data
(60) Provisional application No. 60/473,625, filed on May 27, 2003.

(51) Int. Cl. C12Q 1/68

(52) U.S. Cl. 435/6

(57) ABSTRACT

The present invention relates generally to the fields of neuroscience, proteomics and mood disorders. More particularly, the present invention relates to the identification of a group of proteins modulated in subjects with a mood disorder; methods for detecting or screening mRNA encoding these proteins and methods for diagnosing mood disorders.

This application claims the benefit under 35 U.S.C. §119(e) to U.S. provisional application No. 60/473,625, filed May 27, 2003, which is hereby incorporated by reference in its entirety.

### FIELD OF THE INVENTION

The present invention relates generally to the fields of neuroscience, proteomics and mood disorders. More particularly, the invention relates to the identification of a group of proteins which are modulated in subjects with a mood disorder, methods for detecting or screening mRNA encoding these proteins and methods for diagnosing mood disorders.

### BACKGROUND OF THE INVENTION

Depression is one of the most prevalent and costly brain diseases (Nemeroff and Owens, 2002), and has been estimated to be the second leading cause of disability worldwide, surpassed only by ischemic heart disease (Murray and Lopez, 1996). For example, in the last major epidemiology study conducted in the United States, major depression had an overall lifetime prevalence rate of 17.1% (21% in women and 13% in men), and comparable figures have been obtained worldwide (Kessler et al., 1994). These findings represent an increase in depression of approximately 6% in the 15 years since the previous study (Blazer et al., 1994), which does not include another 1.3-1.8% of the population afflicted with bipolar disorder.

Moreover, depression is often associated with comorbid psychiatric mood disorders, most notably anxiety disorders (e.g., panic disorder, generalized anxiety disorder, social anxiety disorder, obsessive-compulsive disorder and post-traumatic stress disorder). The mean age of onset of depression has markedly decreased from the 40- to 50-year-old range noted several years ago to the 25- to 35-year range, and this phenomenon has been observed worldwide (Klerman et al., 1985).

The onset of a depressive disorder typically involves a combination of genetic, psychological and environmental factors, after which, later episodes of the illness (i.e., the depressive disorder) are typically precipitated by mild stress stimuli. Clinical depression is most likely a multi-step process in which the delicate homeostatic balance between neurotransmitters and/or hormonal levels and cellular signaling processes become perturbed in the central nervous system (CNS) due to genetic and non-genetic alterations, which may result in structural changes in the brain (Manji et al., 2001; Nestler et al., 2002).

Imbalances in monoamine levels and subsequent loss in postsynaptic signaling mechanisms and/or a stress-induced impairment in the adreno-hypothalamo-pituitary axis have formed the basis for a diverse range of on-going strategies for the treatment of depression and related disorders. Both fluoxetine (a selective serotonin re-uptake inhibitor) and venlafaxine (a dual norepinephrine and serotonin re-uptake inhibitor) represent newer medications with proven clinical efficacy and generally fewer side effects than tricyclics. Although biochemical and pharmacological studies in rodents suggest an acute effect of both drugs to elevate serotonin (5-HT) and norepinephrine (NE) neurotransmitters at postsynaptic junctions (especially in frontal cortex and hippocampus), the onset of a measurable antidepressant effect in the clinic typically requires two to six weeks post-treatment. This has led to the suggestion that long-term, centrally-mediated neuroadaptive changes are required prior to therapeutic efficacy.

The diverse range of existing antidepressant therapies drive cellular responses to a variety of signals, including neurotransmitters, depolarization, synaptic activity, mitogenic and differentiating factors, and stressors (Frazer, 1997). However, the many structural and signaling components leading to antidepressant-mediated adaptive changes in the hippocampus and other areas of the brain remain poorly understood. Stress and major depression inhibit adult hippocampal neurogenesis, possibly via the associated reductions in serotonin or increases in circulating glucocorticoids (Kempermann, 2002). Chronic administration of several classes of antidepressant, including fluoxetine, was reported to increase the proliferation of BrdU-positive cell numbers in the dentate gyrus and hilus of the hippocampus, suggesting that this biological function may be a common and selective action of antidepressants (Malberg et al., 2000). The rates of neuronal proliferation, differentiation, and survival of dentate granule neurons and the length and arborization of apical dendrites of CA3 neurons are influenced by a number of physiological and environmental conditions, for which many of the molecular components and regulatory influence of antidepressants have yet to be fully elucidated.

Depression often goes undetected, especially in children, adolescents and the elderly. Mood disorders are associated with a significant risk for suicide, which remains one of the top ten causes of death in the United States and in many countries throughout the world. Depression is a major independent risk factor for the development of coronary artery disease and stroke, and possibly other major medical disorders. The presence of depression after myocardial infarction is associated with a markedly diminished survival rate over the 18 months after the initial episode. The precise pathophysiology of mood disorders remains obscure, as does the neurobiology of normal mood regulation.

Accordingly, there is a need in the art for methods which identify the structural and/or signaling components which lead to changes in the brain, particularly the hippocampus, of subjects having mood disorders such as bipolar depressive disorder, chronic major depressive disorder and the like. Similarly, there is a need in the art for the early detection, screening and diagnosis of individuals at risk for a mood disorder.

### SUMMARY OF THE INVENTION

The present invention is directed to the identification of a group of proteins which are modulated in subjects treated with monoamine re-uptake inhibitors, methods for detecting or screening these proteins, methods for detecting or screening mRNA encoding these proteins and methods for diagnosing or detecting individuals at risk for mood disorders.

More particularly, in certain embodiments, the invention is directed to a method of screening for a mood disorder in a human subject comprising the steps of obtaining a biological sample from the subject; contacting the sample with a polynucleotide probe complementary to an insulin-like growth factor 1 (IGF-1) mRNA; measuring the amount of probe bound to the mRNA and comparing the amount of bound probe with IGF-1 mRNA in human samples obtained from a statistically significant population lacking the mood disorder, wherein lower IGF-1 mRNA levels in the subject indicates a predisposition to the mood disorder. In certain embodiments, the probe complementary to an IGF-1 mRNA comprises a nucleotide sequence which hybridizes under high stringency hybridization conditions with a polynucleotide comprising the nucleotide sequence of SEQ ID NO:1.

In another embodiment, the invention is directed to a method of screening for a mood disorder in a human subject comprising the steps of obtaining a biological sample from the subject; contacting the sample with a polynucleotide probe complementary to a GMF-β mRNA; measuring the amount of probe bound to the mRNA and comparing the amount of bound probe with GMF-β mRNA in human samples obtained from a statistically significant population lacking the mood disorder, wherein lower GMF-β mRNA levels in the subject indicates a predisposition to the mood disorder. In certain embodiments, the probe complementary to a GMF-β mRNA comprises a nucleotide sequence which hybridizes under high stringency hybridization conditions with a polynucleotide comprising the nucleotide sequence of SEQ ID NO:3.

In another embodiment, the invention is directed to a method of screening for a mood disorder in a human subject comprising the steps of obtaining a biological sample from the subject; contacting the sample with a polynucleotide probe complementary to a collapsin response mediator protein 2 (CRMP2) mRNA; measuring the amount of probe bound to the mRNA and comparing the amount of bound probe with CRMP2 mRNA in human samples obtained from a statistically significant population lacking the mood disorder, wherein lower CRMP2 mRNA levels in the subject indicates a predisposition to the mood disorder. In certain embodiments, the probe complementary to a CRMP2 mRNA comprises a nucleotide sequence which hybridizes under high stringency hybridization conditions with a polynucleotide comprising the nucleotide sequence of SEQ ID NO:5.

In yet another embodiment, the invention is directed to a method of screening for a mood disorder in a human subject comprising the steps of obtaining a biological sample from the subject; contacting the sample with a polynucleotide probe complementary to a PCTAIRE-3 mRNA; measuring the amount of probe bound to the mRNA and comparing the amount of bound probe with PCTAIRE-3 mRNA in human samples obtained from a statistically significant population lacking the mood disorder, wherein lower PCTAIRE-3 mRNA levels in the subject indicates a predisposition to the mood disorder. In certain embodiments, the probe complementary to a PCTAIRE-3 mRNA comprises a nucleotide sequence which hybridizes under high stringency hybridization conditions with a polynucleotide comprising the nucleotide sequence of SEQ ID NO:7.

In other embodiments, the invention is directed to a method of screening for a mood disorder in a human subject comprising the steps of obtaining a biological sample from the subject; contacting the sample with a polynucleotide probe complementary to a HCNP precursor protein mRNA; measuring the amount of bound probe to the mRNA and comparing the amount of bound probe with HCNP precursor protein mRNA in human samples obtained from a statistically significant population lacking the mood disorder, wherein lower HCNP precursor protein mRNA levels in the subject indicates a predisposition to the mood disorder. In certain embodiments, the probe complementary to a HCNP mRNA comprises a nucleotide sequence which hybridizes under high stringency hybridization conditions with a polynucleotide comprising the nucleotide sequence of SEQ ID NO:9.

In yet another embodiment, the invention is directed to a method of screening for a mood disorder in a human subject comprising the steps of obtaining a biological sample from the subject; contacting the sample with a polynucleotide probe complementary to a hydroxysteroid sulfotransferase mRNA; measuring the amount of probe bound to the mRNA and comparing the amount of bound probe with hydroxysteroid sulfotransferase mRNA in human samples obtained from a statistically significant population lacking the mood disorder, wherein lower hydroxysteroid sulfotransferase mRNA levels in the subject indicates a predisposition to the mood disorder. In certain embodiments, the probe complementary to a hydroxysteroid sulfotransferase mRNA comprises a nucleotide sequence which hybridizes under high stringency hybridization conditions with a polynucleotide comprising the nucleotide sequence of SEQ ID NO:11.

In certain other embodiments, the invention is directed to a method of screening for a mood disorder in a human subject comprising the steps of obtaining a biological sample from the subject; contacting the sample with a polynucleotide probe complementary to a pyruvate dehydrogenase-E1 mRNA; measuring the amount of bound probe to the mRNA and comparing the amount of bound probe with pyruvate dehydrogenase-E1 mRNA in human samples obtained from a statistically significant population lacking the mood disorder, wherein lower pyruvate dehydrogenase-E1 mRNA levels in the subject indicates a predisposition to the mood disorder. In certain embodiments, the probe complementary to a pyruvate dehydrogenase-E1 mRNA comprises a nucleotide sequence which hybridizes under high stringency hybridization conditions with a polynucleotide comprising the nucleotide sequence of SEQ ID NO:13.

In other embodiments, the invention is directed to a method of screening for a mood disorder in a human subject comprising the steps of obtaining a biological sample from the subject; contacting the sample with a polynucleotide probe complementary to an antioxidant protein-2 mRNA; measuring the amount of probe bound to the mRNA and comparing the amount of bound probe with antioxidant protein-2 mRNA in human samples obtained from a statistically significant population lacking the mood disorder, wherein lower antioxidant protein-2 mRNA levels in the subject indicates a predisposition to the mood disorder. In certain embodiments, the probe complementary to an antioxidant protein-2 mRNA comprises a nucleotide sequence which hybridizes under high stringency hybridization conditions with a polynucleotide comprising the nucleotide sequence of SEQ ID NO:15.

In another embodiment, the invention is directed to a method of screening for a mood disorder in a human subject comprising the steps of obtaining a biological sample from the subject; contacting the sample with a polynucleotide probe complementary to a DDAH-1 mRNA; measuring the amount of probe bound to the mRNA and comparing the amount of bound probe with DDAH-1 mRNA in human samples obtained from a statistically significant population lacking the mood disorder, wherein lower DDAH-1 mRNA levels in the subject indicates a predisposition to the mood disorder. In certain embodiments, the probe complementary to a DDAH-1 mRNA comprises a nucleotide sequence which hybridizes under high stringency hybridization conditions with a polynucleotide comprising the nucleotide sequence of SEQ ID NO:17.

In a particular embodiment, the mood disorder is selected from the group consisting of unipolar depressive disorder, bipolar depressive disorder, anxiety disorder, panic disorder, dysthymic disorder, postpartum depressive disorder, chronic major depressive disorder and double depressive disorder. In another embodiment, the biological sample is obtained as a blood sample, a cerebrospinal fluid (CSF) sample, a saliva sample, a skin biopsy, a brain biopsy or a buccal biopsy. In yet another embodiment, the biological sample is selected from the group consisting of blood plasma, serum, erythrocytes, leukocytes, platelets, lymphocytes, macrophages, fibroblast cells, mast cells, fat cells, epithelial cells, nerve cells, glial cells, Schwann cells and progenitor stem cells.

In other embodiments, the invention is directed to a method of screening for a mood disorder in a human subject comprising obtaining a biological sample from the subject; contacting the sample with a plurality of polynucleotide probes, wherein the probes are complementary to an IGF-1 mRNA, a GMF-β mRNA, a CRMP2 mRNA, a PCTAIRE-3 mRNA, a HCNP mRNA, a hydroxysteroid sulfotransferase mRNA, a pyruvate dehydrogenase mRNA, an antioxidant protein-2 mRNA and a DDAH-1 mRNA; measuring the amount of each probe bound to the mRNA and comparing the amount of each bound probe with IGF-1 mRNA, GMF-β mRNA, CRMP2 mRNA, PCTAIRE-3 mRNA, HCNP mRNA, hydroxysteroid sulfotransferase mRNA, pyruvate dehydrogenase mRNA, antioxidant protein-2 mRNA and DDAH-1 mRNA in human samples obtained from a statistically significant population lacking the mood disorder, wherein lower levels of one or more mRNAs in the subject indicates a predisposition to the mood disorder.

In certain other embodiments, the invention is directed to a method of screening for a mood disorder in a human subject comprising obtaining a biological sample from the subject; contacting the sample with a plurality antibodies, wherein the plurality of antibodies specifically bind an IGF-1 protein, a GMF-β protein, a CRMP2 protein, a PCTAIRE-3 protein, a HCNP protein, a hydroxysteroid sulfotransferase protein, a pyruvate dehydrogenase protein, an antioxidant protein-2 protein and a DDAH-1 protein; measuring the amount of each antibody bound to its respective protein and comparing the amount of each bound antibody with IGF-1, GMF-β, CRMP2, PCTAIRE-3, HCNP, hydroxysteroid sulfotransferase, pyruvate dehydrogenase, antioxidant protein-2 and DDAH-1 protein levels from human samples obtained from a statistically significant population lacking the mood disorder, wherein lower levels of one or more proteins in the subject indicates a predisposition to the mood disorder.

In yet another embodiment, the invention is directed to a method for monitoring the kinetics of an inhibitor of a monoamine re-uptake receptor in a rodent comprising the steps of administering to a plurality of rodents a monoamine re-uptake inhibitor or a placebo; obtaining, at a desired time point, a hippocampus from one of the plurality of rodents administered the monoamine re-uptake inhibitor and a hippocampus from one of the plurality of rodents administered a placebo; determining the amount of one or more proteins in the hippocampus of each animal, wherein the one or more proteins are selected from the group consisting of IGF-1, GMF-β, CRMP2, PCTAIRE-3, HCNP, hydroxysteroid sulfotransferase, pyruvate dehydrogenase, antioxidant protein-2 and DDAH-1, and repeating the above steps, wherein a range of desired time points are gathered from 0 days to about 36 days.

In certain other embodiments, the invention is directed to a method of screening for an inhibitor of a monoamine re-uptake receptor comprising the steps of contacting a mammalian cell with a test compound, and detecting the level of one or more proteins selected from the group consisting of IGF-1, GMF-β, CRMP2, PCTAIRE-3, HCNP, hydroxysteroid sulfotransferase, pyruvate dehydrogenase, antioxidant protein-2 and DDAH-1, wherein an increase in level of the one or more proteins, relative to the level of the one or more proteins in the absence of the test compound, indicates the test compound is an inhibitor of a monoamine re-uptake receptor.

In another embodiment, the invention is directed to a transgenic non-human animal comprising one or more exogenous polynucleotides encoding a protein selected from the group consisting of an IGF-1, a GMF-β, a CRMP2, a PCTAIRE-3, a HCNP, a hydroxysteroid sulfotransferase, a pyruvate dehydrogenase, an antioxidant protein-2 and a DDAH-1 protein.

In yet another embodiment, the invention is directed to a transgenic non-human animal having a functional disruption in one or more genes encoding a protein selected from the group consisting of an IGF-1, a GMF-β, a CRMP2, a PCTAIRE-3, a HCNP, a hydroxysteroid sulfotransferase, a pyruvate dehydrogenase, an antioxidant protein-2 and a DDAH-1 protein. In particular embodiments, the animal is heterozygous for the one or more disruptions.

In certain other embodiments, the invention is directed to a method of screening for a mood disorder in a human subject comprising obtaining a biological sample from the subject; applying the sample to a DNA chip comprising an array of polynucleotides, wherein the array comprises at least a nucleotide sequence encoding an IGF-1 protein, a GMF-β protein, a CRMP2 protein, a PCTAIRE-3 protein, a HCNP protein, a hydroxysteroid sulfotransferase protein, a pyruvate dehydrogenase protein, an antioxidant protein-2 protein and a DDAH-1 protein; measuring the amount of each polynucleotide bound to the array; and comparing the amount of polynucleotide bound with IGF-1, GMF-β, CRMP2, PCTAIRE-3, HCNP, hydroxysteroid sulfotransferase, pyruvate dehydrogenase, antioxidant protein-2 and DDAH-1 polynucleotide levels in human samples obtained from a statistically significant population lacking the mood disorder, wherein lower levels of one or more polynucleotides in the subject indicates a predisposition to the mood disorder.

In another embodiment, the invention is directed to a method for screening for a mood disorder in a human subject comprising obtaining a biological sample from the subject; applying the sample to an array of protein-capture agents, wherein a protein-capture agent on the array can bind an IGF-1 protein, a protein-capture agent on the array can bind GMF-β protein, a protein-capture agent on the array can bind a CRMP2 protein, a protein-capture agent on the array can bind a PCTAIRE-3 protein, a protein-capture agent on the array can bind a human HCNP protein, a protein-capture agent on the array can bind a human hydroxysteroid sulfotransferase protein, a protein-capture agent on the array can bind a human pyruvate dehydrogenase-E1 protein, a protein-capture agent on the array can bind a human antioxidant protein-2 protein, and a protein-capture agent on the array can bind a human DDAH-1 protein; measuring the amount of each bound protein; and comparing the amount of bound protein with an array standard obtained from a statistically significant human population lacking the mood disorder, wherein lower levels of one or more proteins in the subject indicates a predisposition to the mood disorder. In a preferred embodiment, the protein-capture agent is an antibody.

In still other embodiments, the invention is directed to a method for treating a mood disorder in a human subject in need thereof comprising delivering a polynucleotide encoding a wild-type IGF-1 polypeptide, a polynucleotide encoding a wild-type a GMF-β polypeptide, a polynucleotide encoding a wild-type a CRMP2 polypeptide, a polynucleotide encoding a wild-type a PCTAIRE-3 polypeptide, a polynucleotide encoding a wild-type a HCNP polypeptide, a polynucleotide encoding a wild-type a hydroxysteroid sulfotransferase polypeptide, a polynucleotide encoding a wild-type a pyruvate dehydrogenase polypeptide, a polynucleotide encoding a wild-type an antioxidant protein-2 polypeptide or a polynucleotide encoding a wild-type a DDAH-1 polypeptide.

In certain other embodiments, the invention is directed to a method for treating a mood disorder in a human subject in need thereof comprising delivering one or more polynucleotides encoding a wild-type IGF-1 polypeptide, a GMF-β polypeptide, a CRMP2 polypeptide, a PCTAIRE-3 polypeptide, a HCNP polypeptide, a hydroxysteroid sulfotransferase polypeptide, a pyruvate dehydrogenase polypeptide, an antioxidant protein-2 polypeptide and a DDAH-1 polypeptide

Other features and advantages of the invention will be apparent from the following detailed description, from the preferred embodiments thereof, and from the claims.

### BRIEF DESCRIPTION OF THE DRAWINGS

FIGS. 1A and 1B demonstrate the effect of venlafaxine and fluoxetine administration on cell proliferation (FIG. 1A) and long-term survivability (FIG. 1B). The number of BrdU-positive cells in the entire SGZ after BrdU administration is presented (mean±SEM). FIG. 1A shows chronic venlafaxine and fluoxetine administration increased the number of proliferating cells in the SGZ compared with saline-treated control rats (# P<0.01). FIG. 1B shows chronic venlafaxine (# P<0.01) and fluoxetine (*P<0.05) increased the number of surviving cells in the SGZ relative to saline controls 4 weeks after the last BrdU injection. A significant increase in the overall number of surviving cells was observed between venlafaxine versus fluoxetine (# P<0.01). Comparative analyses between treatments were performed using a Tukey HSD post hoc analysis for multiple comparisons.

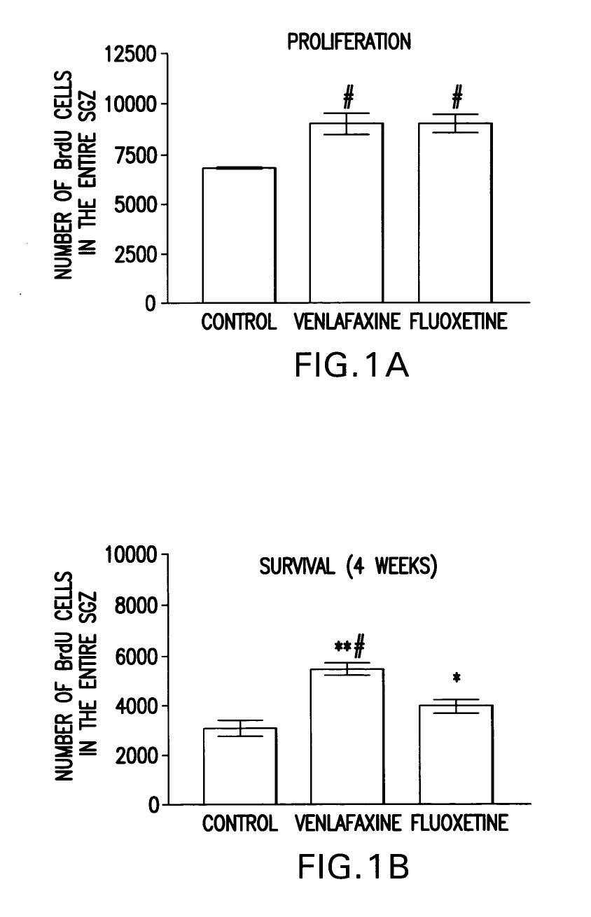

### DETAILED DESCRIPTION OF THE INVENTION

The present invention addresses a need in the art for methods of screening and/or diagnosing mood disorders in humans. Thus, in certain embodiments the invention relates to the identification of a group of proteins which are upregulated in the hippocampus, following antidepressant drug treatment (Table 1). More particularly, a two-dimensional electrophoretic (2-DE) proteome analysis was used to examine the changes occurring in rat brain proteins, following treatment with the monoamine re-uptake inhibitors fluoxetine and venlafaxine. Analysis of the hippocampus was chosen due to the involvement of this important anatomical region in clinical depression (Nestler et al., 2002). A two week post-treatment time-frame was used to characterize the functional role played by this class of antidepressant in the development of long-term neuroadaptive changes. Using this approach, antidepressant-treated versus non-treated rats were compared and showed alterations in the integrated intensity of thirty three protein spots obtained from the soluble protein fraction of rat hippocampal extracts (see, Table 1 and Example 1). Most of the proteins identified were not previously known to be involved in antidepressant-mediated pathways.

The selection criteria for nine of the thirty-three up-regulated proteins (described below and listed in Table 1) was based on intracellular and/or secretory proteins with functional/signaling properties (excluding general housekeeping, metabolic, structural and ribosomal proteins). The nine proteins selected are present in brain and are involved in signaling mechanisms that could be associated with depressive disorders such as synaptic plasticity, neurogenesis and survival. It is contemplated that these nine proteins represent targets for therapeutic compositions (e.g., pharmaceuticals or “drugs”) such as small molecules, mimetics, antibodies, genetic modifications and the like.

Thus, the selected nine up-regulated proteins, listed in Table 1, are Insulin-like growth factor 1 (IGF-1), glia maturation factor β (GMF-β), collapsin response mediator protein 2 (CRMP2), PCTAIRE protein kinase 3 (PCTK3 or PCTAIRE-3), hippocampal cholinergic neurostimulating peptide (HCNP), hydroxysteroid sulfotransferase, pyruvate dehydrogenase-E1, antioxidant protein-2 and dimethylarginine dimethylaminohydrolase 1 (DDAH-1).

TABLE 1
PROTEIN UP-REGULATION IN RAT BRAIN HIPPOCAMPUS FOLLOWING
ANTIDEPRESSANT DRUG TREATMENT

| | | Venlafaxine | Fluoxetine | | Protein |
|---|---|---|---|---|---|
| Original gel | | (Fold | (Fold | MOWSE | Area |
| spot # | Protein Identity | change) | change) | score | Coverage |
| Neurogenic | | | | | |
| involvement | | | | | |
| 87 | IGF-1 A precursor | 2.9 | 2.5 | 7.07E+01 | 28% |
| 67 | GLIA maturation factor beta | 2.7 | 3 | 7.94E+02 | 29% |
| | (GMF-β) | | | | |
| 1 | Dihydropyrimidinase Related | 4.1 | 2.6 | 1.69E+05 | 28% |
| | Protein-2 (DRP-2; CRMP-2; | | | | |
| | TOAD-64). | | | | |
| | Belongs to collapsin response | | | | |
| | mediator protein (CRMP) family. | | | | |
| 6 | PCTAIRE-3 | 1.5 | 1.5 | 1.30E+02 | 19% |
| | (Serine/Threonine-Protein | | | | |
| | Kinase) | | | | |
| 60 | Hippocampal Cholinergic | 2 | 1.9 | 1.11E+04 | 55% |
| | Neurostimulating peptide | | | | |
| | (PEBP; HCNP; Raf kinase | | | | |
| | inhibitor protein) | | | | |
| | A Serine protease inhibitor. | | | | |
| 62 | Serine Protease Inhibitor 2.1 | 1.9 | 2.9 | 2.67E+01 | 21% |
| | (SPI-2.1) | | | | |
| Steroid | | | | | |
| involvement | | | | | |
| 70 | Probasin precursor (lipocalin | 2.7 | 2.7 | 3.52E+01 | 28% |
| | family member) | | | | |
| 79 | Hydroxysteroid | 2 | 2 | 2.46E+02 | 39% |
| | sulfotransferase A | | | | |
| | (Androsterone-sulfating | | | | |
| | sulfotransferase) | | | | |
| Anti-apoptotic | | | | | |
| activity | | | | | |
| 18 | NG, NG-Dimethylarginine | 2.2 | 1.6 | 7.52E+03 | 29% |
| | Dimethylaminohydrolase 1 | | | | |
| | (DDAH1) | | | | |
| Oxidative | | | | | |
| metabolism | | | | | |
| 51 | Antioxidant protein 2 | 1.7 | 1.6 | 2.03E+05 | 50% |
| | (calcium-independent PLA2) | | | | |
| 26 | Pyruvate Dehydrogenase E1 | 2.6 | 3.2 | 1.56E+04 | 23% |
| | component beta subunit, | | | | |
| | mitochondrial (precursor) | | | | |
| Glycolytic | | | | | |
| pathway | | | | | |
| 8 | Alpha-enolase | 2 | 3.4 | 1.28E+06 | 28% |
| 9 | Alpha-enolase | 4.8 | 5 | 1.24E+05 | 26% |
| 10 | Alpha-enolase | 4.1 | 3 | 3.62E+03 | 21% |
| 23 | L-Lactate Dehydrogenase B | 2.5 | 2.5 | 8.19E+03 | 27% |
| | Chain | | | | |
| Vesicular | | | | | |
| transport/ | | | | | |
| chaperones | | | | | |
| 90 (mix) | Ras-related protein RAB-4A | 1.5 | 1.5 | 1.91E+02 | 25% |
| 90 (mix) | Ras-related protein RAB-1B | 1.5 | 1.5 | 4.77E+01 | 22% |
| 83 | 10 KD heat shock protein, | 3.8 | 2.3 | 3.23E+01 | 32% |
| | mitochondrial (Hsp10) | | | | |
| 89 | Fatty Acid-Binding Protein, Heart | 2.1 | 1.6 | 5.47E+01 | 33% |
| | (H-FABP) | | | | |
| Structural | | | | | |
| proteins | | | | | |
| 64 | Myelin Basic Protein S (MBP S) | 3.7 | 3.1 | 1.93E+01 | 24% |
| Chemokine | | | | | |
| pathway | | | | | |
| 80 | Lymphotactin [precursor] | 1.9 | 2 | 2.38E+01 | 35% |
| | (Cytokine SCM-1; Small | | | | |
| | inducible cytokine C1) | | | | |
| 93 | D-dopachrome tautomerase | 2.3 | 2.2 | 7.79E+02 | 36% |
| Purine | | | | | |
| salvage | | | | | |
| pathway | | | | | |
| 57 | Adenine | 3 | 5.8 | 2.32E+02 | 31% |
| | Phosphoribosyltransferase | | | | |
| | (APRT) | | | | |
| Detoxification | | | | | |
| 56 | Glutathione S-Transferase Yb3 | 0.5 | 0.5 | 1.38E+02 | 33% |
| Transcription/ | | | | | |
| translation | | | | | |
| 19 | Transcription factor BTEB 1 | 2.3 | 1.6 | 6.37E+01 | 16% |
| 88 | Transcription Initiation factor IIA | 2.4 | 2.5 | 3.68E+01 | 14% |
| | Gamma Chain | | | | |
| 72 | 40 S ribosomal protein S 19 | 1.8 | 2 | 2.05E+01 | 22% |
| 58 | 60S Ribosomal Protein L18A | 2.9 | 1.8 | 8.21E+01 | 31% |
| 92 | 60S Ribosomal Protein L35A | 2.5 | 2.2 | 1.65E+01 | 27% |
| 66 | 61 S Ribosomal protein L28 | 0.6 | 0.7 | 1.12E+01 | 25% |
| Other | | | | | |
| 27 | Proteasome subunit alpha type 2 | 10.6 | 6.1 | 1.97E+00 | 18% |
| | (Multicatalytic endopeptidase | | | | |
| | complex subunit C3) | | | | |
| 86 | Cytochrome C oxidase | 4 | 3.2 | 1.10E+04 | 37% |
| | polypeptide VB, mitochondrial | | | | |
| | precursor | | | | |
| | (COX5B) | | | | |

A. Isolated Polynucleotides

In certain embodiments, the invention is directed to methods for screening and/or diagnosing mood disorders in human subjects comprising the steps of (1) obtaining a biological sample from a subject; (2) contacting the sample with a polynucleotide probe complementary to one or more mRNA molecules selected from the group consisting of IGF-1 mRNA, GMF-β mRNA, CRMP2 mRNA, PCTAIRE-3 mRNA, HCNP mRNA, hydroxysteroid sulfotransferase mRNA, pyruvate dehydrogenase-E1 mRNA, antioxidant protein-2 mRNA and DDAH-1 mRNA; (3) measuring the amount of probe bound to the mRNA and (4) comparing this amount with the same mRNA molecules in human samples obtained from a statistically significant population lacking the neurological disorder, wherein lower levels of one or more IGF-1 mRNA, GMF-β mRNA, CRMP2 mRNA, PCTAIRE-3 mRNA, HCNP mRNA, hydroxysteroid sulfotransferase mRNA, pyruvate dehydrogenase-E1 mRNA, antioxidant protein-2 mRNA or DDAH-1 mRNA in the subject indicates a predisposition to the neurological disorder.

In still other embodiments, the invention is directed to a method of screening for a mood disorder in a human subject comprising (1) obtaining a biological sample from a human subject; (2) applying the sample to an array of oligonucleotide probes, wherein at least one probe on the array can bind a polynucleotide selected from the group consisting of human IGF-1, human GMF-β, human CRMP2, human PCTAIRE-3, human HCNP, human hydroxysteroid sulfotransferase, human pyruvate dehydrogenase-E1, human antioxidant protein-2 and human DDAH-1; (3) measuring the amount of each polynucleotide bound to its respective oligonucleotide probe; and (4) comparing the level of the bound probe(s) versus an array standard or control obtained from a statistically significant human population lacking the mood disorder.

Thus, in certain embodiments, the present invention provides isolated and purified polynucleotides that are complementary to one or more polynucleotides selected from the group consisting of IGF-1 mRNA, GMF-β mRNA, CRMP2 mRNA, PCTAIRE-3 mRNA, HCNP mRNA, hydroxysteroid sulfotransferase mRNA, pyruvate dehydrogenase-E1 mRNA, antioxidant protein-2 mRNA and DDAH-1 mRNA.

In yet another embodiment, the invention is directed to a method for the treatment of a mood disorder in a human subject in need thereof. Thus, in one particular embodiment, a method for the treatment of a mood disorder comprises delivering one or more polynucleotides encoding one or more wild-type polypeptides selected from the group consisting of human IGF-1, human GMF-β, human CRMP2, human PCTAIRE-3, human HCNP, human hydroxysteroid sulfotransferase, human pyruvate dehydrogenase-E1, human antioxidant protein-2 and human DDAH-1.

In a preferred embodiment, the nucleotide sequence of a human IGF-1 polynucleotide, a human GMF-β polynucleotide, a human CRMP2 polynucleotide, a human PCTAIRE-3 polynucleotide, a human HCNP polynucleotide, a human hydroxysteroid sulfotransferase polynucleotide, a human pyruvate dehydrogenase-E1 polynucleotide, a human antioxidant protein-2 polynucleotide and a human DDAH-1 polynucleotide are set forth in SEQ ID NO:1, SEQ ID NO:3, SEQ ID NO:5, SEQ ID NO:7, SEQ ID NO:9, SEQ ID NO:11, SEQ ID NO:13, SEQ ID NO:15 and SEQ ID NO:17, respectively.

As used herein, the term “polynucleotide” means a sequence of nucleotides connected by phosphodiester linkages. Polynucleotides are presented herein in the direction from the 5′ to the 3′ direction. A polynucleotide of the present invention comprises from about 40 to about several hundred thousand base pairs. Preferably, a polynucleotide comprises from about 10 to about 3,000 base pairs. Preferred lengths of particular polynucleotides are set forth hereinafter.

A polynucleotide of the present invention is a deoxyribonucleic acid (DNA) molecule, a ribonucleic acid (RNA) molecule, or analogs of the DNA or RNA generated using nucleotide analogs. The nucleic acid molecule is single-stranded or double-stranded. Where a polynucleotide is a DNA molecule, that molecule is a gene, a cDNA molecule or a genomic DNA molecule. Nucleotide bases are indicated herein by a single letter code: adenine (A), guanine (G), thymine (T), cytosine (C), inosine (I) and uracil (U).

“Isolated” means altered “by the hand of man” from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide separated from the coexisting materials of its natural state is “isolated,” as the term is defined herein.

Thus, an IGF-1 polynucleotide of the invention is any polynucleotide encoding a human IGF-1 polypeptide having at least about 80%, more preferably at least about 90% and even more preferably at least about 95% sequence identity to an IGF-1 polypeptide of SEQ ID NO:2. A GMF-β polynucleotide of the invention is any polynucleotide encoding a human GMF-β polypeptide having at least about 80%, more preferably at least about 90% and even more preferably at least about 95% sequence identity to a GMF-β polypeptide of SEQ ID NO:4. A CRMP2 polynucleotide of the invention is any polynucleotide encoding a human CRMP2 polypeptide having at least about 80%, more preferably at least about 90% and even more preferably at least about 95% sequence identity to a CRMP2 polypeptide of SEQ ID NO:6. A PCTAIRE-3 polynucleotide of the invention is any polynucleotide encoding a human PCTAIRE-3 polypeptide having at least about 80%, more preferably at least about 90% and even more preferably at least about 95% sequence identity to a PCTAIRE-3 polypeptide of SEQ ID NO:8. A HCNP polynucleotide of the invention is any polynucleotide encoding a human HCNP polypeptide having at least about 80%, more preferably at least about 90% and even more preferably at least about 95% sequence identity to a HCNP polypeptide of SEQ ID NO:10. A hydroxysteroid sulfotransferase polynucleotide of the invention is any polynucleotide encoding a human hydroxysteroid sulfotransferase polypeptide having at least about 80%, more preferably at least about 90% and even more preferably at least about 95% sequence identity to a hydroxysteroid sulfotransferase polypeptide of SEQ ID NO:12. A pyruvate dehydrogenase-E1 polynucleotide of the invention is any polynucleotide encoding a human pyruvate dehydrogenase-E1 polypeptide having at least about 80%, more preferably at least about 90% and even more preferably at least about 95% sequence identity to a pyruvate dehydrogenase-E1 polypeptide of SEQ ID NO:14. An antioxidant protein-2 polynucleotide of the invention is any polynucleotide encoding a human antioxidant protein-2 polypeptide having at least about 80%, more preferably at least about 90% and even more preferably at least about 95% sequence identity to an antioxidant protein-2 polypeptide of SEQ ID NO:16. A DDAH-1polynucleotide of the invention is any polynucleotide encoding a human DDAH-1polypeptide having at least about 80%, more preferably at least about 90% and even more preferably at least about 95% sequence identity to a DDAH-1 polypeptide of SEQ ID NO:18.

Polynucleotides of the present invention are obtained, using standard cloning and screening techniques, from a cDNA library derived from mRNA from human cells or from genomic DNA. Polynucleotides of the invention are also synthesized using well known and commercially available techniques.

In a preferred embodiment, an isolated polynucleotide (or an isolated polynucleotide probe) of the invention comprises a nucleic acid molecule which is a complement of a mRNA molecule having a nucleotide sequence set forth in SEQ ID NO:1, SEQ ID NO:3, SEQ ID NO:5, SEQ ID NO:7, SEQ ID NO:9, SEQ ID NO:11, SEQ ID NO:13, SEQ ID NO:15 or SEQ ID NO:17, or a fragment of one of these nucleotide sequences. A nucleic acid molecule which is complementary to a nucleotide sequence set forth SEQ ID NO:1, SEQ ID NO:3, SEQ ID NO:5, SEQ ID NO:7, SEQ ID NO:9, SEQ ID NO:11, SEQ ID NO:13, SEQ ID NO:15 or SEQ ID NO:17, is one which is sufficiently complementary to the nucleotide sequence, such that it can hybridize to the nucleotide sequence, thereby forming a stable duplex. Examples of hybridization stringency conditions are detailed in Table 2.

Moreover, a polynucleotide of the invention may comprise only a fragment of the coding region of a polynucleotide or gene, such as a fragment of SEQ ID NO:1, SEQ ID NO:3, SEQ ID NO:5, SEQ ID NO:7, SEQ ID NO:9, SEQ ID NO:11, SEQ ID NO:13, SEQ ID NO:15 or SEQ ID NO:17.

In certain embodiments, the polynucleotide sequence information provided by SEQ ID NO:1, SEQ ID NO:3, SEQ ID NO:5, SEQ ID NO:7, SEQ ID NO:9, SEQ ID NO:11, SEQ ID NO:13, SEQ ID NO:15 and SEQ ID NO:17 allows for the preparation of relatively short DNA (or RNA) oligonucleotide sequences having the ability to specifically hybridize to gene, polynucleotide, cDNA or mRNA sequences of the selected polynucleotides disclosed herein. In a preferred embodiment, an oligonucleotide sequence of the invention is one which is complimentary to an IGF-1 polynucleotide, a GMF-β polynucleotide, a CRMP2 polynucleotide, a PCTAIRE-3 polynucleotide, a HCNP polynucleotide, a hydroxysteroid sulfotransferase polynucleotide, a pyruvate dehydrogenase-E1 polynucleotide, an antioxidant protein-2 polynucleotide and/or a DDAH-1 polynucleotide. In another preferred embodiment, an oligonucleotide sequence complimentary to a polynucleotide of SEQ ID NO:1, SEQ ID NO:3, SEQ ID NO:5, SEQ ID NO:7, SEQ ID NO:9, SEQ ID NO:11, SEQ ID NO:13, SEQ ID NO:15 and/or SEQ ID NO:17 is/are used according to the DNA chip (array) methods set forth in Section F.

The term “oligonucleotide” as used herein is defined as a molecule comprised of two or more deoxyribonucleotides or ribonucleotides, usually more than three (3), and typically more than ten (10) and up to one hundred (100) or more. The exact size will depend on many factors, which in turn depends on the ultimate function or use of the oligonucleotide. Thus, in particular embodiments of the invention, nucleic acid probes of an appropriate length are prepared based on a consideration of a selected nucleotide sequence, e.g., a sequence such as that shown in SEQ ID NO:1, SEQ ID NO:3, SEQ ID NO:5, SEQ ID NO:7, SEQ ID NO:9, SEQ ID NO:11, SEQ ID NO:13, SEQ ID NO:15 or SEQ ID NO:17. The ability of such nucleic acid probes to specifically hybridize to a polynucleotide encoding an IGF-1, a GMF-β, a CRMP2, a PCTAIRE-3, a HCNP, a hydroxysteroid sulfotransferase, a pyruvate dehydrogenase-E1, an antioxidant protein-2 or a DDAH-1 polypeptide lends them particular utility in a variety of embodiments. Most importantly, the probes are used in a variety of assays for detecting the presence of complementary sequences in a given sample.

In certain embodiments, it is advantageous to use oligonucleotide primers. These primers are generated in any manner, including chemical synthesis, DNA replication, reverse transcription, or a combination thereof. The sequence of such primers are designed using a polynucleotide of the present invention for use in detecting, amplifying or mutating a defined segment of a gene or polynucleotide that encodes a polypeptide from mammalian cells using polymerase chain reaction (PCR) technology.

In certain embodiments, it is advantageous to employ a polynucleotide of the present invention in combination with an appropriate label for detecting hybrid formation. A wide variety of appropriate labels are known in the art, including radioactive, enzymatic or other ligands, such as avidin/biotin, which are capable of giving a detectable signal.

To provide certain advantages in accordance with the present invention, a preferred nucleic acid sequence employed for hybridization studies or assays includes probe molecules that are complementary to at least a 10 to 70 nucleotide stretch of a polynucleotide that encodes a polypeptide of the invention. A size of at least 10 nucleotides in length helps to ensure that the fragment will be of sufficient length to form a duplex molecule that is both stable and selective. Molecules having complementary sequences over stretches greater than 10 bases in length are generally preferred, though, in order to increase stability and selectivity of the hybrid, and thereby improve the quality and degree of specific hybrid molecules obtained. One will generally prefer to design nucleic acid molecules having gene-complementary stretches of 25 to 40 nucleotides, 55 to 70 nucleotides, or even longer where desired. Such fragments are readily prepared by, for example, directly synthesizing the fragment by chemical means, by application of nucleic acid reproduction technology, such as the PCR technology of U.S. Pat. No. 4,683,202 (incorporated by reference herein in its entirety) or by excising selected DNA fragments from recombinant plasmids containing appropriate inserts and suitable restriction enzyme sites.

Accordingly, a polynucleotide probe molecule of the invention is used for its ability to selectively form duplex molecules with complementary stretches of the gene. Depending on the application envisioned, one will desire to employ varying conditions of hybridization to achieve a varying degree of selectivity of the probe toward the target sequence. For applications requiring a high degree of selectivity, one will typically desire to employ relatively stringent conditions to form the hybrids (see Table 2 below).

A preferred polynucleotide probe for detecting IGF-1, GMF-β, CRMP2, PCTAIRE-3, HCNP, hydroxysteroid sulfotransferase, pyruvate dehydrogenase, antioxidant protein-2 or DDAH-1 mRNA is a labeled or labelable nucleic acid probe capable of hybridizing to IGF-1, GMF-β, CRMP2, PCTAIRE-3, HCNP, hydroxysteroid sulfotransferase, pyruvate dehydrogenase, antioxidant protein-2 or DDAH-1 mRNA. The complementary nucleic acid probe can be, for example, the full-length cDNA, or a fragment thereof, such as an oligonucleotide of at least 15, 30, 50, 100, 250 or 500 nucleotides in length and sufficient to specifically hybridize under stringent conditions to IGF-1, GMF-O, CRMP2, PCTAIRE-3, HCNP, hydroxysteroid sulfotransferase, pyruvate dehydrogenase, antioxidant protein-2 or DDAH-1 mRNA.

The term “biological sample” is intended to include tissues, cells and biological fluids isolated from a subject, as well as tissues, cells and fluids present within a subject. That is, the detection method of the invention can be used to detect IGF-1, GMF-β, CRMP2, PCTAIRE-3, HCNP, hydroxysteroid sulfotransferase, pyruvate dehydrogenase, antioxidant protein-2 or DDAH-1 mRNA (or the encoded protein) in a biological sample in vitro as well as in vivo. For example, in vitro techniques for detection of IGF-1, GMF-β, CRMP2, PCTAIRE-3, HCNP, hydroxysteroid sulfotransferase, pyruvate dehydrogenase, antioxidant protein-2 or DDAH-1 mRNA include Northern hybridizations and in situ hybridizations, include enzyme linked immunosorbent assays (ELISAs), Western blots, immunoprecipitations and immunofluorescence. In vivo techniques for detection include imaging techniques such as magnetic resonance imaging (MRI) or positron emission tomography (PET) scan.

The present invention also includes polynucleotides capable of hybridizing under reduced stringency conditions, more preferably stringent conditions, and most preferably highly stringent conditions, to polynucleotides described herein. Examples of stringency conditions are shown in Table 2 below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 2
HYBRIDIZATION STRINGENCY CONDITIONS

| | Poly- | Hybrid | Hybridization | Wash |
|---|---|---|---|---|
| Stringency | nucleotide | Length | Temperature and | Temperature |
| Condition | Hybrid | (bp)^{I} | Buffer^{H} | and BufferH |
| A | DNA:DNA | >50 | 65° C.; 1xSSC -or- | 65° C.; |
| | | | 42° C.; 1xSSC, 50% | 0.3xSSC |
| | | | formamide | |
| B | DNA:DNA | <50 | T_{B}; 1xSSC | T_{B}; 1xSSC |
| C | DNA:RNA | >50 | 67° C.; 1xSSC -or- | 67° C.; |
| | | | 45° C.; 1xSSC, 50% | 0.3xSSC |
| | | | formamide | |
| D | DNA:RNA | <50 | T_{D}; 1xSSC | T_{D}; 1xSSC |
| E | RNA:RNA | >50 | 70° C.; 1xSSC -or- | 70° C.; |
| | | | 50° C.; 1xSSC, 50% | 0.3xSSC |
| | | | formamide | |
| F | RNA:RNA | <50 | T_{F}; 1xSSC | T_{f}; 1xSSC |
| G | DNA:DNA | >50 | 65° C.; 4xSSC -or- | 65° C.; 1xSSC |
| | | | 42° C.; 4xSSC, 50% | |
| | | | formamide | |
| H | DNA:DNA | <50 | T_{H}; 4xSSC | T_{H}; 4xSSC |
| I | DNA:RNA | >50 | 67° C.; 4xSSC -or- | 67° C.; 1xSSC |
| | | | 45° C.; 4xSSC, 50% | |
| | | | formamide | |
| J | DNA:RNA | <50 | T_{J}; 4xSSC | T_{J}; 4xSSC |
| K | RNA:RNA | >50 | 70° C.; 4xSSC -or- | 67° C.; 1xSSC |
| | | | 50° C.; 4xSSC, 50% | |
| | | | formamide | |
| L | RNA:RNA | <50 | T_{L}; 2xSSC | T_{L}; 2xSSC |
| M | DNA:DNA | >50 | 50° C.; 4xSSC -or- | 50° C.; 2xSSC |
| | | | 40° C.; 6xSSC, 50% | |
| | | | formamide | |
| N | DNA:DNA | <50 | T_{N}; 6xSSC | T_{N}; 6xSSC |
| O | DNA:RNA | >50 | 55° C.; 4xSSC -or- | 55° C.; 2xSSC |
| | | | 42° C.; 6xSSC, 50% | |
| | | | formamide | |
| P | DNA:RNA | <50 | T_{P}; 6xSSC | T_{P}; 6xSSC |
| Q | RNA:RNA | >50 | 60° C.; 4xSSC -or- | 60° C.; 2xSSC |
| | | | 45° C.; 6xSSC, 50% | |
| | | | formamide | |
| R | RNA:RNA | <50 | T_{R}; 4xSSC | T_{R}; 4xSSC |
| (bp)^{I}: The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length is determined by aligning the sequences of the polynucleotides and identifying the region or regions of optimal sequence complementarity. | | | | |
| Buffer^{H}: SSPE (1xSSPE is 0.15 M NaCl, 10 mM NaH₂PO₄, and 1.25 mM EDTA, pH 7.4) can be substituted for SSC (1xSSC is 0.15 M NaCl and 15 mM sodium citrate) in the hybridization and wash buffers; washes are performed for 15 minutes after hybridization is complete. | | | | |
| T_{B} through T_{R}: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 | | | | |
| #and 49 base pairs in length, Tₘ(° C.) = 81.5 + 16.6(log₁₀[Na⁺]) + 0.41(% G + C) − (600/N), where N is the number of bases in the hybrid, and [Na⁺] is the concentration of sodium ions in the hybridization buffer ([Na⁺] for 1xSSC = 0.165 M). | | | | |

B. Polypeptides

In certain embodiments, the invention is directed to methods for screening (or diagnosing) mood disorders in human subjects. In one particular embodiment, the invention is directed to a method of screening for a mood disorder in a human subject comprising (1) obtaining a biological sample from the subject; (2) contacting the sample with a plurality of antibodies, wherein the plurality of antibodies specifically bind an IGF-1 protein, a GMF-β protein, a CRMP2 protein, a PCTAIRE-3 protein, a HCNP protein, a hydroxysteroid sulfotransferase protein, a pyruvate dehydrogenase protein, an antioxidant protein-2 protein and a DDAH-1 protein; (3) measuring the amount of each antibody bound to its respective protein and (4) comparing the amount in step (3) with IGF-1, GMF-β, CRMP2, PCTAIRE-3, HCNP, hydroxysteroid sulfotransferase, pyruvate dehydrogenase, antioxidant protein-2 and DDAH-1 protein levels in human samples obtained from a statistically significant population lacking the mood disorder, wherein lower levels of one or more proteins in the subject indicates a predisposition to the mood disorder.

In certain other embodiments, the invention is directed to a method of screening for a mood disorder in a human subject comprising (1) obtaining a biological sample from a human subject; (2) applying the sample to an array of protein-capture agents, wherein at least one protein-capture agent on the array can bind a protein selected from the group consisting of human IGF-1, a human GMF-β, a human CRMP2, a human PCTAIRE-3, a human HCNP, a human hydroxysteroid sulfotransferase, a human pyruvate dehydrogenase-E1, a human antioxidant protein-2 and a human DDAH-1; (3) measuring the amount of each protein bound to its respective protein-capture agent and (4) comparing the level of the “captured” protein versus an array standard or control obtained from a statistically significant human population lacking the mood disorder.

Thus, in particular embodiments, the present invention provides isolated and purified IGF-1, GMF-β, CRMP2, PCTAIRE-3, HCNP, hydroxysteroid sulfotransferase, pyruvate dehydrogenase, antioxidant protein-2 and DDAH-1 polypeptides, or fragments thereof. Such isolated and purified polypeptides (or fragments thereof) are particularly useful as antigens in the generation of polyclonal or monoclonal antibodies of the invention (e.g., see Section C). Preferably, a full length polypeptide of the invention is a recombinant polypeptide. Typically, an IGF-1, a GMF-β, a CRMP2, a PCTAIRE-3, a HCNP, a hydroxysteroid sulfotransferase, a pyruvate dehydrogenase, an antioxidant protein-2 or a DDAH-1 polypeptide is produced by recombinant expression in a non-human cell. An IGF-1, a GMF-β, a CRMP2, a PCTAIRE-3, a HCNP, a hydroxysteroid sulfotransferase, a pyruvate dehydrogenase, an antioxidant protein-2 and/or a DDAH-1 polypeptide fragment of the invention may be recombinantly expressed or prepared via peptide synthesis methods known in the art (Barany et al., 1987; U.S. Pat. No. 5,258,454).

An IGF-1, a GMF-β, a CRMP2, a PCTAIRE-3, a HCNP, a hydroxysteroid sulfotransferase, a pyruvate dehydrogenase, an antioxidant protein-2 or a DDAH-1 polypeptide of the invention includes any functional variants of an IGF-1, a GMF-β, a CRMP2, a PCTAIRE-3, a HCNP, a hydroxysteroid sulfotransferase, a pyruvate dehydrogenase, an antioxidant protein-2 and/or a DDAH-1 polypeptide. Functional allelic variants are naturally occurring amino acid sequence variants of a human IGF-1, GMF-β, CRMP2, PCTAIRE-3, HCNP, hydroxysteroid sulfotransferase, pyruvate dehydrogenase, antioxidant protein-2 or a DDAH-1 polypeptide. Functional allelic variants typically contain only conservative substitution of one or more amino acids, or substitution, deletion or insertion of non-critical residues in non-critical regions of the polypeptide.

Modifications and changes are made in the structure of a polypeptide of the present invention and still obtain a molecule having IGF-1, GMF-β, CRMP2, PCTAIRE-3, HCNP, hydroxysteroid sulfotransferase, pyruvate dehydrogenase, antioxidant protein-2 or DDAH-1 polypeptide characteristics. For example, certain amino acids are substituted for other amino acids in a sequence without appreciable loss of receptor activity. Because it is the interactive capacity and nature of a polypeptide that defines that polypeptide's biological functional activity, certain amino acid sequence substitutions are made in a polypeptide sequence (or, of course, its underlying DNA coding sequence) and nevertheless obtain a polypeptide with like properties.

In making such changes, the hydropathic index of amino acids is considered. The importance of the hydropathic amino acid index in conferring interactive biologic function on a polypeptide is generally understood in the art (Kyte & Doolittle, 1982). It is known that certain amino acids can be substituted for other amino acids having a similar hydropathic index, or score, and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

As detailed in U.S. Pat. No. 4,554,101, the following hydrophilicity values have been assigned to amino acid residues: arginine (+3.0); lysine (+3.0); aspartate (+3.0±1); glutamate (+3.0+1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those which are within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions which take of the foregoing various characteristics into consideration are well known to those of skill in the art and include: arginine and lysine; glutamate and aspartate; serine and threonine; glutamine and asparagine; and valine, leucine and isoleucine (see Table 3 below). The present invention thus contemplates functional or biological equivalents of a polypeptide as set forth above.

TABLE 3
EXEMPLARY AMINO ACID SUBSTITUTIONS

| Original | Exemplary Residue |
|---|---|
| Residue | Substitution |
| Ala | Gly; Ser |
| Arg | Lys |
| Asn | Gln; His |
| Asp | Glu |
| Cys | Ser |
| Gln | Asn |
| Glu | Asp |
| Gly | Ala |
| His | Asn; Gln |
| Ile | Leu; Val |
| Leu | Ile; Val |
| Lys | Arg |
| Met | Leu; Tyr |
| Ser | Thr |
| Thr | Ser |
| Trp | Tyr |
| Tyr | Trp; Phe |
| Val | Ile; Leu |

Biological or functional equivalents of a polypeptide also are prepared using site-specific mutagenesis. Site-specific mutagenesis is a technique useful in the preparation of second generation polypeptides, or biologically functional equivalent polypeptides or peptides, derived from the sequences thereof, through specific mutagenesis of the underlying DNA. As noted above, such changes are desirable where amino acid substitutions are desirable. The technique further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

It is contemplated in the present invention, that a polypeptide of the invention is advantageously cleaved into fragments for use in the generation of reagents such as IGF-1, GMF-β, CRMP2, PCTAIRE-3, HCNP, hydroxysteroid sulfotransferase, pyruvate dehydrogenase, antioxidant protein-2 or DDAH-1 specific antibodies or for further structural or functional analysis. This is accomplished by treating purified or unpurified polypeptide with a protease such as glu-C (Boehringer, Indianapolis, Ind.), trypsin, chymotrypsin, V8 protease, pepsin and the like. Treatment with CNBr is another method by which fragments may be produced from natural IGF-1, GMF-β, CRMP2, PCTAIRE-3, HCNP, hydroxysteroid sulfotransferase, pyruvate dehydrogenase, antioxidant protein-2 or DDAH-1 polypeptides. Recombinant techniques also are used to express specific fragments of a polypeptide.

In addition, the invention contemplates that compounds sterically similar to IGF-1, GMF-β, CRMP2, PCTAIRE-3, HCNP, hydroxysteroid sulfotransferase, pyruvate dehydrogenase, antioxidant protein-2 or DDAH-1 are formulated to mimic the key portions of the peptide structure, called peptidomimetics or peptide mimetics. Mimetics are peptide-containing molecules which mimic elements of polypeptide secondary structure (e.g., see, U.S. Pat. No. 5,817,879, specifically incorporated herein by reference in its entirety). The underlying rationale behind the use of peptide mimetics is that the peptide backbone of polypeptides exists chiefly to orient amino acid side chains in such a way as to facilitate molecular interactions, such as those of receptor and ligand.

Successful applications of the peptide mimetics have thus far focused on mimetics of β-turns within polypeptides. β-turn structures within an IGF-1, GMF-β, CRMP2, PCTAIRE-3, HCNP, hydroxysteroid sulfotransferase, pyruvate dehydrogenase, antioxidant protein-2 or DDAH-1 polypeptide are predicted by computer-based algorithms. U.S. Pat. No. 5,933,819, specifically incorporated herein by reference in its entirety, describes a neural network based method and system for identifying relative peptide binding motifs from limited experimental data. In particular, an artificial neural network (ANN) is trained with peptides with known sequences and function (i.e., binding strength) identified from a phage display library. The ANN is then challenged with unknown peptides and predicts relative binding motifs. Analysis of the unknown peptides validate the predictive capability of the ANN. Once the component amino acids of the turn are determined, mimetics are constructed to achieve a similar spatial orientation of the essential elements of the amino acid side chains, as discussed in Johnson et al. (1993); U.S. Pat. No. 6,420,119 and U.S. Pat. No. 5,817,879, each incorporated herein by reference in its entirety.

C. Antibodies

In certain embodiments, the invention is directed to methods of screening for the up-regulation or down-regulation of one or more polypeptides selected from the group consisting of IGF-1, GMF-β, CRMP2, PCTAIRE-3, HCNP, hydroxysteroid sulfotransferase, pyruvate dehydrogenase, antioxidant protein-2 and DDAH-1. Thus, in particular embodiments, the invention is directed to a method of screening for a mood disorder in a human subject comprising (1) obtaining a biological sample from the subject; (2) contacting the sample with a plurality antibodies, wherein the plurality of antibodies specifically bind an IGF-1 protein, a GMF-β protein, a CRMP2 protein, a PCTAIRE-3 protein, a HCNP protein, a hydroxysteroid sulfotransferase protein, a pyruvate dehydrogenase protein, an antioxidant protein-2 protein and a DDAH-1 protein; (4) measuring the amount of each antibody bound to its respective protein and (4) comparing the amount in step (3) with IGF-1, GMF-β, CRMP2, PCTAIRE-3, HCNP, hydroxysteroid sulfotransferase, pyruvate dehydrogenase, antioxidant protein-2 and DDAH-1 protein levels in human samples obtained from a statistically significant population lacking the mood disorder, wherein lower levels of one or more proteins in the subject indicates a predisposition to the mood disorder. Thus, it is contemplated in this and other embodiments (e.g., as “protein-capture” agents, see Section F), that antibodies directed to one or more polypeptides selected from the group consisting of IGF-1, GMF-β, CRMP2, PCTAIRE-3, HCNP, hydroxysteroid sulfotransferase, pyruvate dehydrogenase, antioxidant protein-2 and DDAH-1 are particularly useful in such screening methods.

The present invention therefore provides antibodies immunoreactive with IGF-1, GMF-β, CRMP2, PCTAIRE-3, HCNP, hydroxysteroid sulfotransferase, pyruvate dehydrogenase, antioxidant protein-2 and DDAH-1 polypeptides. Preferably, the antibodies of the invention are monoclonal antibodies. Means for preparing and characterizing antibodies are well known in the art.

Briefly, a polyclonal antibody is prepared by immunizing an animal with an immunogen comprising a polypeptide of the present invention (i.e., a polypeptide comprising an amino acid sequence of SEQ ID NO:2, SEQ ID NO:4, SEQ ID NO:6, SEQ ID NO:8. SEQ ID NO:10, SEQ ID NO:12, SEQ ID NO:14, SEQ ID NO:16 or SEQ ID NO:18) and collecting antisera from that immunized animal. A wide range of animal species are used for the production of antisera. Typically an animal used for production of anti-antisera is a rabbit, a mouse, a rat, a hamster or a guinea pig. Because of the relatively large blood volume of rabbits, a rabbit is a preferred choice for production of polyclonal antibodies.

An antigen is typically defined on the basis of immunogenicity. Immunogenicity is defined as the ability to induce a humoral and/or cell-mediated immune response. Thus, the terms antigen or immunogen, as defined hereinafter, are molecules possessing the ability to induce a humoral and/or cell-mediated immune response.

As is well known in the art, a given polypeptide or polynucleotide varies in its immunogenicity. It is often necessary therefore to couple the immunogen (e.g., a polypeptide) of the invention with a carrier. Exemplary and preferred carriers are keyhole limpet hemocyanin (KLH), cholera holotoxin (CT), CRM₁₉₇, a mutant CT, E. coli heat labile toxin (LT), a mutant LT and bovine serum albumin (BSA). Other albumins such as ovalbumin, mouse serum albumin or rabbit serum albumin are also used as carriers.

Where a carrier protein and one or more antigens of the invention are conjugated (i.e., covalently associated), conjugation may be any chemical method, process or genetic technique commonly used in the art. For example, a CT carrier polypeptide and one or more antigens selected from IGF-1, GMF-β, CRMP2, PCTAIRE-3, HCNP, hydroxysteroid sulfotransferase, pyruvate dehydrogenase, antioxidant protein-2 and DDAH-1, may be conjugated by techniques, including, but not limited to: (1) direct coupling via protein functional groups (e.g., thiol-thiol linkage, amine-carboxyl linkage, amine-aldehyde linkage; enzyme direct coupling); (2) homobifunctional coupling of amines (e.g., using bis-aldehydes); (3) homobifunctional coupling of thiols (e.g., using bis-maleimides); (4) homobifunctional coupling via photoactivated reagents (5) heterobifunctional coupling of amines to thiols (e.g., using maleimides); (6) heterobifunctional coupling via photoactivated reagents (e.g., the β-carbonyldiazo family); (7) introducing amine-reactive groups into a poly- or oligosaccharide via cyanogen bromide activation or carboxymethylation; (8) introducing thiol-reactive groups into a poly- or oligosaccharide via a heterobifunctional compound such as maleimido-hydrazide; (9) protein-lipid conjugation via introducing a hydrophobic group into the protein and (10) protein-lipid conjugation via incorporating a reactive group into the lipid. Also, contemplated are heterobifunctional “non-covalent coupling” techniques such as the Biotin-Avidin interaction. For a comprehensive review of conjugation techniques, see Aslam and Dent (1998), incorporated hereinafter by reference in its entirety.

As is also well known in the art, immunogencity to a particular immunogen is enhanced by the use of non-specific stimulators of the immune response known as adjuvants. Exemplary and preferred adjuvants include complete Freund's adjuvant, incomplete Freund's adjuvant and aluminum hydroxide adjuvant.

The amount of immunogen used for the production of polyclonal antibodies varies inter alia, upon the nature of the immunogen as well as the animal used for immunization. A variety of routes are used to administer the immunogen (subcutaneous, intramuscular, intradermal, intravenous and intraperitoneal). The production of polyclonal antibodies is monitored by sampling blood of the immunized animal at various points following immunization. When a desired level of immunogenicity is obtained, the immunized animal is bled and the serum isolated and stored.

A monoclonal antibody of the present invention is readily prepared through use of well-known techniques such as those exemplified in U.S. Pat. No. 4,196,265, herein incorporated by reference in its entirety. Typically, a technique involves first immunizing a suitable animal with a selected antigen (e.g., a polypeptide of the present invention) in a manner sufficient to provide an immune response. Rodents such as mice and rats are preferred animals. Spleen cells from the immunized animal are then fused with cells of an immortal myeloma cell. Where the immunized animal is a mouse, a preferred myeloma cell is a murine NS-1 myeloma cell.

The fused spleen/myeloma cells are cultured in a selective medium to select fused spleen/myeloma cells from the parental cells. Fused cells are separated from the mixture of non-fused parental cells, e.g., by the addition of agents that block the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides. Where azaserine is used, the media is supplemented with hypoxanthine.

This culturing provides a population of hybridomas from which specific hybridomas are selected. Typically, selection of hybridomas is performed by culturing the cells by single-clone dilution in microtiter plates, followed by testing the individual clonal supernatants for reactivity with an antigen-polypeptide. The selected clones are then propagated indefinitely to provide the monoclonal antibody.

By way of specific example, to produce an antibody of the present invention, mice are injected intraperitoneally with between about 1-200 μg of an antigen comprising a polypeptide of the present invention. B lymphocyte cells are stimulated to grow by injecting the antigen in association with an adjuvant such as complete Freund's adjuvant (a non-specific stimulator of the immune response containing killed Mycobacterium tuberculosis). At some time (e.g., at least two weeks) after the first injection, mice are boosted by injection with a second dose of the antigen mixed with incomplete Freund's adjuvant.

A few weeks after the second injection, mice are tail bled and the sera titered by immunoprecipitation against radiolabeled antigen. Preferably, the process of boosting and titering is repeated until a suitable titer is achieved. The spleen of the mouse with the highest titer is removed and the spleen lymphocytes are obtained by homogenizing the spleen with a syringe. Typically, a spleen from an immunized mouse contains approximately 5×10⁷ to 2×10⁸ lymphocytes.

Mutant lymphocyte cells known as myeloma cells are obtained from laboratory animals in which such cells have been induced to grow by a variety of well-known methods. Myeloma cells lack the salvage pathway of nucleotide biosynthesis. Because myeloma cells are tumor cells, they can be propagated indefinitely in tissue culture, and are thus denominated immortal. Numerous cultured cell lines of myeloma cells from mice and rats, such as murine NS-1 myeloma cells, have been established.

Myeloma cells are combined under conditions appropriate to foster fusion with the normal antibody-producing cells from the spleen of the mouse or rat injected with the antigen/polypeptide of the present invention. Fusion conditions include, for example, the presence of polyethylene glycol. The resulting fused cells are hybridoma cells. Like myeloma cells, hybridoma cells grow indefinitely in culture.

Hybridoma cells are separated from unfused myeloma cells by culturing in a selection medium such as HAT media (hypoxanthine, aminopterin, thymidine). Unfused myeloma cells lack the enzymes necessary to synthesize nucleotides from the salvage pathway because they are killed in the presence of aminopterin, methotrexate, or azaserine. Unfused lymphocytes also do not continue to grow in tissue culture. Thus, only cells that have successfully fused (hybridoma cells) can grow in the selection media.

Each of the surviving hybridoma cells produces a single antibody. These cells are then screened for the production of the specific antibody immunoreactive with an antigen/polypeptide of the present invention. Single cell hybridomas are isolated by limiting dilutions of the hybridomas. The hybridomas are serially diluted many times and, after the dilutions are allowed to grow, the supernatant is tested for the presence of the monoclonal antibody. The clones producing that antibody are then cultured in large amounts to produce an antibody of the present invention in convenient quantity.

By use of a monoclonal antibody of the present invention, specific polypeptides of the invention are recognized as antigens, and thus identified. Once identified, those polypeptides are isolated and purified by techniques such as antibody-affinity chromatography. In antibody-affinity chromatography, a monoclonal antibody is bound to a solid substrate and exposed to a solution containing the desired antigen. The antigen is removed from the solution through an immunospecific reaction with the bound antibody. The polypeptide is then easily removed from the substrate and purified.

Additionally, examples of methods and reagents particularly amenable for use in generating and screening an antibody display library can be found in, for example, U.S. Pat. No. 5,223,409; International Application No. WO 92/18619; International Application No. WO 91/17271; International Application No. WO 92/20791; International Application No. WO 92/15679; International Application No. WO 93/01288; International Application No. WO 92/01047; International Application No. WO 92/09690; International Application No. WO 90/02809.

Additionally, recombinant antibodies, such as chimeric and humanized monoclonal antibodies, comprising both human and non-human fragments, which are made using standard recombinant DNA techniques, are within the scope of the invention. Such chimeric and humanized monoclonal antibodies are produced by recombinant DNA techniques known in the art, for example, using methods described in U.S. Pat. No. 6,054,297; European Application Nos. EP 184,187; EP 171,496; EP 173,494; International Application No. WO 86/01533; U.S. Pat. No. 4,816,567; and European Application No. EP 125,023.

An antibody (e.g., monoclonal antibody) is used to isolate the polypeptides by standard techniques, such as affinity chromatography or immunoprecipitation. An anti-IGF-1 antibody for example, can facilitate the purification of recombinantly produced IGF-1 polypeptide expressed in host cells. Moreover, an anti-IGF-1 antibody is used to detect IGF-1 polypeptide (e.g., in a biological sample, a cellular lysate or a cell supernatant) in order to evaluate the abundance of the polypeptide or the pattern of expression of the polypeptide.

Thus, anti-IGF-1, anti-GMF-β, anti-CRMP2, anti-PCTAIRE-3, anti-HCNP, anti-hydroxysteroid sulfotransferase, anti-pyruvate dehydrogenase, anti-antioxidant protein-2 and anti-DDAH-1 antibodies can be used diagnostically to monitor protein levels. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, P-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and acquorin, and examples of suitable radioactive material include ¹²⁵I, ¹³¹I, ¹⁵S or ³H.

D. Vectors, Host Cells and Recombinant Polypeptides

In certain embodiments, the present invention provides expression vectors expressing a polynucleotide complementary to an IGF-1 mRNA, a GMF-β mRNA, a CRMP2 mRNA, a PCTAIRE-3 mRNA, a HCNP mRNA, a hydroxysteroid sulfotransferase mRNA, a pyruvate dehydrogenase mRNA, an antioxidant protein-2 mRNA or a DDAH-1 mRNA. In certain other embodiments, the present invention provides expression vectors comprising a polynucleotide that encodes an IGF-1 polypeptide, a GMF-β polypeptide, a CRMP2 polypeptide, a PCTAIRE-3 polypeptide, a HCNP polypeptide, a hydroxysteroid sulfotransferase polypeptide, a pyruvate dehydrogenase polypeptide, an antioxidant protein-2 polypeptide or a DDAH-1 polypeptide. In still other embodiments, the invention provides a method for the treatment of a mood disorder which comprises delivering via a recombinant expression vector, one or more polynucleotides encoding one or more wild-type polypeptides selected from the group consisting of human IGF-1, a human GMF-β, a human CRMP2, a human PCTAIRE-3, a human HCNP, a human hydroxysteroid sulfotransferase, a human pyruvate dehydrogenase-E1, a human antioxidant protein-2 and a human DDAH-1.

Preferably, the expression vectors of the invention comprise polynucleotides operatively linked to an enhancer-promoter. In certain embodiments, the expression vectors of the invention comprise polynucleotides operatively linked to a prokaryotic promoter. Alternatively, the expression vectors of the present invention comprise polynucleotides operatively linked to an enhancer-promoter that is a eukaryotic promoter, and the expression vectors further comprise a polyadenylation signal that is positioned 3′ of the carboxy-terminal amino acid and within a transcriptional unit of the encoded polypeptide.

Expression of proteins in prokaryotes is most often carried out in E. coli with vectors containing constitutive or inducible promoters directing the expression of either fusion or non-fusion proteins. Fusion vectors add a number of amino acids to a protein encoded therein, to the amino or carboxy terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase.

Typical fusion expression vectors include pGEX (Pharmacia Biotech Inc; Smith and Johnson, 1988), pMAL (New England Biolabs, Beverly; MA) and pRIT5 (Pharmacia, Piscataway, N.J.) which fuse glutathione S-transferase (GST), maltose E binding protein, or protein A, respectively, to the target recombinant protein.

It is contemplated in certain embodiments, that a fusion expression vector or construct of the invention comprises a polynucleotide encoding an enhanced green fluorescent protein (EGFP) fused to a recombinant protein or polypeptide of the invention, wherein the EGFP facilitates the visualization of the recombinant protein or polypeptide.

Examples of suitable inducible non-fusion E. coli expression vectors include pTrc and pET-1 d (Studier et al., 1990). Target gene expression from the pTrc vector relies on host RNA polymerase transcription from a hybrid trp-lac fusion promoter. Target gene expression from the pET-11d vector relies on transcription from a T7 φ β-lac fusion promoter mediated by a coexpressed viral RNA polymerase T7. This viral polymerase is supplied by host strains BL21 (DE3) or HMS 174(DE3) from a resident prophage harboring a T7 RNA polymerase gene under the transcriptional control of the lacUV 5 promoter.

In another embodiment, the polynucleotide expression vector is a yeast expression vector. Examples of vectors for expression in yeast S. cerivisae include pYepSec I (Baldari, et al., 1987), pMFa (Kurjan and Herskowitz, 1982), pJRY88 (Schultz et al., 1987), and pYES2 (Invitrogen Corporation, San Diego, Calif.), p416GPD and p426GPD.

In yet another embodiment, a polynucleotide of the invention is expressed in mammalian cells using a mammalian expression vector. Examples of mammalian expression vectors include pCDM8 (Seed, 1987) and pMT2PC (Kaufman et al., 1987). When used in mammalian cells, the expression vector's control functions are often provided by viral regulatory elements.

For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., “Molecular Cloning: A Laboratory Manual” 2nd, ed, Cold Spring Harbor Laboratory, Cold Spring Harbor Laboratory Press, Cold Spring Harbor, N.Y., 1989, incorporated herein by reference.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non limiting examples of suitable tissue-specific promoters include the albumin promoter (liver-specific; Pinkert et al., 1987), lymphoid-specific promoters (Calame and Eaton, 1988), in particular promoters of T cell receptors (Winoto and Baltimore, 1989) and immunoglobulins (Banerji et al., 1983; Queen and Baltimore, 1983), neuron-specific promoters (e.g., the neurofilament promoter; Byrne and Ruddle, 1989), pancreas-specific promoters (Edlund et al., 1985), and mammary gland-specific promoters (e.g., milk whey promoter; U.S. Pat. No. 4,873,316 and European Application No. EP 264,166). Developmentally-regulated promoters are also encompassed, for example the murine hox promoters (Kessel and Gruss, 1990) and the α-fetoprotein promoter (Campes and Tilghman, 1989).

The regulatory sequence of the vector construct can be a constitutive promoter, an inducible promoter or a tissue specific promoter or an enhancer. The use of an inducible promoter will permit low basal levels of protein to be produced by the cell during routine culturing and expansion. Subsequently, the cells may then be induced to express large amounts of the desired protein during production or screening. The regulatory sequence may be isolated from cellular or viral genomes. Examples of cellular regulatory sequences include, but are not limited to, the actin gene, metallothionein I gene, collagen gene, serum albumin gene and immunoglobulin genes. Examples of viral regulatory sequences include, but are not limited to, regulatory elements from Cytomegalovirus (CMV) immediate early gene, adenovirus late genes, SV40 genes, retroviral LTRs and Herpesvirus genes (see Tables 3 and 4 for additional tissue specific and inducible regulatory sequences, respectively).

TABLE 3
TISSUE SPECIFIC PROMOTERS

| PROMOTER | Target |
|---|---|
| Tyrosinase | Melanocytes |
| Tyrosinase Related Protein, | Melanocytes |
| TRP-1 | |
| Prostate Specific Antigen, | Prostate Cancer |
| PSA | |
| Albumin | Liver |
| Apolipoprotein | Liver |
| Plasminogen Activator | Liver |
| Inhibitor Type-1, PAI-1 | |
| Fatty Acid Binding | Colon Epithelial Cells |
| Insulin | Pancreatic Cells |
| Muscle Creatine Kinase, | Muscle Cell |
| MCK | |
| Myelin Basic Protein, MBP | Oligodendrocytes and |
| | Glial Cells |
| Glial Fibrillary Acidic | Glial Cells |
| Protein, GFAP | |
| Neural Specific Enolase | Nerve Cells |
| Immunoglobulin Heavy | B-cells |
| Chain | |
| Immunoglobulin Light Chain | B-cells, |
| | Activated T-cells |
| T-Cell Receptor | Lymphocytes |
| HLA DQα and DQβ | Lymphocytes |
| β-Interferon | Leukocytes; |
| | Lymphocytes Fibroblasts |
| Interlukin-2 | Activated T-cells |
| Platelet Derived Growth | Erythrocytes |
| Factor | |
| E2F-1 | Proliferating Cells |
| Cyclin A | Proliferating Cells |
| α-, β-Actin | Muscle Cells |
| Haemoglobin | Erythroid Cells |
| Elastase I | Pancreatic Cells |
| Neural Cell Adhesion | Neural Cells |
| Molecule, NCAM | |

TABLE 4
INDUCIBLE PROMOTERS

| Promoter Element | Inducer |
|---|---|
| Early Growth Response-1 | Radiation |
| Gene, egr-1 | |
| Tissue Plasmingen | Radiation |
| Activator, t-PA | |
| fos and jun | Radiation |
| Multiple Drug Resistance | Chemotherapy |
| Gene 1, mdr-1 | |
| Heat Shock Proteins; | Heat |
| hsp16, hs60, hps68, | |
| hsp70, | |
| human Plasminogen | Tumor Necrosis Factor, |
| Activator Inhibitor type-1, | TNF |
| hPAI-1 | |
| Cytochrome P-450 | Toxins |
| CYP1A1 | |
| Metal-Responsive | Heavy Metals |
| Element, MRE | |
| Mouse Mammary Tumor | Glucocorticoids |
| Virus | |
| Collagenase | Phorbol Ester |
| Stromolysin | Phorbol Ester |
| SV40 | Phorbol Ester |
| Proliferin | Phorbol Ester |
| α-2-Macroglobulin | IL-6 |
| Murine MX Gene | Interferon, Newcastle |
| | Disease Virus |
| Vimectin | Serum |
| Thyroid Stimulating | Thyroid Hormone |
| Hormone α Gene | |
| HSP70 | Ela, SV40 Large T |
| | Antigen |
| Tumor Necrosis Factor | FMA |
| Interferon | Viral Infection, dsRNA |
| Somatostatin | Cyclic AMP |
| Fibronectin | Cyclic AMP |

A promoter is a region of a DNA molecule typically within about 100 nucleotide pairs in front of (upstream of) the point at which transcription begins (i.e., a transcription start site). That region typically contains several types of DNA sequence elements that are located in similar relative positions in different genes. As used herein, the term “promoter” includes what is referred to in the art as an upstream promoter region, a promoter region or a promoter of a generalized eukaryotic RNA Polymerase II transcription unit.

Another type of discrete transcription regulatory sequence element is an enhancer. An enhancer provides specificity of time, location and expression level for a particular encoding region (e.g., gene). A major function of an enhancer is to increase the level of transcription of a coding sequence in a cell that contains one or more transcription factors that bind to that enhancer. Unlike a promoter, an enhancer can function when located at variable distances from transcription start sites so long as a promoter is present.

As used herein, the phrase “enhancer-promoter” means a composite unit that contains both enhancer and promoter elements. An enhancer-promoter is operatively linked to a coding sequence that encodes at least one gene product. As used herein, the phrase “operatively linked” means that an enhancer-promoter is connected to a coding sequence in such a way that the transcription of that coding sequence is controlled and regulated by that enhancer-promoter. Means for operatively linking an enhancer-promoter to a coding sequence are well known in the art. As is also well known in the art, the precise orientation and location relative to a coding sequence whose transcription is controlled, is dependent inter alia upon the specific nature of the enhancer-promoter. Thus, a TATA box minimal promoter is typically located from about 25 to about 30 base pairs upstream of a transcription initiation site and an upstream promoter element is typically located from about 100 to about 200 base pairs upstream of a transcription initiation site. In contrast, an enhancer can be located downstream from the initiation site and can be at a considerable distance from that site.

A coding sequence of an expression vector is operatively linked to a transcription terminating region. RNA polymerase transcribes an encoding DNA sequence through a site where polyadenylation occurs. Typically, DNA sequences located a few hundred base pairs downstream of the polyadenylation site serve to terminate transcription. Those DNA sequences are referred to herein as transcription-termination regions. Those regions are required for efficient polyadenylation of transcribed messenger RNA (mRNA). Transcription-terminating regions are well known in the art. A preferred transcription-terminating region used in an adenovirus vector construct of the present invention comprises a polyadenylation signal of SV40 or the protamine gene.

The cell expressing or over-expressing the gene of interest can be cultured in vitro under conditions favoring the production of the desired amounts of the expression product. A cell containing a vector construct which has been integrated into its genome may also be introduced into a eukaryote (e.g., a vertebrate, preferably a mammal, more preferably a human) under conditions favoring the over-expression of the gene by the cell in vivo in the eukaryote.

Host cells can be derived from any eukaryotic species and can be primary, secondary, or immortalized. Furthermore, the cells can be derived from any tissue in the organism. Examples of useful tissues from which cells can be isolated and activated include, but are not limited to, liver, spleen, kidney, bone marrow, thymus, heart, muscle, lung, brain, testes, ovary, islet, intestinal, skin, gall bladder, prostate, bladder and the immune hemapoietic systems.

The vector construct can be integrated into primary, secondary, or immortalized cells. Primary cells are cells that have been isolated from a mammal and have not been passaged. Secondary cells are primary cells that have been passaged, but are not immortalized. Immortalized cells are cell lines that can be passaged, apparently indefinitely. Examples of immortalized cell lines include, but are not limited to, HT1080, HeLa, Jurkat, 293 cells, KB carcinoma, T84 colonic epithelial cell line, Raji, Hep G2 or Hep 3B, hepatoma cell lines, A2058 melanoma, U937 lymphoma and W138 fibroblast cell line, somatic cell hybrids and hybridomas.

Transfected cells of the present invention are useful in a number of applications in humans (e.g., ex vivo manipulation). In one embodiment, the cells are implanted into a human or an animal for polypeptide delivery (e.g., an IGF-1, GMF-β, CRMP2, PCTAIRE-3, HCNP, hydroxysteroid sulfotransferase, pyruvate dehydrogenase, antioxidant protein-2 or DDAH-1 polypeptide) in the human or animal. Methods for gene delivery and ex vivo cell manipulation are further described in Section F.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. A host cell is any prokaryotic or eukaryotic cell. For example, the polypeptide is expressed in bacterial cells such as E coli, insect cells, yeast or mammalian cells (such as Chinese hamster ovary cells (CHO), COS cells, NIH3T3 cells, NOS cells or PERC-6 cells). Other suitable host cells are known to those skilled in the art.

Vector DNA is introduced into prokaryotic or eukaryotic cells via conventional transformation, infection or transfection techniques. As used herein, the terms “transformation”, “infection” and “transfection” are intended to refer to a variety of art-recognized techniques for introducing foreign nucleic acid (e.g., exogenous DNA) into a host cell, including calcium phosphate or calcium chloride transfection, DEAE-dextran-mediated transfection, lipofection, protoplast fusion, liposome-mediated transfection, direct microinjection, adenovirus infection, or electroporation. Suitable methods for transforming, infecting or transfecting host cells can be found in Sambrook et al. (“Molecular Cloning: A Laboratory Manual” 2nd ed, Cold Spring Harbor Laboratory, Cold Spring Harbor Laboratory Press, Cold Spring Harbor, N.Y., 1989), and other laboratory manuals.

A host cell of the invention, such as a prokaryotic or eukaryotic host cell in culture, is used to produce (i.e., express) polypeptides of the invention. Accordingly, the invention further provides methods for producing polypeptides using the host cells of the invention. In one embodiment, the method comprises culturing the host cell of invention (into which a recombinant expression vector encoding a polypeptide has been introduced) in a suitable medium until the polypeptide is produced. In another embodiment, the method further comprises isolating the polypeptide from the medium or the host cell.

The most widely used method is transfection mediated by either calcium phosphate or DEAE-dextran. Although the mechanism remains obscure, it is believed that the transfected DNA enters the cytoplasm of the cell by endocytosis and is transported to the nucleus. Depending on the cell type, up to 90% of a population of cultured cells can be transfected at any one time. Because of its high efficiency, transfection mediated by calcium phosphate or DEAE-dextran is the method of choice for experiments that require transient expression of the foreign DNA in large numbers of cells. Calcium phosphate-mediated transfection is also used to establish cell lines that integrate copies of the foreign DNA, which are usually arranged in head-to-tail tandem arrays into the host cell genome.

In the protoplast fusion method, protoplasts derived from bacteria carrying high numbers of copies of a plasmid of interest are mixed directly with cultured mammalian cells. After fusion of the cell membranes (usually with polyethylene glycol), the contents of the bacteria are delivered into the cytoplasm of the mammalian cells and the plasmid DNA is transported to the nucleus. Protoplast fusion is not as efficient as transfection for many of the cell lines that are commonly used for transient expression assays, but it is useful for cell lines in which endocytosis of DNA occurs inefficiently. Protoplast fusion frequently yields multiple copies of the plasmid DNA tandemly integrated into the host chromosome.

The application of brief, high-voltage electric pulses to a variety of mammalian and plant cells leads to the formation of nanometer-sized pores in the plasma membrane. DNA is taken directly into the cell cytoplasm either through these pores or as a consequence of the redistribution of membrane components that accompanies closure of the pores. Electroporation can be extremely efficient and can be used both for transient expression of cloned genes and for establishment of cell lines that carry integrated copies of the gene of interest. Electroporation, in contrast to calcium phosphate-mediated transfection and protoplast fusion, frequently gives rise to cell lines that carry one, or at most a few, integrated copies of the foreign DNA.

Liposome transfection involves encapsulation of DNA and RNA within liposomes, followed by fusion of the liposomes with the cell membrane. The mechanism of how DNA is delivered into the cell is unclear but transfection efficiencies can be as high as 90%.

Direct microinjection of a DNA molecule into nuclei has the advantage of not exposing DNA to cellular compartments such as low-pH endosomes. Microinjection is therefore used primarily as a method to establish lines of cells that carry integrated copies of the DNA of interest.

The use of adenovirus as a vector for cell transfection is well known in the art (e.g., see Section F and U.S. Pat. No. 5,928,944) and has been reported for various cells.

E. Transgenic Animals

In certain embodiments, the invention pertains to transgenic, non-human animals comprising one or more exogenous polynucleotides encoding a protein selected from the group consisting of an IGF-1 protein, a GMF-β protein, a CRMP2 protein, a PCTAIRE-3 protein, a HCNP protein, a hydroxysteroid sulfotransferase protein, a pyruvate dehydrogenase protein, an antioxidant protein-2 or a DDAH-1 protein. In other embodiments, the invention is directed to transgenic, non-human animals having a functional disruption in one or more genes encoding a protein selected from the group consisting of an IGF-1 protein, a GMF-β protein, a CRMP2 protein, a PCTAIRE-3 protein, a HCNP protein, a hydroxysteroid sulfotransferase protein, a pyruvate dehydrogenase protein, an antioxidant protein-2 or a DDAH-1 protein. In other embodiments, the invention is directed to screening for a monoamine re-uptake inhibitor or activator in one of these transgenic non-human animals.

Thus, the transgenic animals of the invention are useful, for example, as standard controls by which to evaluate monoamine re-uptake inhibitors, as recipients of a normal human IGF-1, GMF-β, CRMP2, PCTAIRE-3, HCNP, hydroxysteroid sulfotransferase, pyruvate dehydrogenase, antioxidant protein-2 or DDAH-1 gene to thereby create a model system for screening monoamine re-uptake inhibitors in vivo, and to identify mood disorders for treatment with monoamine re-uptake inhibitors. The animals are also useful as controls for studying the effect of monoamine re-uptake inhibitors such as fluoxetine and venlafaxine on the expression patterns or levels of human IGF-1, GMF-β, CRMP2, PCTAIRE-3, HCNP, hydroxysteroid sulfotransferase, pyruvate dehydrogenase, antioxidant protein-2 and/or DDAH-1 genes and polypeptides.

As used herein, a “transgene” is an exogenous polynucleotide which is integrated into the genome of a cell from which a transgenic animal develops and which remains in the genome of the mature animal, thereby directing the expression of an encoded gene product in one or more cell types or tissues of the transgenic animal. Thus, it is contemplated that in some instances the genome of a transgenic animal of the invention is altered through the stable introduction of one or more of the polynucleotide compositions described herein, either native, synthetically modified or mutated.

As defined herein, a “transgenic animal” is a non-human animal (e.g. mouse, rat, rabbit, squirrel, hamster, rabbits, guinea pigs, pigs, micro-pigs, prairie dog, baboons, squirrel monkeys and chimpanzees, etc), preferably a mammal, more preferably a rodent such as a rat or mouse, in which one or more of the cells of the animal include a transgene introduced by way of human intervention, such as by transgenic techniques well known in the art. The transgene is introduced into the cell, directly or indirectly, by introduction into a precursor cell, by way of deliberate genetic manipulation, such as microinjection or infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA.

In one embodiment, the gene of a transgenic non-human animal is disrupted by homologous recombination between the endogenous allele and an exogenous mutant polynucleotide, or portion thereof, that has been introduced into an embryonic stem cell precursor of the animal. The embryonic stem cell precursor is then allowed to develop, resulting in an animal having a functionally disrupted gene. The animal may have one gene allele functionally disrupted (i.e., the animal may be heterozygous for the mutation), or more preferably, the animal has both gene alleles functionally disrupted (i.e., the animal can be homozygous for the mutation).

In one embodiment of the invention, a functional disruption of both gene alleles produces animals in which expression of the gene product in cells of the animal is substantially absent relative to non-mutant (i.e., wild-type) animals. In another embodiment, the gene alleles are disrupted such that an altered (i.e., mutated) gene product is produced in cells of the animal. A preferred non-human animal of the invention having a functionally disrupted gene is a mouse. Given the essentially complete inactivation of protein function in the homozygous animals of the invention and the about 50% inhibition of protein function in the heterozygous animals of the invention, these animals are useful as positive controls against which to evaluate the effectiveness of monoamine re-uptake inhibitors.

In another embodiment, the invention pertains to a transgenic nonhuman animal having (1) a functionally disrupted endogenous IGF-1, GMF-β, CRMP2, PCTAIRE-3, HCNP, hydroxysteroid sulfotransferase, pyruvate dehydrogenase, antioxidant protein-2 or DDAH-1 gene and (2) a transgene encoding a heterologous IGF-1, GMF-β, CRMP2, PCTAIRE-3, HCNP, hydroxysteroid sulfotransferase, pyruvate dehydrogenase, antioxidant protein-2 or DDAH-1 gene (i.e., a gene from another species). Preferably, the animal is a mouse and the heterologous gene is a human IGF-1, GMF-β, CRMP2, PCTAIRE-3, HCNP, hydroxysteroid sulfotransferase, pyruvate dehydrogenase, antioxidant protein-2 or DDAH-1 gene. An animal of the invention which has been reconstituted with human IGF-1, GMF-β, CRMP2, PCTAIRE-3, HCNP, hydroxysteroid sulfotransferase, pyruvate dehydrogenase, antioxidant protein-2 and/or DDAH-1 can be used to identify agents that inhibit or activate a monoamine re-uptake receptor. For example, a compound that induces monoamine receptor re-uptake activity is administered to the transgenic animal and a wild-type animal, and the animal response is measured or monitored.

Thus, in certain embodiments, the invention is directed to a polynucleotide construct for functionally disrupting an IGF-1, GMF-β, CRMP2, PCTAIRE-3, HCNP, hydroxysteroid sulfotransferase, pyruvate dehydrogenase, antioxidant protein-2 or DDAH-1 gene in a host cell. The nucleic acid construct comprises: a) a nonhomologous replacement portion; b) a first homology region located upstream of the nonhomologous replacement portion, the first homology region having a nucleotide sequence with substantial identity to a first IGF-1, GMF-β, CRMP2, PCTAIRE-3, HCNP, hydroxysteroid sulfotransferase, pyruvate dehydrogenase, antioxidant protein-2 or DDAH-1 gene sequence; and c) a second homology region located downstream of the nonhomologous replacement portion, the second homology region having a nucleotide sequence with substantial identity to a second IGF-1, GMF-β, CRMP2, PCTAIRE-3, HCNP, hydroxysteroid sulfotransferase, pyruvate dehydrogenase, antioxidant protein-2 or DDAH-1 gene sequence, the second IGF-1, GMF-β, CRMP2, PCTAIRE-3, HCNP, hydroxysteroid sulfotransferase, pyruvate dehydrogenase, antioxidant protein-2 or DDAH-1 gene sequence having a location downstream of the first IGF-1, GMF-β, CRMP2, PCTAIRE-3, HCNP, hydroxysteroid sulfotransferase, pyruvate dehydrogenase, antioxidant protein-2 or DDAH-1 gene sequence in a naturally occurring endogenous IGF-1, GMF-β, CRMP2, PCTAIRE-3, HCNP, hydroxysteroid sulfotransferase, pyruvate dehydrogenase, antioxidant protein-2 or DDAH-1 gene.

Additionally, the first and second homology regions are of sufficient length for homologous recombination between the nucleic acid construct and an endogenous IGF-1, GMF-β, CRMP2, PCTAIRE-3, HCNP, hydroxysteroid sulfotransferase, pyruvate dehydrogenase, antioxidant protein-2 or DDAH-1 gene in a host cell when the nucleic acid molecule is introduced into the host cell. As used herein, a “homologous recombinant animal” is a non-human animal, preferably a mammal, more preferably a mouse, in which an endogenous gene has been altered by homologous recombination between the endogenous gene and an exogenous polynucleotide molecule introduced into a cell of the animal, e.g., an embryonic cell of the animal, prior to development of the animal.

In a preferred embodiment, the nonhomologous replacement portion comprises a positive selection expression cassette, preferably including a neomycin phosphotransferase gene operatively linked to a regulatory element(s). In another preferred embodiment, the nucleic acid construct also includes a negative selection expression cassette distal to either the upstream or downstream homology regions. A preferred negative selection cassette includes a herpes simplex virus thymidine kinase gene operatively linked to a regulatory element(s). Another aspect of the invention pertains to recombinant vectors into which the nucleic acid construct of the invention has been incorporated.

Yet another aspect of the invention pertains to host cells into which the nucleic acid construct of the invention is introduced to thereby allow homologous recombination between the nucleic acid construct and an endogenous gene of the host cell, resulting in functional disruption of the endogenous gene. The host cell is a mammalian cell that normally expresses IGF-1, GMF-β, CRMP2, PCTAIRE-3, HCNP, hydroxysteroid sulfotransferase, pyruvate dehydrogenase, antioxidant protein-2 or DDAH-1, such as a human neuron, or a pluripotent cell, such as a mouse embryonic stem cell. Further development of an embryonic stem cell into which the nucleic acid construct is introduced and homologously recombined with the endogenous IGF-1, GMF-β, CRMP2, PCTAIRE-3, HCNP, hydroxysteroid sulfotransferase, pyruvate dehydrogenase, antioxidant protein-2 or DDAH-1 gene produces a transgenic nonhuman animal having cells that are descendant from the embryonic stem cell and thus carry the gene disruption in their genome. Animals that carry the IGF-1, GMF-β, CRMP2, PCTAIRE-3, HCNP, hydroxysteroid sulfotransferase, pyruvate dehydrogenase, antioxidant protein-2 or DDAH-1 gene disruption in their germline are then selected and bred to produce animals having the gene disruption in all somatic and germ cells. Such mice are then bred to homozygosity for the IGF-1, GMF-β, CRMP2, PCTAIRE-3, HCNP, hydroxysteroid sulfotransferase, pyruvate dehydrogenase, antioxidant protein-2 or DDAH-1 gene disruption.

A transgenic animal of the invention can be created by introducing an IGF-1, GMF-β, CRMP2, PCTAIRE-3, HCNP, hydroxysteroid sulfotransferase, pyruvate dehydrogenase, antioxidant protein-2 or DDAH-1 nucleic acid into the male pronuclei of a fertilized oocyte, e.g., by microinjection, retroviral infection, and allowing the oocyte to develop in a pseudopregnant female foster animal. The human IGF-1, GMF-β, CRMP2, PCTAIRE-3, HCNP, hydroxysteroid sulfotransferase, pyruvate dehydrogenase, antioxidant protein-2 or DDAH-1 cDNA sequence is introduced as a transgene into the genome of a non-human animal.

To create a homologous recombinant animal, a vector is prepared which contains at least a fragment of a IGF-1, GMF-β, CRMP2, PCTAIRE-3, HCNP, hydroxysteroid sulfotransferase, pyruvate dehydrogenase, antioxidant protein-2 or DDAH-1 gene into which a deletion, addition or substitution has been introduced to thereby alter, e.g., functionally disrupt the IGF-1, GMF-β, CRMP2, PCTAIRE-3, HCNP, hydroxysteroid sulfotransferase, pyruvate dehydrogenase, antioxidant protein-2 or DDAH-1 gene. The gene is a human gene (e.g., from a human genomic clone isolated from a human genomic library), but more preferably is a non-human homologue of a human IGF-1, GMF-β, CRMP2, PCTAIRE-3, HCNP, hydroxysteroid sulfotransferase, pyruvate dehydrogenase, antioxidant protein-2 or DDAH-1 gene. In a particular embodiment, the vector is designed such that, upon homologous recombination, the endogenous gene is functionally disrupted (i.e., no longer encodes a functional protein; also referred to as a “knock out” vector.

Alternatively, the vector is designed such that, upon homologous recombination, the endogenous gene is mutated or otherwise altered but still encodes functional protein (e.g., the upstream regulatory region can be altered to thereby alter the expression of the endogenous IGF-1, GMF-β, CRMP2, PCTAIRE-3, HCNP, hydroxysteroid sulfotransferase, pyruvate dehydrogenase, antioxidant protein-2 or DDAH-1 polypeptide). In the homologous recombination vector, the altered fragment of the IGF-1, GMF-β, CRMP2, PCTAIRE-3, HCNP, hydroxysteroid sulfotransferase, pyruvate dehydrogenase, antioxidant protein-2 or DDAH-1 gene is flanked at its 5′ and 3′ ends by additional nucleic acid of the IGF-1, GMF-β, CRMP2, PCTAIRE-3, HCNP, hydroxysteroid sulfotransferase, pyruvate dehydrogenase, antioxidant protein-2 or DDAH-1 to allow for homologous recombination to occur between the exogenous gene carried by the vector and a gene in an embryonic stem cell. The additional flanking IGF-1, GMF-β, CRMP2, PCTAIRE-3, HCNP, hydroxysteroid sulfotransferase, pyruvate dehydrogenase, antioxidant protein-2 or DDAH-1 nucleic acid is of sufficient length for successful homologous recombination with the endogenous gene.

Typically, several kilobases of flanking DNA (both at the 5′ and 3′ ends) are included in the vector (see e.g., Thomas and Capecchi, 1987, for a description of homologous recombination vectors). The vector is introduced into an embryonic stem cell line (e.g., by electroporation) and cells in which the introduced IGF-1, GMF-β, CRMP2, PCTAIRE-3, HCNP, hydroxysteroid sulfotransferase, pyruvate dehydrogenase, antioxidant protein-2 or DDAH-1 gene has homologously recombined with the endogenous gene are selected. The selected cells are then injected into a blastocyst of an animal (e.g., a mouse) to form aggregation chimeras (see e.g., Bradley, 1987). A chimeric embryo is then implanted into a suitable pseudopregnant female foster animal and the embryo brought to term. Progeny harboring the homologously recombined DNA in their germ cells are used to breed animals in which all cells of the animal contain the homologously recombined DNA by germline transmission of the transgene. Methods for constructing homologous recombination vectors and homologous recombinant animals are described further in Bradley, 1991; and in PCT International Application Nos. WO 90/11354; WO 91/01140; and WO 93/04169.

In another embodiment, transgenic non-human animals are produced which contain selected systems which allow for regulated expression of the transgene. One example of such a system is the cre/loxP recombinase system of bacteriophage PL. For a description of the cre/loxP recombinase system, see, e.g., Lakso et al., 1992. Another example of a recombinase system is the FLP recombinase system of Saccharomyces cerevisiae (O'Gonnan et al., 1991). If a cre/loxP recombinase system is used to regulate expression of the transgene, animals containing transgenes encoding both the Cre recombinase and a selected protein are required. Such animals are provided through the construction of “double” transgenic animals, e.g., by mating two transgenic animals, one containing a transgene encoding a selected protein and the other containing a transgene encoding a recombinase.

Clones of the non-human transgenic animals described herein are also produced according to the methods described in Wilmut et al., 1997, and PCT International Application Nos. WO 97/07668 and WO 97/07669. In brief, a cell, e.g., a somatic cell, from the transgenic animal is isolated and induced to exit the growth cycle and enter Gₒ phase. The quiescent cell is then fused, e.g., through the use of electrical pulses, to an enucleated oocyte from an animal of the same species from which the quiescent cell is isolated. The reconstructed oocyte is then cultured such that it develops to morula or blastocyst and then transferred to pseudopregnant female foster animal. The offspring borne of this female foster animal will be a clone of the animal from which the cell, e.g., the somatic cell, is isolated.

F. Methods and Uses of the Invention

In certain embodiments, the invention is directed to methods of screening a human subject for a mood disorder, wherein the subject has reduced expression levels of one or more mRNA selected from the group consisting of human IGF-1, a human GMF-β, a human CRMP2, a human PCTAIRE-3, a human HCNP, a human hydroxysteroid sulfotransferase, a human pyruvate dehydrogenase-E1, a human antioxidant protein-2 and a human DDAH-1, or alternatively, reduced expression levels of the polypeptide encoded by the mRNA.

Thus, in certain embodiments, the invention is directed to a method of screening for a mood disorder in a human subject via a polynucleotide probe complementary to an mRNA selected from the group consisting of an IGF-1 mRNA, a GMF-β mRNA, a CRMP2 mRNA, a PCTAIRE-3 mRNA, a HCNP mRNA, a hydroxysteroid sulfotransferase mRNA, a pyruvate dehydrogenase mRNA, an antioxidant protein-2 mRNA and a DDAH-1.

In certain other embodiments, the invention is directed to a method of screening for a mood disorder in a human subject via a plurality polynucleotide probes, wherein the probes are complementary to an IGF-1 mRNA, a GMF-β mRNA, a CRMP2 mRNA, a PCTAIRE-3 mRNA, a HCNP mRNA, a hydroxysteroid sulfotransferase mRNA, a pyruvate dehydrogenase mRNA, an antioxidant protein-2 mRNA and a DDAH-1 mRNA.

As defined hereinafter, a “body fluid” may be any liquid substance extracted, excreted, or secreted from a mammal, a tissue of a mammal or a cell of a mammal. The body fluid need not necessarily contain cells. Body fluids of relevance to the present invention include, but are not limited to, whole blood, blood plasma, serum, erythrocytes, leukocytes, platelets, lymphocytes, macrophages, fibroblast cells, mast cells, fat cells, epithelial cells, nerve cells, glial cells, Schwann cells, progenitor stem cells, urine, plasma, cerebrospinal fluid (CSF), tears, sinovial fluid, amniotic fluid and saliva. As defined hereinafter, a “biological sample” includes the body fluids set forth above, and further includes other mammalian tissues or tissue samples such as a skin biopsy, a brain biopsy or a buccal biopsy.

1. Polynucleotide Arrays

In certain embodiments, the invention is directed to methods of screening for a mood disorder in a human subject. Thus, in a particular embodiment, the invention is directed to a method of screening for a mood disorder in a human subject comprising (1) obtaining a biological sample from a human subject; (2) applying the sample to an array of oligonucleotide probes, wherein at least one or more probes on the array can bind a polynucleotide selected from the group consisting of human IGF-1, a human GMF-β, a human CRMP2, a human PCTAIRE-3, a human HCNP, a human hydroxysteroid sulfotransferase, a human pyruvate dehydrogenase-E1, a human antioxidant protein-2 and a human DDAH-1; (3) measuring the amount of each polynucleotide bound to its respective oligonucleotide probe; and (4) comparing the level of the bound probe(s) versus an array standard or a control obtained from a statistically significant population of human subjects lacking a mood disorder. In a preferred embodiment, the array of oligonucleotide probes comprises at least a probe which binds IGF-1, GMF-β, CRMP2, PCTAIRE-3, HCNP, hydroxysteroid sulfotransferase, pyruvate dehydrogenase-E1, antioxidant protein-2 and DDAH-1.

Thus, in certain embodiments the polynucleotides according to the invention are used in analytical “DNA” chips which allow sequencing, the study of mutations and the expression of genes. The principle of the operation of these chips is based on molecular probes, most often oligonucleotides, which are attached onto a miniaturized surface, generally of the order of a few square centimeters. During an analysis, a sample containing fragments of a target nucleic acid to be analyzed, for example, DNA or RNA labelled, after amplification, is deposited onto the DNA chip in which the support has been coated beforehand with probes. Bringing the labelled target sequences into contact with the probes leads to the formation, through hybridization, of a duplex according to the rule of Watson and Crick base pairing. After a washing step, analysis of the surface of the chip allows the effective hybridizations to be located by means of the signals emitted by the labels tagging the target. A hybridization fingerprint results from this analysis which, by appropriate computer processing, will make it possible to determine information such as the presence of specific fragments in the sample, the determination of sequences and the presence of mutations.

The chip consists of a multitude of molecular probes, precisely organized or arrayed on a solid support whose surface is miniaturized. It is at the centre of a system where other elements (imaging system, microcomputer) allow the acquisition and interpretation of a hybridization fingerprint.

The hybridization supports are provided in the form of flat or porous surfaces (pierced with wells) composed of various materials. The choice of a support is determined by its physicochemical properties, or more precisely, by the relationship between the latter and the conditions under which the support will be placed during the synthesis or the attachment of the probes or during the use of the chip. It is therefore necessary, before considering the use of a particular support, to consider characteristics such as its stability to pH, its physical strength, its reactivity and its chemical stability as well as its capacity to nonspecifically bind nucleic acids. Materials such as glass, silicon and polymers are commonly used. Their surface is, in a first step called “functionalization”, made reactive towards the groups which it is desired to attach thereon. After the functionalization, so-called spacer molecules are grafted onto the activated surface. Used as intermediates between the surface and the probe, these molecules of variable size render unimportant the surface properties of the supports, which often prove to be problematic for the synthesis or the attachment of the probes and for the hybridization.

Among the hybridization supports, glass often is used, for example, in the method of in situ synthesis of oligonucleotides by photochemical addressing developed by the company Affymetrix (Santa Clara, Calif.), the glass surface being activated by silane. Genosensor Consortium (The Woodlands, Tex.) also uses glass slides carrying wells 3 mm apart, this support being activated with epoxysilane.

The probes according to the invention may be synthesized directly in situ on the supports of the DNA chips. This in situ synthesis may be carried out by photochemical addressing (developed by the company Affymax (Amsterdam, Holland) and exploited industrially by its subsidiary Affymetrix (Santa Clara, Calif.)), or based on the VLSIPS (very large scale immobilized polymer synthesis) technology (Fodor et al., 1991), which is based on a method of photochemically directed combinatory synthesis, the principle of which combines solid-phase chemistry, the use of photolabile protecting groups and photolithography.

The probes according to the invention may be attached to the DNA chips in various ways such as electrochemical addressing, automated addressing or the use of probe printers (Livache et al., 1994; Yershov et al., 1996 and Derisi et al., 1996).

The revealing of the hybridization between the probes of the invention, deposited or synthesized in situ on the supports of the DNA chips, and the sample to be analyzed, may be determined, for example, by measurement of fluorescent signals, by radioactive counting or by electronic detection. The use of fluorescent molecules such as fluorescein (e.g., fluorescein isothiocyanate, FITC) constitutes the most common method of labelling the samples. It allows direct or indirect revealing of the hybridization and allows the use of various fluorochromes.

Affymetrix currently provides an apparatus or a scanner designed to read its Gene Chip® chips. It makes it possible to detect the hybridizations by scanning the surface of the chip in confocal microscopy (Lipshutz et al., 1995).

Thus, the nucleotide sequences according to the invention are used in DNA chips to carry out the analysis of one or more genes selected from the group consisting of human IGF-1, a human GMF-β, a human CRMP2, a human PCTAIRE-3, a human HCNP, a human hydroxysteroid sulfotransferase, a human pyruvate dehydrogenase-E1, a human antioxidant protein-2 and a human DDAH-1. Analysis of the expression of these genes is based on the use of chips where probes of the invention, chosen for their specificity to characterize a given gene, are present (Lockhart et al., 1996; Shoemaker et al., 1996). For the methods of analysis of gene expression using the DNA chips, reference may, for example, be made to the methods described by Lockhart et al. (1996) for the synthesis of probes in situ or for the addressing and the attachment of previously synthesized probes. The target sequences to be analyzed are labelled and in general fragmented into sequences of about 50 to 100 nucleotides before being hybridized onto the chip. After washing as described, for example, by Lockhart et al. (1996) and application of different electric fields, the labelled compounds are detected and quantified, the hybridizations being carried out at least in duplicate. Comparative analyses of the signal intensities obtained with respect to the same probe for different samples and/or for different probes with the same sample, determine the differential expression of RNA or copy numbers of DNA derived from the sample.

Accordingly, the subject of the invention is also the nucleotide sequences according to the invention, characterized in that they are immobilized on a support of a DNA chip. The DNA chips, characterized in that they contain at least one nucleotide sequence according to the invention immobilized on the support of the said chip, also form part of the invention.

The chips will preferably contain several probes or nucleotide sequences of the invention of different length and/or corresponding to different genes so as to identify, with greater certainty, the specificity of the target sequences or the desired mutation in the sample to be analyzed.

2. Protein-Capture Arrays

In certain embodiments, the invention is directed to methods of detecting or screening for a mood disorder in a human subject comprising: (1) obtaining a biological sample from a human subject; (2) applying the sample to an array of protein-capture agents, wherein at least one protein-capture agent on the array can bind a protein selected from the group consisting of human IGF-1, human GMF-β, human CRMP2, human PCTAIRE-3, human HCNP, human hydroxysteroid sulfotransferase, human pyruvate dehydrogenase-E1, human antioxidant protein-2 and human DDAH-1; (3) measuring the amount of each protein bound to its respective protein-capture agent and (4) comparing the level of the “captured” protein in step (3) versus an array standard or control obtained from a statistically significant population of human subjects lacking a mood disorder. In a preferred embodiment, the array of protein-capture agents comprises at least a protein-capture agent which binds human IGF-1, a protein-capture agent which binds human GMF-β, a protein-capture agent which binds human CRMP2, a protein-capture agent which binds human PCTAIRE-3, a protein-capture agent which binds human HCNP, a protein-capture agent which binds human hydroxysteroid sulfotransferase, a protein-capture agent which binds human pyruvate dehydrogenase-E1, a protein-capture agent which binds human antioxidant protein-2 and a protein-capture agent which binds human DDAH-1.

Thus, in certain embodiments, the invention is directed to an array of protein-capture agents which can bind a plurality of proteins that are the expression products (or fragments thereof) of a cell (or population of cells) in a human subject and therefore can be used to evaluate gene expression at the protein level.

As defined hereinafter, the term “protein-capture agent” means a molecule or a multi-molecular complex which can bind a protein to itself. Protein-capture agents preferably bind their binding partners in a substantially specific manner. In preferred embodiments, an array comprises at least a protein-capture agent specific for a human IGF-1, a human GMF-β, a human CRMP2, a human PCTAIRE-3, a human HCNP, a human hydroxysteroid sulfotransferase, a human pyruvate dehydrogenase-E1, a human antioxidant protein-2 and a human DDAH-1.

Protein-capture agents with a dissociation constant (K_{d}) of less than about 10⁻⁶ are preferred. The protein-capture agent will most typically be a biomolecule such as a protein. The biomolecule may optionally be a naturally occurring, recombinant, or synthetic biomolecule. Antibodies or antibody fragments are highly suitable as protein-capture agents. Antigens may also serve as protein-capture agents, since they are capable of binding antibodies. A receptor which binds a protein ligand is another example of a possible protein-capture agent. For instance, protein-capture agents are understood not to be limited to agents which only interact with their binding partners through noncovalent interactions. Protein-capture agents may also optionally become covalently attached to proteins which they bind. For instance, the protein-capture agent may be photocrosslinked to its binding partner following binding.

Protein-capture arrays are known in the art (e.g., see U.S. Pat. No. 6,365,418, U.S. Pat. No. 6,475,808 and U.S. Pat. No. 6,475,809, each specifically incorporated herein by reference in its entirety) and are available as “array binding formats” such as (a) direct binding arrays (Chiphergen Biosystems, Inc. (Freemont, Calif.)), (Biacore Inc. (Piscataway, N.J.)); (b) ligand arrays (SomaLogic, Inc., (Boulder, Colo.)); (c) antibody arrays (Pierce Biotechnology, Inc., (Rockford, Ill.)), (Zyomyx, Inc., (Hayward, Calif.)), (Cambridge Antibody Technology, (Cambridge, UK)), (Milagen, Inc., (Richmond, Calif.)); and (4) antibody mimic arrays (Phylos, Inc., (Lexington, Mass.)), (Affibody A B, (Bromma, Sweden)). For a more detailed treatment of antibody mimics, see Högbom et al., 2003; Nord et al., 1997 and Ronnmark et al., 2002.

As defined hereinafter, a “binding partner” is a protein which is bound by a particular protein-capture agent, preferably in a substantially specific manner. In preferred embodiments, a binding partner is one or more proteins selected from the group consisting of human IGF-1, human GMF-β, human CRMP2, human PCTAIRE-3, human HCNP, human hydroxysteroid sulfotransferase, human pyruvate dehydrogenase-E1, human antioxidant protein-2 and human DDAH-1. In certain embodiments, the binding partner is a protein set forth above, on which the protein-capture agent was selected (through in vitro or in vivo selection) or raised (as in the case of antibodies). A binding partner may be shared by more than one protein-capture agent. For instance, a binding partner which is bound by a variety of polyclonal antibodies may bear a number of different epitopes. One protein-capture agent may also bind to a multitude of binding partners, for instance, if the binding partners share the same epitope.

As defined hereinafter, an “array” is an arrangement of entities in a pattern on a substrate. Although the pattern is typically a two-dimensional pattern, the pattern may also be a three-dimensional pattern. Typically, the arrays comprise micrometer-scale, two-dimensional patterns of patches of protein-capture agents immobilized on the substrate. “Conditions suitable for protein binding” means those conditions (in terms of salt concentration, pH, detergent, protein concentration, temperature, etc.) which allow for binding to occur between an immobilized protein-capture agent and its binding partner in solution. Preferably, the conditions are not so lenient that a significant amount of nonspecific protein binding occurs. Methods for preparing arrays, array substrates and protein-capture agents are described in detail in U.S. Pat. No. 6,365,418, U.S. Pat. No. 6,475,808 and U.S. Pat. No. 6,475,809 (each specifically incorporated herein by reference in its entirety).

Typically, only one type of protein-capture agent is present on a single patch of the array. If more than one type of protein-capture agent is present on a single patch, all of the protein-capture agents of that patch must share a common binding partner. For instance, a patch may comprise a variety of polyclonal antibodies to the same antigen (although, potentially, the antibodies may bind different epitopes on that same antigen). The arrays of the invention can have any number of a plurality of different protein-capture agents.

In a preferred embodiment, the protein-capture agents are proteins. In a particularly preferred embodiment, the protein-capture agents are antibodies or antibody fragments. The antibodies or antibody fragments of the array may optionally be single-chain Fvs, Fab fragments, Fab′ fragments, F(ab′).sub.2 fragments, Fv fragments, dsFvs diabodies, Fd fragments, full-length, antigen-specific polyclonal antibodies, or full-length monoclonal antibodies. In a preferred embodiment, the protein-capture agents of the array are monoclonal antibodies, Fab fragments or single-chain Fvs.

In certain embodiments, the antibodies or antibody fragments are monoclonal antibodies against human IGF-1, human GMF-β, human CRMP2, human PCTAIRE-3, human HCNP, human hydroxysteroid sulfotransferase, human pyruvate dehydrogenase-E1, human antioxidant protein-2 and/or human DDAH-1 proteins. Alternatively, the antibody fragments are derived by selection from a library using the phage display method.

Upon using the array of protein-capture agents to bind a plurality of expression products, or fragments thereof, an array of bound proteins is created. Thus, one embodiment of the invention provides an array of bound proteins which comprises (a) a protein-capture agent array of the invention and (b) a plurality of different proteins which are expression products, or fragments thereof, of a cell, a population of cells or a biological sample obtained from a human subject, wherein each of the different proteins is bound to a protein-capture agent on a separate patch of the array. Preferably, each of the different proteins is non-covalently bound to a protein-capture agent.

Once the proteins of the biological sample have been allowed to interact with and become immobilized on the patches of the array comprising protein-capture agents with the appropriate biological specificity, the presence and/or amount of protein bound at each patch is then determined.

Use of one of the protein-capture agent arrays of the invention may optionally involve placing the two-dimensional array in a flow chamber with approximately 1-10 microliters of fluid volume per 25 mm² overall surface area. The cover over the array in the flow chamber is preferably transparent or translucent.

Alternatively, protein-containing fluid can be delivered to each of the patches of the array individually. For instance, in one embodiment, the regions of the substrate surface may be micro-fabricated in such a way as to allow integration of the array with a number of fluid delivery channels oriented perpendicular to the array surface, each one of the delivery channels terminating at the site of an individual protein-capture agent-coated patch.

The sample which is delivered to the array will typically be a fluid. In a preferred embodiment of the invention, the sample is a cellular extract or a body fluid. The sample to be assayed may optionally comprise a complex mixture of proteins, including a multitude of proteins which are not binding partners of the protein-capture agents of the array. If the proteins to be analyzed in the sample are membrane proteins, then those proteins will typically need to be solubilized prior to administration of the sample to the array. If the proteins to be assayed in the sample are proteins secreted by a population of cells in an organism, a sample which is derived from a body fluid is preferred. If the proteins to be assayed in the sample are intracellular, a sample which is a cellular extract is preferred.

In general, delivery of solutions containing proteome to be bound by the protein-capture agents of the array may optionally be preceded, followed, or accompanied by delivery of a blocking solution. A blocking solution contains protein or another moiety which will adhere to sites of non-specific binding on the array. For instance, solutions of bovine serum albumin or milk may be used as blocking solutions.

A wide range of detection methods is applicable to the methods set forth supra. As desired, detection may be either quantitative or qualitative. The invention array can be interfaced with optical detection methods such as absorption in the visible or infrared range, chemiluminescence, and fluorescence (including lifetime, polarization (or anisotropy), fluorescence correlation spectroscopy (FCS), and fluorescence-resonance energy transfer (FRET)). Furthermore, other modes of detection such as those based on optical wave guides (International Application WO 96/26432 and U.S. Pat. No. 5,677,196), surface plasmon resonance, surface charge sensors, and surface force sensors are compatible with many embodiments of the invention. Alternatively, technologies such as those based on Brewster Angle microscopy (BAM) (Schaaf et al., Langmuir, 3:1131-1135 (1987)) and ellipsometry (U.S. Pat. Nos. 5,141,311 and 5,116,121; Kim, Macromolecules, 22:2682-2685 (1984)) could be applied. Quartz crystal microbalances and desorption processes (see, e.g., U.S. Pat. No. 5,719,060) provide still other alternative detection means suitable for at least some embodiments of the invention array. An example of an optical biosensor system compatible both with some arrays of the present invention and a variety of non-label detection principles including surface plasmon resonance, total internal reflection fluorescence (TIRF), Brewster Angle microscopy, optical wave guide light mode spectroscopy (OWLS), surface charge measurements, and ellipsometry can be found in U.S. Pat. No. 5,313,264.

In other embodiments, traditional immunoassays detection techniques are used. These techniques include noncompetitive immunoassays, competitive immunoassays, and dual label, ratio-metric immunoassays. These particular techniques are primarily suitable for use with the arrays of protein-capture agents when the number of different protein-capture agents with different specificity is small (less than about 100). Many different labeling methods may be used in the aforementioned techniques, including radioisotopic, enzymatic, chemiluminescent, and fluorescent methods.

Finally, U.S. Pat. No. 6,534,270, specifically incorporated herein by reference in its entirety, describes a novel method for fabricating an array or “biochip”. Briefly, the method for fabricating biochips includes the steps of: (a) immersing fibers wound on solid supports in a solution containing biomolecules to absorb and immobilize the biomolecules onto the fibers; (b) arranging the individual fibers with the biomolecules immobilized thereon, the fibers being separated from each other at a predetermined distance; (c) embedding the arranged fibers with a defined material; (d) cutting the embedded fibers in a direction perpendicular to the lengthwise arrangement direction of the fibers to obtain thin chips; and (e) placing the chips on a substrate and removing the defined material used to embed the fibers, thereby leaving the fiber fragments with the immobilized biomolecules on the substrate. The fibers are embedded with a wax, ice, or polymer material, and the substrate is a solid substrate.

3. Gene Delivery

In certain embodiments, the invention is directed to a method for the treatment of a mood disorder in a human subject in need thereof. In one particular embodiment, a method for the treatment of a mood disorder comprises delivering one or more polynucleotides encoding one or more wild-type polypeptides selected from the group consisting of human IGF-1, a human GMF-β, a human CRMP2, a human PCTAIRE-3, a human HCNP, a human hydroxysteroid sulfotransferase, a human pyruvate dehydrogenase-E1, a human antioxidant protein-2 and a human DDAH-1.

Thus, in certain embodiments, the present invention is directed to methods of screening a human subject for a mood disorder. In particular embodiments, a method of screening a human subject for a mood disorder comprises determining the expression level(s) of one or more polypeptides selected from the group consisting human IGF-1, a human GMF-β, a human CRMP2, a human PCTAIRE-3, a human HCNP, a human hydroxysteroid sulfotransferase, a human pyruvate dehydrogenase-E1, a human antioxidant protein-2 and a human DDAH-1, wherein lower polypeptide levels in the subject indicate a mood disorder. Alternatively, in other embodiments, a method of screening a human subject for a mood disorder comprises determining the expression level(s) of one or more mRNA(s) selected from the group consisting human IGF-1, a human GMF-β, a human CRMP2, a human PCTAIRE-3, a human HCNP, a human hydroxysteroid sulfotransferase, a human pyruvate dehydrogenase-E1, a human antioxidant protein-2 and a human DDAH-1, wherein lower polypeptide levels in the subject indicate a mood disorder.

In a preferred embodiment, a subject identified above as having a mood disorder via one or more of the screening methods of the invention, is administered a wild-type polynucleotide encoding the one or more polypeptides identified as being expressed at low levels. Thus, in particular embodiments, the present invention is directed to gene therapy methods to treat a mood disorder.

An IGF-1, GMF-β, CRMP2, PCTAIRE-3, HCNP, hydroxysteroid sulfotransferase, pyruvate dehydrogenase, antioxidant protein-2 or DDAH-1 polypeptide of the invention, in addition to the gene delivery methods described below, is also delivered systemically or locally in humans for therapeutic benefits. Barrier devices, which contain transfected cells expressing a therapeutic polypeptide product and through which the therapeutic product is freely permeable, can be used to retain cells in a fixed position in vivo or to protect and isolate the cells from the host's immune system. Barrier devices are particularly useful and allow transfected immortalized cells, transfected cells from another species (transfected xenogeneic cells), or cells from a nonhistocompatibility-matched donor (transfected allogeneic cells) to be implanted for treatment of a human. Barrier devices also allow convenient short-term (i.e., transient) therapy by providing ready access to the cells for removal when the treatment regimen is to be halted for any reason. Transfected xenogeneic and allogeneic cells may be used for short-term gene therapy, such that the gene product produced by the cells will be delivered in vivo until the cells are rejected by the host's immune system.

Gene delivery (i.e., gene therapy) is a promising method for the treatment of acquired and inherited disease, and a number of viral based systems for gene transfer purposes have been described. For example, retroviral systems are currently the most widely used viral vector systems for gene transfer. For descriptions of various retroviral systems, see, e.g., U.S. Pat. No. 5,219,740; Miller and Rosman, 1989; Miller, 1990; Scarpa et al., 1991; Burns et al., 1993 and Boris-Lawrie and Temin, 1993.

A number of adenovirus based gene delivery systems have also been developed. Human adenoviruses are double-stranded DNA viruses which enter cells by receptor-mediated endocytosis. These viruses are particularly well suited for gene transfer because they are easy to grow and manipulate, and they exhibit a broad host range in vivo and in vitro. Adenovirus is easily produced at high titers and is stable so that it can be purified and stored. Even in the replication-competent form, adenoviruses generally cause only low level morbidity and are not associated with human malignancies. For descriptions of various adenovirus-based gene delivery systems, see, e.g., Haj-Ahmad and Graham, 1986; Bett et al., 1993; Mittereder et al., 1994; Seth et al., 1994; Barr et al., 1994; Berkner, 1988; Rich et al., 1993.

The use of adeno-associated virus (MV) gene delivery systems have been developed. Recombinant vectors based on MV particles have been used for DNA delivery. AAV is a helper-dependent DNA parvovirus which belongs to the genus Dependovirus. AAV has a wide host range and is able to replicate in cells from any species so long as there is also a successful infection of such cells with a suitable helper virus. MV has not been associated with any human or animal disease. For a review of MV, see, e.g., Berns and Bohenzky, 1987. The construction of recombinant vectors based on MV has been described (see, e.g., U.S. Pat. Nos. 6,531,456, 5,173,414 and 5,139,941 and International Application Nos. WO 92/01070 and WO 93/03769, each incorporated by reference in its entirety).

Recombinant AAV vectors are capable of transducing several cell types, including hematopoietic cells, respiratory epithelial cells (Flotte et al., 1992; Flotte et al., 1993(a); Flotte et al., 1993(b)) and neurons of the central nervous system (Kaplitt et al., 1994). These cell types are well-differentiated, slowly-dividing or postmitotic. A recombinant MV-based gene transfer system has been described for the transduction of HSV-tk into cells of the central or peripheral nervous systems in a mammalian subject to render those cells sensitive to ganciclovir (see, International Application No. WO 95/28493). This system is particularly designed for use in the treatment of neurological disorders such as Parkinson's disease and in the treatment of brain tumors.

Lentiviruses are also used as gene therapy vectors. In addition to the long-term expression of the transgene provided by all retroviral vectors, lentiviruses present the opportunity to transduce non-dividing cells and potentially achieve regulated expression (U.S. Pat. No. 6,506,378, incorporated by reference herein in its entirety). Other viral vectors employed as expression constructs in the present invention include vectors derived from viruses such as vaccinia virus, Moloney murine leukemia virus (MMLV); VSV-G type retroviruses (U.S. Pat. No. 5,817,491), papovaviruses such as JC, SV40, polyoma (U.S. Pat. No. 5,624,820), Epstein-Barr Virus (EBV), papilloma viruses (U.S. Pat. No. 5,674,703), and more particularly, bovine papilloma virus type I (BPV; U.S. Pat. No. 4,419,446), poliovirus and herpesviruses.

The transfer of an expression construct into cultured mammalian cells (e.g., ex vivo of gene delivery) is described in U.S. Pat. No. 6,506,378, incorporated by reference herein in its entirety. This method describes a treatment for Parkinson's disease via increasing the efficiency of L-DOPA to dopamine conversion in a mammal. Briefly, the method comprises (a) obtaining cells from a mammal; (b) transforming the cells in vitro with a first polynucleotide encoding L-amino acid decarboxylase (MDC) and a second polynucleotide encoding vesicular monoamine transporter (VMAT), wherein the polynucleotides each are under transcriptional control of a promoter; (c) implanting the transformed cells into the mammal; and (d) providing L-DOPA to the mammal, whereby AADC converts L-DOPA in vivo to dopamine and VMAT sequesters the dopamine in endosomes of said cells, which sequestered dopamine releases over a longer duration of time than from cells without storage of L-DOPA.

In certain embodiments, the delivery of a gene encoding a protein of the present invention is provided by implanting donor cells expressing the gene of interest. The choice of the donor cells for implantation depends heavily on the nature of the expressed gene, characteristics of the vector and the desired phenotypic result. For example, retroviral vectors require cell division and DNA synthesis for efficient infection, integration and gene expression, if such vectors are used, the donor cells are preferably actively growing cells, such as primary fibroblast culture or established cell lines, replicating embryonic neuronal cells or replicating adult neuronal cells from selected areas such as the olfactory mucosa and possibly developing or reactive glia.

Primary cells (i.e., cells that have been freshly obtained from a subject), such as fibroblasts, that are not in the transformed state are preferred for use in the present invention. Other suitable donor cells include immortalized (transformed cells that continue to divide) fibroblasts, glial cells, adrenal cells, hippocampal cells, keratinocytes, hepatocytes, connective tissue cells, ependymal cells, bone marrow cells, stem cells, leukocytes, chromaffin cells and other mammalian cells susceptible to genetic manipulation and grafting using the methods of the present invention. Additional characteristics of donor cells which are relevant to successful grafting include the age of the donor cells.

Furthermore, there are available methods to induce a state of susceptibility in stationary, non-replicating target cells that will allow many other cell types to be suitable targets for viral transduction. For instance, methods have been developed that permit the successful viral vector infection of primary cultures of adult rat hepatocytes, ordinarily refractory to infection with such vectors, and similar methods may be helpful for a number of other cells (Wolff et al., 1987). In addition, the development of many other kinds of vectors derived from herpes, vaccinia, adenovirus, or other viruses, as well as the use of efficient non-viral methods for introducing DNA into donor cells such as electroporation, lipofection or direct gene insertion may be used for gene transfer into many other cells.

In certain embodiments, gene expression is regulated by an inducible expression system such as the ecdysone system (Invitrogen, Carlsbad, Calif.) or the Tet-Off™ or Tet-On™ system (Clontech, Palo Alto, Calif.). The ecdysone system is designed to allow regulated expression of a gene of interest in mammalian cells. It consists of a tightly regulated expression mechanism that allows virtually no basal level expression of the transgene, but over 200-fold inducibility. The system is based on the heterodimeric ecdysone receptor of Drosophila, and when ecdysone or an analog such as muristerone A binds to the receptor, the receptor activates a promoter to turn on expression of the downstream transgene and high levels of mRNA transcripts are attained. In this system, both monomers of the heterodimeric receptor are constitutively expressed from one vector, whereas the ecdysone-responsive promoter which drives expression of the gene of interest is on another plasmid.

The Tet-Off™ or Tet-On™ allows high levels of gene expression to be regulated in response to tetracycline or tetracycline derivatives such as doxycycline. In the Tet-On™ system, gene expression is turned on in the presence of doxycycline, whereas in the Tet-Off™ system, gene expression is turned on in the absence of doxycycline. These systems are based on two regulatory elements derived from the tetracycline resistance operon of E. coli. The tetracycline operator sequence to which the tetracycline repressor binds, and the tetracycline repressor protein. The gene of interest is cloned into a plasmid behind a promoter that has tetracycline-responsive elements present in it. A second plasmid contains a regulatory element called the tetracycline-controlled transactivator, which is composed, in the Tet-Off™ system, of the VP16 domain from the herpes simplex virus and the wild-type tetracycline repressor. Thus in the absence of doxycycline, transcription is constitutively on. In the Tet-On™ system, the tetracycline repressor is not wild type and in the presence of doxycycline activates transcription. For gene therapy vector production, the Tet-Off™ system is preferable so that the producer cells are grown in the presence of tetracycline or doxycycline and prevent expression of a potentially toxic transgene, but when the vector is introduced to the patient, the gene expression would be constitutively on.

In certain embodiments, viral promoters with varying strengths of activity are utilized depending on the level of expression desired. In mammalian cells, the CMV immediate early promoter is often used to provide strong transcriptional activation. Modified versions of the CMV promoter that are less potent are also used when reduced levels of expression of the transgene are desired. When expression of a transgene in hematopoietic cells is desired, retroviral promoters such as the LTRs from MLV or MMTV are often used. Other viral promoters that are used include SV40, RSV LTR, HIV-1 and HIV-2 LTR, adenovirus promoters such as from the E1A, E2A, or MLP region, AAV LTR, cauliflower mosaic virus, HSV-TK, and avian sarcoma virus.

Similarly tissue specific promoters are used to effect transcription in specific tissues or cells so as to reduce potential toxicity or undesirable effects to non-targeted tissues. In certain indications, it is desirable to activate transcription at specific times after administration of the gene therapy vector. This is done with promoters that are hormone or cytokine regulatable.

Thus, an expression construct of the invention is delivered to a human host, or human cells obtained from the host, via a viral vector as described supra. Additionally, several non-viral methods for the transfer of expression constructs into cultured mammalian cells also are contemplated by the present invention. These include calcium phosphate precipitation, DEAE-dextran, electroporation, direct microinjection, DNA-loaded liposomes and lipofectamine-DNA complexes, cell sonication, gene bombardment using high velocity micro-projectiles and receptor-mediated transfection.

U.S. Pat. No. 6,436,708, incorporated herein by reference in its entirety, describes a method for increasing gene expression in cells of the central nervous system comprising administering at (or adjacent to) cells of the central nervous system tissue a polycation (lipid associated)-condensed nucleic acid, wherein the nucleic acid encodes a protein associated with a genetic disorder and the cation lipid associated polycation condensed nucleic acid enhances gene expression in the cells of the central nervous system.

In certain embodiments, gene transfer may more easily be performed under ex vivo conditions. Ex vivo gene therapy refers to the isolation of cells from an animal, the delivery of a polynucleotide into the cells in vitro, and then the return of the modified cells back into an animal. This may involve the surgical removal of tissue/organs from an animal or the primary culture of cells and tissues. U.S. Pat. Nos. 6,440,407; 5,922,597 and 5,766,920, each incorporated herein by reference in its entirety, describe various methods for ex vivo manipulation of cells.

4. Screening Monoamine Re-Uptake Inhibitors

In certain embodiments, the present invention is directed to methods for screening and identifying compositions which inhibit monoamine re-uptake in vivo and in vitro. For example, in certain embodiments, the invention is directed to an in vivo method for monitoring the kinetics of monoamine re-uptake inhibitors in rodents, as described in Example 2. In certain other embodiments, the invention is directed to in vitro methods (e.g., recombinant cell lines) for monitoring the kinetics of monoamine re-uptake inhibitors (e.g., see Example 3).

Monoamine re-uptake inhibitors, such as fluoxetine and venlafaxine, exhibit slow response kinetics with regard to attenuation of mood disorder symptoms. For example, a patient receiving a fluoxetine treatment regimen typically requires about two weeks for an attenuation in depressive symptoms. Thus, the methods set forth in Examples 2 and 3 provide a means for monitoring the kinetics of monoamine re-uptake inhibitors in vivo and in vitro, respectively.

As defined hereinafter, a “compound” or “composition” refers to any molecule which inhibits monoamine re-uptake in vivo or in vitro. Examples of compounds screened according to the methods of the invention include, but are not limited to, molecules such as proteins, peptide fragments, amino acids, peptide mimetics, antibodies, antibody fragments, antisense oligonucleotides, small organic molecules, metal chelates, ions or any combination thereof.

Thus, in certain embodiments, the invention is directed to in vivo methods for monitoring the kinetics of known antidepressants (e.g., venlafaxine or fluoxetine) by detecting modulation of the expression levels of one or more polypeptides selected from the group consisting of IGF-1, GMF-β, CRMP2, PCTAIRE-3, HCNP, hydroxysteroid sulfotransferase, pyruvate dehydrogenase, antioxidant protein-2 and DDAH-1. For example, the method generally comprises administering to a plurality of rodents either a monoamine re-uptake inhibitor or a placebo. Then, at a desired time point, a hippocampus is obtained from one of the plurality of rodents administered the monoamine re-uptake inhibitor and a hippocampus is obtained from one of the plurality of rodents administered a placebo. The amount of one or more proteins in the hippocampus is then determined, wherein the one or more proteins are selected from the group consisting of IGF-1, GMF-β, CRMP2, PCTAIRE-3, HCNP, hydroxysteroid sulfotransferase, pyruvate dehydrogenase, antioxidant protein-2 and DDAH-1. The above steps are repeated to cover a range of time points over a desired time course. For example, a range of desired time points are gathered from 0 days to about 36 days, and the expression levels of one or more proteins obtained from a hippocampal extract monitored. In certain preferred embodiments, this method is used to screen compound libraries for compounds which modulate the expression levels of one or more polypeptides selected from the group consisting of IGF-1, GMF-β, CRMP2, PCTAIRE-3, HCNP, hydroxysteroid sulfotransferase, pyruvate dehydrogenase, antioxidant protein-2 and DDAH-1.

In other embodiments, a transgenic animal such as a rodent (e.g., see Section E) is genetically modified to under- or over-express one or more polypeptides selected from the group consisting of IGF-1, GMF-β, CRMP2, PCTAIRE-3, HCNP, hydroxysteroid sulfotransferase, pyruvate dehydrogenase, antioxidant protein-2 and DDAH-1. Subsequently, these animals are administered a test compound and changes in mRNA or protein levels are measured.

In certain other embodiments, the invention provides recombinant cell lines for screening compounds which modulate the expression of one or more polypeptides selected from the group consisting of IGF-1, GMF-β, CRMP2, PCTAIRE-3, HCNP, hydroxysteroid sulfotransferase, pyruvate dehydrogenase, antioxidant protein-2 and DDAH-1. U.S. Pat. No. 6,475,725, incorporated herein by reference in its entirety, describes recombinant CHO clones which are stable for at least 40 generations in serum free and protein-free medium. U.S. Pat. No. 5,869,463, specifically incorporated herein by reference in its entirety, describes immortalized human fetal cells derived from cells of the central nervous system and U.S. Pat. No. 6,383,805, incorporated herein by reference in its entirety, describes epithelial cell cultures for in vitro testing.

Thus, test compounds of the invention can be administered to cells genetically engineered to express (or endogenously expressing) one or more proteins selected from the group consisting of IGF-1, GMF-β, CRMP2, PCTAIRE-3, HCNP, hydroxysteroid sulfotransferase, pyruvate dehydrogenase, antioxidant protein-2 and DDAH-1; or recombinant cells such as described above which are transfected with a polynucleotide encoding a polypeptide selected from the group consisting of IGF-1, GMF-β, CRMP2, PCTAIRE-3, HCNP, hydroxysteroid sulfotransferase, pyruvate dehydrogenase, antioxidant protein-2 and DDAH-1.

### G. EXAMPLES

The following examples are carried out using standard techniques, which are well known and routine to those of skill in the art, except where otherwise described in detail. The following examples are presented for illustrative purpose, and should not be construed in any way as limiting the scope of this invention.

### Example 1

### Proteomic Analysis of Protein Changes Developing in rat Hippocampus After Chronic Antidepressant Treatment

Materials and Methods

Animal dosing. Adult male Sprague Dawley rats weighing 230-320 g were housed with ad libitum access to food and water. After 1 week of habituation, rats were administered either an antidepressant or vehicle alone. The following drugs were administered intraperitoneally (i.p.): fluoxetine (10 mg/kg daily), venlafaxine (10 mg/kg daily) or vehicle alone (1 ml/kg normal saline daily) for 14 days (n=3 rats per group).

Sample preparation and cytosolic protein extraction. The animals were euthanized and hippocampi were rapidly dissected from the whole brain. Samples from each treatment group were pooled and stored at −85° C. Cytosolic extracts were prepared using the universal extraction kit from Sigma (prot-two) as follows: soluble cytoplasmic extraction reagent (S2688) was added to the hippocampal tissue (10 ml/250 mg wet weight). Samples were sonicated (4 times at 30 seconds) and centrifuged for 45 minutes (14,000 g) at 4° C. The supernatant was decanted and stored on ice. Ten milliliters of the soluble cytoplasmic extraction reagent was added to the remaining pellet and the sonication and centrifugation steps were repeated. The supernatant was decanted, combined with the previous supernatant and SpeedVac (Savant Instruments, Halbrook, N.Y.) dried. Each sample was dissolved in 2 ml of 2-D lysis buffer (6 M urea, 2 M thiourea, 4% CHAPS, 40 mM Tris, 65 mM dithiothreitol (DTT), 1 mM sodium vanadate, 1 mM sodium fluoride, protease inhibitor tablet (#1836145, Roche)), concentrated and desalted (Centricon tube; 6500 rpm) at 10° C. for 12 hours.

Two-dimensional gel electrophoresis. First dimensional isoelectric focusing (IEF) was performed on 18 cm IPG strips (pH 4-7) using an IPGphor unit (Amersham Pharmacia). Each strip was rehydrated for 16 hours with sample lysate (˜500 ug) in a final volume of 400 uL of IEF solution (10 uL of bromophenol blue (0.25% w/v); 2 uL of Ampholyte (pH 4-7, Amersham #17-6000-86); 2-D lysis buffer). IEF was then carried out using the following conditions: focusing started at (i) 500 V, 500 Vh; the voltage was gradually ramped (ii) 1000 V, 1000 Vh; and kept constant (iii) 5,000 V, 105,000 Vh; finally reduced (iv) 100 V, 200 Vh. The strips were then subjected to a two-step equilibration (50 mM Tris; 2% SDS; 30% glycerol) in (A) 0.5% DTT (Bio-Rad) and (B) 4.5% iodoacetamide (Sigma) buffers before proceeding to SDS-PAGE. Separation in the second dimension was performed on 10% SDS polyacrylamide gels (180×1.5 mm) at a constant voltage of 500 V at 15° C. for 28 hours using an Investigator 2D electrophoresis unit (Genomic Solutions Inc., MA) which accommodated 10 gels. Molecular masses were determined by running standard protein markers (Genomic Solutions Inc.), covering the range 10-200 kDa as well as internal verification via landmark proteins within the gel. The pl values were used as given by the supplier of the immobilized pH-gradient strips.

Protein fixation, silver staining and 2-D gel comparisons. Silver staining of the gels was performed using a silver stain kit (gs#80-0183; Genomic Solutions). All steps were performed at room temperature, gently agitating the trays on a rotary shaker at low speed. Stained gels were scanned using an 8-bit ccd camera and the Investigator G3 ProPic system (Genomic Solutions Inc.). Protein spots were outlined (first automatically and then manually) and quantified using the HT analyzer v2.2 software (Genomic Solutions Inc.). The software calculated the relative spot volume of the proteins compared with the total amount of protein in the gel. The HT analyzer software was used to cross-match (‘synchronize’) and identify protein spots between control gels and drug treatment gels that were different in integrated intensity by at least a factor of 1.5 versus control. This population of protein spots was then re-analyzed to determine the subset of spots that cross-matched within the antidepressant-treated gels subdivided as either up-regulated or down-regulated. This population of spots were automatically picked from each gel with the ProPic system for in-gel trypsin digestion.

Enzymatic digestion of protein spots. Silver-stained protein spots were automatically excised and transferred to a 96-well polypropylene microtiter plate. Each excised spot was de-stained to remove the silver ions and enhance sensitivity with 30 mM potassium ferricyanide and 100 mM sodium thiosulfate (1:1 ratio). The gel spots were covered with the de-staining solution for 20 minutes, or until the brownish color disappeared. Each excised spot was washed with 200 uL of 10% methanol for 10 minutes. Two hundred uL of 50 mM NH₄HCO₃ in 50% acetonitrile was added to each tube (shaking for 10 minutes) so as to shrink the gel. The liquid was discarded and the previous step repeated once with a fresh aliquot of 50 mM NH₄HCO₃ in 50% acetonitrile. The gel pieces were re-swelled in 200 uL reduction buffer (10 mM DTT in 50 mM NH₄HCO₃) and incubated for 30 minutes at 60° C. The gel samples were spun down (5000 rpm. for 2 minutes) and the liquid was aspirated. This step was critical for the washing of gel spots since it allowed for more efficient enzyme digestion and removal of SDS. The above wash and dehydration steps were repeated once more. The final washing solution was then aspirated and 200 uL alkylation buffer (55 mM iodoacetamide in 50 mM NH₄HCO₃) was added and incubated for 20 minutes (in the dark) at room temperature. The liquid was discarded and gel pieces washed 3 times with 50 mM NH₄HCO₃ in 50% acetonitrile, prior to drying in a SpeedVac evaporator. The dried gel pieces were rehydrated with 40 uL of 1 ug/uL sequencing-grade modified trypsin (Promega) in 50 mM NH₄HCO₃ plus 5 mM CaCl₂ and incubated at 37° C. for 6-8 hours with gentle shaking. To enhance peptide extraction, the tubes were sonicated for 10 minutes at 37° C. in an ultrasonic water bath (Crest Ultrasonics, Trenton, N.J.) and spun at 5000 rpm for 2 minutes. The supernatant was transferred to a clean tube and 100 uL acetonitrile was added to the gel piece followed by sonication (15 minutes) at 37° C. and centrifugation (5000 rpm for 2 minutes). The gel-peptide extraction process was repeated a further 3 times with 100 uL of 5% formic acid, acetonitrile:formic acid (3:1) and acetonitrile:formic acid (1:1) followed by sonication and centrifugation after each step as described above. All supernatant extracts were combined for each gel piece. The samples were lyophilized and stored.

MALDI-TOF-Mass Spectrometric (MS) Analysis of tryptic peptide digests. After Zip-tip clean-up, mass analyses of the individual samples were performed using a PerSeptive Biosystems Voyager-DE STR matrix-assisted laser desorption/ionization time-of-flight mass spectrometer (MALDI-TOF-MS) (Framingham, Mass.). Peptide mixture (0.7 uL in 0.1% TFA, 20% acetonitrile) was applied with an equal volume of matrix, (CHCA (10 mg/ml) dissolved in 50% acetonitrile/0.1% TFA). Matrix concentrations ranged from 2 mg/ml to 10 mg/ml according to the amount of peptide. Standard calibration peptides (des-Arg-Bradykinin, Angiotensin1, Glu-Fibrinopeptide B) (Applied Biosystems) were also applied to the MALDI plate. For each sample, spectra were acquired in the delayed extraction and reflector mode and an average of 150 spectra that passed the accepted criterion of peak intensity were automatically selected and saved. Spectra were automatically calibrated upon acquisition using a 3-point calibration set-up. Processing (noise reduction/smoothing, baseline correction, de-isotoping) and assignment of peaks was done automatically with the PS-1 system from Applied Biosystems. Measured peptide masses were excluded if their masses corresponded to trypsin autodigestion products.

Database searching and identification of proteins. Peptide matching and protein searches were performed automatically in SWISS-PROT and NCBI non-redundant databases with the use of MS-Fit (Protein Inspector; UCSF, San Francisco, Calif.). Peptide masses were compared with theoretical values for all available proteins from all species. Monoisotopic masses were used, and a mass tolerance of 0.0025% was allowed. Unmatched peptides or miscleavage sites were not considered. All mass searches were performed using a mass window between 1 kDa and 100 kDa and included rat sequences. The search parameters allowed for oxidation of methionine, N-terminal acetylation, carboxyamidomethylation of cysteine and phosphorylation of serine, threonine and tyrosine. The criteria for positive identification of proteins were set as follows: (i) the MS match consisted of a minimum of 4 peptides; (ii) 50 ppm or better mass accuracy; (iii) the matched peptides covered at least 15% of the whole protein sequence; (iv) the molecular weight and pl of identified proteins matched estimated values obtained from image analysis or from published 2D databases and (v) the protein exhibited a significant difference in the number of matched peptides to the next potential hit.

Antidepressant treatment and BrdU dosing. Dosing of adult male Sprague Dawley rats was performed as described above for 14 days (n=10 rats per group). To evaluate the effect of chronic antidepressant treatment on proliferation of progenitor stem cells, 24 hours after the last antidepressant injection, rats were administered BrdU (Sigma, St. Louis, Mo.) (50 mg/kg rig ip injection twice a day) for 4 days prior to euthanesia. To evaluate the effect of chronic antidepressant treatment on survival of progenitor stem cells, a sub-group of rats were allowed to live for 4 weeks after BrdU injection. All rats were perfused through the ascending aorta with 50 ml of 0.9% saline, followed by 400 ml of freshly prepared 4% paraformaldehyde in PBS (pH 7.5). After 30-60 minutes post-perfusion, rat brains were removed and postfixed in 4% paraformaldehyde in PBS overnight, followed by immersion in 30% sucrose/PBS solution for 48-72 hours at 4° C. Brains were rapidly frozen on dry-ice and stored at −80° C. Brain sectioning and staining for BrdU immunoreactivity were performed as previously described by Malberg et al. (2000). Briefly, serial coronal sections of each brain were cut (35 μm thickness) through the entire hippocampus (stereotaxic plates 26-40 according to Paxinos, Watson and Emson, 1980) using a freezing microtome. Every sixth section throughout the hippocampus was processed for BrdU staining. Free-floating sections were maintained in 100 mM PBS (about 100 sections per brain). DNA denaturation of the sections was performed as follows: sections were treated with 50% formamide/50% 2×SSC (0.3 M NaCl/0.03 M sodium citrate) for 2 hours at 65° C., rinsed in PBS (2 times for 5 minutes at room temperature), incubated with 2 N HCl for 30 minutes, followed by 0.1 M boric acid buffer (pH 8.5) for 10 minutes and a final rinse in PBS. For BrdU immunostaining, sections were incubated with 3% H₂O₂ in PBS for 30 minutes, rinsed in PBS and blocked with 3% normal horse serum in 0.01% TritonX-100 for 30 minutes. Anti-mouse BrdU (1:250 Sigma) was applied to the free-floating sections and incubated overnight at 4° C. The sections were rinsed in PBS, incubated with biotinylated horse anti-mouse IgG (Vector Lab) for 30 minutes and washed in PBS. ABC reagent complex (Vectastain Elite kit from Vector) was added (as per manufacturer's instructions) for 30 minutes, followed by rinsing in PBS and color development with DAB solution for 4 minutes. Sections were mounted on glass slides and coverslipped. All BrdU labeled cells in the subgranular zone (SGZ) were counted in each section by an experimenter blinded to the study code. All counts were performed at 200× magnification using a light microscope (Zeiss Axiovert 135). The total number of BrdU labeled cells per section were determined and multiplied by 12 to obtain the total number of cells per dentate gyrus. Comparative analyses between treatments were performed using a Tukey HSD post hoc analysis for multiple comparisons.

Results

The changes in the proteome that occurred after long-term exposure of rats to two clinically effective antidepressant drugs, fluoxetine (a selective serotonin re-uptake inhibitor) and venlafaxine (a dual serotonin/norepinephrine re-uptake inhibitor) were determined. Hippocampal cytosolic extracts were isolated from control (untreated) and venlafaxine-treated or fluoxetine-treated rats and analyzed by two-dimensional electrophoresis (2-DE). To optimize proteome analysis of rat hippocampal proteins, commercially available nonlinear immobilized pH gradient (IPG) strips were used (18 cm, pH 4-7), rather than a pH gradient of pH 3-10. Initial studies indicated that this pH range covers most of the cytosolic proteins in their soluble form and provided higher gel-gel comparative resolution and probability of successful protein spot identification by mass spectrometry. Protein separation was consistently better in the lower molecular weight range and toward the neutral pH. Horizontal streaks were apparent for proteins in the higher molecular weight range, which is a common feature in most 2-DE gels. The digitized master gel was composed of control (untreated) 2-DE gels plus those originating from fluoxetine-treated and venlafaxine-treated sample gels. The master gel revealed an average of 545 spots for the controls, 664 spots for fluoxetine-treated and 729 spots for venlafaxine-treated.

Comparing the 2-DE patterns of the untreated versus the fluoxetine-treated and venlafaxine-treated hippocampal samples, the intensity of the majority of the silver-stained spots remained unchanged. These landmarks were used as reference points for highlighting the population of protein spots that were altered in their silver-staining intensities. Only protein spots that occurred in all 2-DE gels from this study were analyzed. Those protein spots either up-regulated or down-regulated by both antidepressants versus control were selected for identification by mass spectrometric analysis. Thirty-three spots (31 up-regulated and 2 down-regulated) were identified as antidepressant modulated proteins in vivo and different in integrated intensity by at least a factor of 1.5 versus control. Due to the inherent limitations of silver staining, only qualitative differences could be documented. The identities of these proteins were established by MALDI-TOF mass spectra (MS) analyses. The MS fingerprinting criteria used to identify the proteins were as described in Materials and Methods above. The protein species identified were assigned to different categories based on documented biological function as summarized in Table 1. Several antidepressant-modulated spots produced poor MALDI-TOF mass spectra that did not permit unequivocal protein identification according to the pre-set criteria. These proteins currently have no matches for their tryptic peptide fingerprints.

It was observed that both venlafaxine and fluoxetine led to alterations in the biological protein profile (proteome) of the hippocampus after 2 weeks systemic administration. The proteins identified were mostly cytosolic and mitochondrial proteins, as well as proteins involved in structural processes, and metabolic and synthetic cellular maintenance (see Table 1). Functional protein groupings indicated an up-regulation of factors that were associated with the induction of neurogenesis (e.g., IGF-1, GMF-β) and the promotion of outgrowth/maintenance of neuronal processes (e.g., HCNP-precursor, PCTAIRE-3 and SPI-2.1) as well as anti-apoptotic activity (DDAH 1, AOP-2 and pyruvate dehydrogenase-E1) (see Table 1).

The up-regulation of hippocampal hydroxysteroid sulfotransferase A (an important neurosteroidogenic pathway enzyme) further suggests a role for antidepressants in modulating hippocampal plasticity through this pathway. Moreover, the small GTPases, Rab1a/Rab4a, which normally serve to direct vesicular trafficking and to effect synaptic plasticity, were identified to be up-regulated by antidepressant treatment. Similarly, HSP10 was also up-regulated and represents a mitochondrial molecular chaperone that mediates folding and assembly of macromolecular structures.

An overall up-regulation was observed for enzymes active in glycolysis and glucose metabolism, namely α-enolase, lactate dehydrogenase and pyruvate dehydrogenase-E1. Proteins participating in the disposal of the oxygen radicals, antioxidant protein 2 was up-regulated, whereas glutathione S-transferase Yb3 was down-regulated (Table 1). The expression of adenine phosphoribosyl transferase, contributing to the purine salvage pathway, was found to be up-regulated. Proteasome subunit α type 2, a large intracellular protease that is responsible for the majority of intracellular protein degradation, was up-regulated 10-fold by venlafaxine and 6-fold by fluoxetine. An up-regulation of several transcriptional/translational ribosomal factors was also observed after antidepressant treatment, representing an active increase in protein synthetic/modification processes at the genetic level.

In vivo studies were performed to measure the effects of antidepressant drug treatment on adult hippocampal neurogenesis by immunological methods. The method used to detect cell proliferation involved 5-bromo-2-deoxyuridine (BrdU) dosing twice a day for 4 days after a 2 week systemic administration of either saline, fluoxetine or venlafaxine. The presence of BrdU within the vicinity of dividing progenitor stem cells in the hippocampal region allowed for its incorporation and the detection of DNA synthesis (the more cell proliferation the more DNA synthesis). The number of BrdU-positive cells was visualized by immunoperoxidase staining. The BrdU immunostaining and quantitative results indicated a significant increase (˜33%) in the proliferation rate of progenitor stem cells in the subgranular zone (SGZ) of the hippocampus for both venlafaxine (P<0.01; n=5) and fluoxetine (P<0.01; n=5) versus saline controls. In comparison of these findings with BrdU immunostaining studies measuring long-term survivability of progenitor stem cells (i.e., 4 weeks after the last antidepressant dose), it was observed that the actions of venlafaxine were still prevalent with an 80% sustainability in the rate of proliferation and survivability of progenitor stem cells over basal levels. Fluoxetine treatment also demonstrated a significant preservation of proliferative and survivability effects over basal levels. The activity of venlafaxine, however, was more pronounced (−40% more cells) compared with fluoxetine under the given experimental paradigms.

### Example 2

### Monitoring Monoamine Re-Uptake Inhibitor Kinetics In Vivo

Monoamine re-uptake inhibitors, such as fluoxetine and venlafaxine, exhibit slow response kinetics with regard to attenuation of mood disorder symptoms. For example, a patient receiving a fluoxetine treatment regimen typically requires about two weeks for an attenuation in depressive symptoms. The following non-limiting example describes a method for monitoring the kinetics of monoamine re-uptake inhibitors in vivo. The method generally comprises administering to a plurality of rodents either a monoamine re-uptake inhibitor or a placebo. Then, at a desired time point, a hippocampus is obtained from one of the plurality of rodents administered the monoamine re-uptake inhibitor and a hippocampus is obtained from one of the plurality of rodents administered a placebo. The amount of one or more proteins in the hippocampus is then determined, wherein the proteins are IGF-1, GMF-β, CRMP2, PCTAIRE-3, HCNP, hydroxysteroid sulfotransferase, pyruvate dehydrogenase, antioxidant protein-2 and/or DDAH-1. The above steps are repeated to cover a range of time points over a desired time course. For example, a range of desired time points are gathered from 0 days to about 36 days, and the expression levels of one or more proteins obtained from a hippocampal extract monitored.

### Example 3

### Monitoring Monoamine Re-Uptake Inhibitor Kinetics In Vitro

The following non-limiting example describes a method for monitoring the kinetics of monoamine re-uptake inhibitors in vitro. For example, recombinant cells are used that either endogenously express or are genetically engineered to express one or more of the proteins IGF-1, GMF-β, CRMP2, PCTAIRE-3, HCNP, hydroxysteroid sulfotransferase, pyruvate dehydrogenase, antioxidant protein-2 and/or DDAH-1. A plurality of the recombinant cells are administered a monoamine re-uptake inhibitor or a placebo. Subsequently, at a desired time point, a cell from one of the plurality of cells administered the monoamine re-uptake inhibitor and a cell from one of the plurality of cells administered the placebo are obtained and the amount of one or more proteins (or mRNA) is determined. These measurements are repeated over a range of desired time points, e.g., from 0 days to about 36 days.

Equivalents: Those skilled in the art will recognize, or be able to ascertain using no more than routine experimentation, many equivalents to the specific embodiments of the invention described herein. Such equivalents are intended to be encompassed by the following claims. All patents and publications cited herein are incorporated by reference.

### REFERENCES

European Application No. EP 125023

European Application No. EP 171496

European Application No. EP 173494

European Application No. EP 184187

European Application No. EP 264166

U.S. Pat. No. 4,196,265

U.S. Pat. No. 4,419,446,

U.S. Pat. No. 4,816,567

U.S. Pat. No. 4,873,316

U.S. Pat. No. 5,116,121

U.S. Pat. No. 5,141,311

U.S. Pat. No. 5,219,740

U.S. Pat. No. 5,223,409

U.S. Pat. No. 5,258,454

U.S. Pat. No. 5,313,264

U.S. Pat. No. 5,624,820

U.S. Pat. No. 5,674,703

U.S. Pat. No. 5,677,196

U.S. Pat. No. 5,719,060

U.S. Pat. No. 5,766,920

U.S. Pat. No. 5,817,491

U.S. Pat. No. 5,869,463

U.S. Pat. No. 5,922,597

U.S. Pat. No. 5,928,944

U.S. Pat. No. 6,054,297

U.S. Pat. No. 6,383,805

U.S. Pat. No. 6,440,407

U.S. Pat. No. 6,475,725

International Application No. WO 86/01533

International Application No. WO 90/11354

International Application No. WO 90/02809

International Application No. WO 91/17271

International Application No. WO 91/01140

International Application No. WO 92/01047

International Application No. WO 92/09690

International Application No. WO 92/15679

International Application No. WO 92/18619

International Application No. WO 92/20791

International Application No. WO 93/01288

International Application No. WO 93/04169

International Application No. WO 96/26432

International Application No. WO 97/07668

International Application No. WO 97/07669

Aslam and Dent, “Bioconjugation: Protein Coupling Techniques for the Biomedical Sciences,” Macmillan Reference Ltd., London, England, 1998.

Baldari et al., Embo J. 6:229-234, 1987.

Banerji et al., Cell, 33:729-740; 1983.

Barany et al., Int. J. Peptide Protein Res., 30:705-739, 1987.

Barr et al., Gene Therapy 1:51-58,1994.

Berkner, BioTechniques 6:616-629,1998.

Berns and Bohenzky, Advances in Virus Research (Academic Press, Inc.) 32:243-307,1987.

Bett et al., J. Virol. 67:5911-5921,1993.

Blazer et al., “The Prevalence and Distribution of Major Depression in a National Community Sample, the National Comorbidity Survey,” Am J. Psychiatry, 151:979-986,1994.

Boris-Lawrie and Temin, Cur. Opin. Genet. Develop. 3:102-109,1993.

Bradley, Current Opinion in Biotechnology 2:823-829, 1991.

Bradley, in “Teratocarcinomas and Embryonic Stem Cells: A Practical Approach,” E. J. Robertson, ed., IRL, Oxford, pp. 113-152,1987.

Burns et al., Proc. Natl. Acad. Sci. USA 90:8033-8037,1993.

Byrne and Ruddle, PNAS 86:5473-5477,1989.

Calame and Eaton, Adv. Immunol. 43:235-275,1988.

Campes and Tilghman, Genes Dev. 3:537-546,1989.

Derisi et al., “Use of a cDNA microarray to analyse gene expression patterns in human cancer”, Nature Genetics, 14 (4): 457-460,1996.

Edlund et al., Science 230:912-916, 1985.

Fodor et al., “Light-directed, spatially addressable parallel chemical synthesis”, Science, 251 (4995):767-773,1991.

Flotte et al., Am. J. Respir. Cell Mol. Biol. 7:349-356,1992.

Flotte et al., J. Biol. Chem. 268:3781-3790,1993(a).

Flotte et al., Proc. Natl. Acad. Sci. USA 90:10613-10617,1993(b).

Frazer, “Pharmacology of antidepressants,” J. Clin. Psychopharmacol., 17, suppl. 1 2S-18S, 1997.

Fukata et al., “CRMP-2 binds to tubulin heterodimers to promote microtubule assembly,” Nat. Cell Biol., (8) 583-591, 2002.

Haj-Ahmad and Graham, J. Virol. 57:267-274,1986.

Högbom et al., “Structural basis for recognition by an in vitro evolved affibody,” Proc. Natl. Acad. Sci., USA, 100(6):3191-3196, 2003.

Johnson et al., Endoc. Rev., 10:317-331,1989.

Kaplitt et al., Nature Genetics 8:148-154,1994.

Kaufman et al., EMBO J. 6:187-195,1987.

Kempermann, “Regulation of adult hippocampal neurogenesis—implications for novel theories of major depression,” Bipolar Disord., 4(1):17-33, 2002.

Kessel and Gruss, Science 249:3 74-3 79, 1990.

Kessler et al., “Lifetime and 12-month prevalence of DSM-11′-R psychiatric disorders in the United States,” Arch. Gen. Psychiatry 51:8-19,1994.

Kim, Macromolecules 22:2682-2685, 1984.

Klerman et al., “Birth-cohort Trends in Rates of Major Depressive Disorder Among Relatives of Patients with Affective Disorder,” Arch. Gen. Psychiatry, 42:689-693, 1985.

Kurjan and Herskowitz, Cell 933-943, 1982.

Kyte and Doolittle, J. Mol. Biol., 157:105-132,1982.

Lakso et al., PJVAS 89:6232-6236, 1992.

Lipshutz et al., “Using oligonucleotide probe arrays to access genetic diversity”, BioTechniques, 19(3):442-447,1995.

Lissens et al., “Mutations in the X-linked pyruvate dehydrogenase (E1) alpha subunit gene (PDHA1) in patients with a pyruvate dehydrogenase complex deficiency,” Hum Mutat., 15(3):209-19, 2000.

Livache et al., “Preparation of a DNA matrix via an electrochemically directed copolymerization of pyrrole and oligonucleotides bearing a pyrrole group”, Nucleic Acids Research, 22(15):2915-2921,1994.

Lockhart et al., “Expression monitoring by hybridization to high-density oligonucleotide arrays”, Nature Biotechnology, 14(13):1675-1680, 1996.

Maher, Bioassays 14(12):807-15,1992.

Malberg et al., “Chronic antidepressant treatment increases neurogenesis in adult rat hippocampus,” Journal of Neuroscience, 20(24):9104-10, 2000.

Manji et al., “The cellular neurobiology of depression,” Nat Med., 7(5):541-7, 2001.

Miller and Rosman, BioTechniques 7:980-990,1989.

Miller, Human Gene Therapy 1:5-14, 1990.

Mittereder et al., Human Gene Therapy 5:717-729, 1994.

Murray and Lopez (eds.), “The Global Burden of Disease (Harvard University Press, Boston, 1996)

Nemeroff and Owens, “Treatment of Mood Disorders, Nature Neuroscience Supplement, 5: 1068-1070, 2002.

Nestler, et al., “Neurobiology of depression,” Neuron., 34(1):13-25, 2002.

Netto et al., “Thiol enzymes protecting mitochondria against oxidative damage,” Methods Enzymol., 348:260-70, 2002.

Nord et al., “Binding proteins selected from combinatorial libraries of an—helical bacterial receptor domain,” Nature Biotechnol. 15: 772-777,1997.

O'Gon-nan et al., Science 251:1351-1355,1991.

Ojika, et al., “Hippocampal cholinergic neurostimulating peptides (HCNP),” Prog Neurobiol., 60(1):37-83, 2000.

Okuda et al., “PCTAIRE-1 and PCTAIRE-3, two members of a novel cdc2/CDC28-related protein kinase gene family,” Oncogene, 7(11):2249-58,1992.

Paxinos, Watson and Emson, “AChE-stained horizontal sections of the rat brain in stereotaxic coordinates,” J. Neurosci. Methods, 3(2):129-149,1980.

Phelan, “AOP2 (antioxidant protein 2): structure and function of a unique thiol-specific antioxidant,” Antioxid Redox Signal, 1 (4):571-84,1999.

Pinkert et al. Genes Dev. 1:268-277,1987.

Queen and Baltimore, Cell 33:741-748,1983.

Rich et al., Human Gene Therapy 4:461-476, 1993.

Rönnmark et al., “Construction and characterization of affibody-Fc chimeras produced in Escherichia coli,” J. Immunol. Meth., 261:199-211, 2002.

Sambrook et al., “Molecular Cloning: A Laboratory Manual” 2nd, ed, Cold Spring Harbor Laboratory, Cold Spring Harbor Laboratory Press, Cold Spring Harbor, N.Y., 1989.

Scarpa et al., Virology 180:849-852,1991.

Schaaf et al., Langmuir 3:1131-1135,1987.

Schultz et al., Gene 54:113-123, 1987.

Seed, Nature 329:840, 1987.

Seth et al., J. Virol. 68:933-940, 1994.

Shirayama et al. “Brain-derived neurotrophic factor produces antidepressant effects in behavioral models of depression,” J. Neurosci., 22(8):3251-61, 2002.

Shoemaker et al., “Quantitative phenotypic analysis of yeast deletion mutants using a highly parallel molecular bar-coding strategy”, Nature Genetics, 14(4):450-456,1996.

Smith and Johnson, Gene 67:31-40,1988.

Steenbergh et al., “Complete nucleotide sequence of the high molecular weight human IGF-I mRNA,” Biochem. Biophys. Res. Commun., 175(2):507-514, 1991.

Studier et al. “Gene Expression Technology” Methods in Enzymology 185, 60-89, 1990.

Thomas and Capecchi, “Site-directed mutagenesis by gene targeting in mouse embryo-derived stem cells,” Cell, 51 (3):503-512, 1987.

Tran et al., “Chromosomal localization, gene structure, and expression pattern of DDAH1: comparison with DDAH2 and implications for evolutionary origins,” Genomics, 68(1):101-5, 2000.

Wang et al., “A family of rat CRMP genes is differentially expressed in the nervous system. J. Neurosci., 16(19):6197-207,1996.

Wang et al., “Polyclonal antibody localizes glia maturation factor beta-like immunoreactivity in neurons and glia,” Brain Research, 591(1):1-7 (1992).

Wilmut et al., Nature 385:810-813, 1997.

Winoto and Baltimore. EMBO J. 8:729-733,1989.

Wolff et al., “Grafting fibroblasts genetically modified to produce L-Dopa in a rat model of Parkinson's disease,” Proc. Natl. Acad. Sci. USA 86:9011-9014, 1987.

Yershov et al., “DNA analysis and diagnostics on oligonucleotide microchips”, Proceedings Of The National Academy Of Sciences Of The United States Of America, 93(10):4913-4918,1996.

Yeung et al., “Mechanism of suppression of the Raf/MEK/extracellular signal-regulated kinase pathway by the raf kinase inhibitor protein,” Mol Cell Biol., 20(9):3079-85, 2000.

Yildiz et al., “Antidepressant-like effect of 7-nitroindazole in the forced swimming test in rats,” Psychopharmacol., 149(1):41-4, 2000.

Zaheer et al., “Enhanced expression of neurotrophic factors by C6 rat glioma cells after transfection with glia maturation factor,” Neurosci Lett., 265(3):203-6, 1999.

Zaheer et al., “In vitro inhibition of MAP kinase (ERK1/ERK2) activity by phosphorylated glia maturation factor (GMF),” Biochemistry, 35:6283-6288 1996.

What is claimed is:

1. A method of screening for a mood disorder in a human subject comprising the steps of:
(a) obtaining a biological sample from the subject;
(b) contacting the sample with a polynucleotide probe complementary to an IGF-1 mRNA;
(c) measuring the amount of probe bound to the mRNA; and
(d) comparing the amount in step (c) with IGF-1 mRNA in human samples obtained from a statistically significant population lacking the mood disorder,
wherein lower IGF-1 mRNA levels in the subject indicates a predisposition to the mood disorder.

2. A method of screening for a mood disorder in a human subject comprising the steps of:
(a) obtaining a biological sample from the subject;
(b) contacting the sample with a polynucleotide probe complementary to a GMF-β mRNA;
(c) measuring the amount of probe bound to the mRNA;
(d) comparing the amount in step (c) with GMF-β mRNA in human samples obtained from a statistically significant population lacking the mood disorder,
wherein lower GMF-β mRNA levels in the subject indicates a predisposition to the mood disorder.

3. A method of screening for a mood disorder in a human subject comprising the steps of:
(a) obtaining a biological sample from the subject;
(b) contacting the sample with a polynucleotide probe complementary to a collapsin response mediator protein 2 (CRMP2) mRNA;
(c) measuring the amount of probe bound to the mRNA; and
(d) comparing the amount in step (c) with CRMP2 mRNA in human samples obtained from a statistically significant population lacking the mood disorder,
wherein lower CRMP2 mRNA levels in the subject indicates a predisposition to the mood disorder.

4. A method of screening for a mood disorder in a human subject comprising the steps of:
(a) obtaining a biological sample from the subject;
(b) contacting the sample with a polynucleotide probe complementary to a PCTAIRE-3 mRNA;
(c) measuring the amount of probe bound to the mRNA; and
(d) comparing the amount in step (c) with PCTAIRE-3 mRNA in human samples obtained from a statistically significant population lacking the mood disorder,
wherein lower PCTAIRE-3 mRNA levels in the subject indicates a predisposition to the mood disorder.

5. A method of screening for a mood disorder in a human subject comprising the steps of:
(a) obtaining a biological sample from the subject;
(b) contacting the sample with a polynucleotide probe complementary to a HCNP precursor protein mRNA;
(c) measuring the amount of probe bound to the mRNA; and
(d) comparing the amount in step (c) with HCNP precursor protein mRNA in human samples obtained from a statistically significant population lacking the mood disorder,
wherein lower HCNP precursor protein mRNA levels in the subject indicates a predisposition to the mood disorder.

6. A method of screening for a mood disorder in a human subject comprising the steps of:
(a) obtaining a biological sample from the subject;
(b) contacting the sample with a polynucleotide probe complementary to a hydroxysteroid sulfotransferase mRNA;
(c) measuring the amount of probe bound to the mRNA; and
(d) comparing the amount in step (c) with hydroxysteroid sulfotransferase mRNA in human samples obtained from a statistically significant population lacking the mood disorder,
wherein lower hydroxysteroid sulfotransferase mRNA levels in the subject indicates a predisposition to the mood disorder.

7. A method of screening for a mood disorder in a human subject comprising the steps of:
(a) obtaining a biological sample from the subject;
(b) contacting the sample with a polynucleotide probe complementary to a pyruvate dehydrogenase-E1 mRNA;
(c) measuring the amount of probe bound to the mRNA; and
(d) comparing the amount in step (c) with pyruvate dehydrogenase-E1 mRNA in human samples obtained from a statistically significant population lacking the mood disorder,
wherein lower pyruvate dehydrogenase-E1 mRNA levels in the subject indicates a predisposition to the mood disorder.

8. A method of screening for a mood disorder in a human subject comprising the steps of:
(a) obtaining a biological sample from the subject;
(b) contacting the sample with a polynucleotide probe complementary to an antioxidant protein-2 mRNA;
(c) measuring the amount of probe bound to the mRNA; and
(d) comparing the amount in step (c) with antioxidant protein-2 mRNA in human samples obtained from a statistically significant population lacking the mood disorder,
wherein lower antioxidant protein-2 mRNA levels in the subject indicates a predisposition to the mood disorder.

9. A method of screening for a mood disorder in a human subject comprising the steps of:
(a) obtaining a biological sample from the subject;
(b) contacting the sample with a polynucleotide probe complementary to a DDAH-1 mRNA;
(c) measuring the amount of probe bound to the mRNA; and
(d) comparing the amount in step (c) with DDAH-1 mRNA in human samples obtained from a statistically significant population lacking the mood disorder,
wherein lower DDAH-1 mRNA levels in the subject indicates a predisposition to the mood disorder.

10. The method according to any one of claims 1-9, wherein the mood disorder is selected from the group consisting of unipolar depressive disorder, bipolar depressive disorder, anxiety disorder, panic disorder, dysthymic disorder, postpartum depressive disorder, chronic major depressive disorder and double depressive disorder.

11. The method according to any one of claims 1-9, wherein the biological sample is obtained as a blood sample, a cerebrospinal fluid (CSF) sample, a saliva sample, a skin biopsy, a brain biopsy or a buccal biopsy.

12. The method according to any one of claims 1-9, wherein the biological sample is selected from the group consisting of blood plasma, serum, erythrocytes, leukocytes, platelets, lymphocytes, macrophages, fibroblast cells, mast cells, fat cells, epithelial cells, nerve cells, glial cells, Schwann cells and progenitor stem cells.

13. The method of claim 1, wherein the probe comprises a nucleotide sequence which hybridizes under high stringency hybridization conditions with a polynucleotide comprising the nucleotide sequence of SEQ ID NO:1.

14. The method of claim 2, wherein the probe comprises a nucleotide sequence which hybridizes under high stringency hybridization conditions with a polynucleotide comprising the nucleotide sequence of SEQ ID NO:3.

15. The method of claim 3, wherein the probe comprises a nucleotide sequence which hybridizes under high stringency hybridization conditions with a polynucleotide comprising the nucleotide sequence of SEQ ID NO:5.

16. The method of claim 4, wherein the probe comprises a nucleotide sequence which hybridizes under high stringency hybridization conditions with a polynucleotide comprising the nucleotide sequence of SEQ ID NO:7.

17. The method of claim 5, wherein the probe comprises a nucleotide sequence which hybridizes under high stringency hybridization conditions with a polynucleotide comprising the nucleotide sequence of SEQ ID NO:9.

18. The method of claim 6, wherein the probe comprises a nucleotide sequence which hybridizes under high stringency hybridization conditions with a polynucleotide comprising the nucleotide sequence of SEQ ID NO:11.

19. The method of claim 7, wherein the probe comprises a nucleotide sequence which hybridizes under high stringency hybridization conditions with a polynucleotide comprising the nucleotide sequence of SEQ ID NO:13.

20. The method of claim 8, wherein the probe comprises a nucleotide sequence which hybridizes under high stringency hybridization conditions with a polynucleotide comprising the nucleotide sequence of SEQ ID NO:15.

21. The method of claim 9, wherein the probe comprises a nucleotide sequence which hybridizes under high stringency hybridization conditions with a polynucleotide comprising the nucleotide sequence of SEQ ID NO:17.

22. A method of screening for a mood disorder in a human subject comprising:
(a) obtaining a biological sample from the subject;
(b) obtaining a biological control sample from a statistically significant human population lacking the mood disorder;
(c) contacting the samples in step (a) and step (b) with a plurality of polynucleotide probes, wherein the probes are complementary to an IGF-1 mRNA, a GMF-β mRNA, a CRMP2 mRNA, a PCTAIRE-3 mRNA, a HCNP mRNA, a hydroxysteroid sulfotransferase mRNA, a pyruvate dehydrogenase mRNA, an antioxidant protein-2 mRNA and a DDAH-1 mRNA;
(d) measuring the amount of each probe bound to the mRNA in step (c); and
(e) comparing the amount measured in step (d) from the subject sample relative to the amount measured in step (d) from the control sample,
wherein lower levels of one or more mRNAs measured in the subject sample relative to the control sample, indicates that the subject has a predisposition to the mood disorder.

23. A method of screening for a mood disorder in a human subject comprising:
(a) obtaining a biological sample from the subject;
(b) obtaining a biological control sample from a statistically significant human population lacking the mood disorder;
(c) contacting the samples in step (a) and step (b) with a plurality of antibodies, wherein the plurality of antibodies specifically bind an IGF-1 protein, a GMF-β protein, a CRMP2 protein, a PCTAIRE-3 protein, a HCNP protein, a hydroxysteroid sulfotransferase protein, a pyruvate dehydrogenase protein, an antioxidant protein-2 protein and a DDAH-1 protein;
(d) measuring the amount of each antibody bound to its respective protein in step (c);
(e) comparing the amount measured in step (d) from the subject sample relative to the amount measured in step (d) from the control sample,
wherein lower levels of one or more proteins measured in the subject sample relative to the control sample, indicates that the subject has a predisposition to the mood disorder.

24. A method for monitoring the kinetics of an inhibitor of a monoamine re-uptake receptor in a rodent comprising the steps of:
(a) administering to a plurality of rodents a monoamine re-uptake inhibitor or a placebo;
(b) obtaining, at a desired time point, a hippocampus from one of the plurality of rodents administered the monoamine re-uptake inhibitor in step (a) and a hippocampus from one of the plurality of rodents administered a placebo in step (a);
(c) determining the amount of one or more proteins in the hippocampus from step (b), wherein the one or more proteins are selected from the group consisting of IGF-1, GMF-β, CRMP2, PCTAIRE-3, HCNP, hydroxysteroid sulfotransferase, a pyruvate dehydrogenase, antioxidant protein-2 and DDAH-1,
(d) repeating steps (b) and (c), wherein a range of desired time points are gathered from 0 days to about 36 days.

25. A method for monitoring the kinetics of an inhibitor of a monoamine re-uptake receptor in a recombinant cell comprising the steps of:
(a) administering to a plurality of the recombinant cells a monoamine re-uptake inhibitor or a placebo;
(b) obtaining, at a desired time point, a cell from one of the plurality of cells administered the monoamine re-uptake inhibitor in step (a) and a cell from one of the plurality of cells administered a placebo in step (a);
(c) determining the amount of one or more proteins in the cell from step (b), wherein the one or more proteins are selected from the group consisting of IGF-1, GMF-β, CRMP2, PCTAIRE-3, HCNP, hydroxysteroid sulfotransferase, a pyruvate dehydrogenase, antioxidant protein-2 and DDAH-1,
(d) repeating steps (b) and (c), wherein a range of desired time points are gathered from 0 days to about 36 days.

26. A method of screening for an inhibitor of a monoamine re-uptake receptor comprising the steps of:
(a) contacting a mammalian cell with a test compound; and
(b) detecting the level of one or more proteins selected from the group consisting of IGF-1, GMF-β, CRMP2, PCTAIRE-3, HCNP, hydroxysteroid sulfotransferase, pyruvate dehydrogenase, antioxidant protein-2 and DDAH-1, wherein an increase in level of the one or more proteins, relative to the level of the one or more proteins in the absence of the test compound, indicates the test compound is an inhibitor of a monoamine re-uptake receptor.

27. A transgenic non-human animal comprising one or more exogenous polynucleotides encoding a protein selected from the group consisting of an IGF-1, a GMF-β, a CRMP2, a PCTAIRE-3, a HCNP, a hydroxysteroid sulfotransferase, a pyruvate dehydrogenase, an antioxidant protein-2 and a DDAH-1 protein.

28. A transgenic non-human animal having a functional disruption in one or more genes encoding a protein selected from the group consisting of an IGF-1, a GMF-β, a CRMP2, a PCTAIRE-3, a HCNP, a hydroxysteroid sulfotransferase, a pyruvate dehydrogenase, an antioxidant protein-2 and a DDAH-1 protein.

29. The animal of claim 28, wherein the animal is heterozygous for the one or more disruptions.

30. A method of screening for a mood disorder in a human subject comprising:
(a) obtaining a biological sample from the subject;
(b) applying the sample to a DNA chip comprising an array of polynucleotides, wherein the array comprises at least a nucleotide sequence encoding an IGF-1 protein, a GMF-β protein, a CRMP2 protein, a PCTAIRE-3 protein, a HCNP protein, a hydroxysteroid sulfotransferase protein, a pyruvate dehydrogenase protein, an antioxidant protein-2 protein and a DDAH-1 protein;
(c) measuring the amount of each polynucleotide bound to the array; and
(d) comparing the amount in step (c) with IGF-1, GMF-β, CRMP2, PCTAIRE-3, HCNP, hydroxysteroid sulfotransferase, pyruvate dehydrogenase, antioxidant protein-2 and DDAH-1 polynucleotide levels in human samples obtained from a statistically significant population lacking the mood disorder,
wherein lower levels of one or more polynucleotides in the subject indicates a predisposition to the mood disorder.

31. A method for screening for a mood disorder in a human subject comprising:
(a) obtaining a biological sample from the subject;
(b) applying the sample to an array of protein-capture agents, wherein a protein-capture agent on the array can bind an IGF-1 protein, a protein-capture agent on the array can bind GMF-β protein, a protein-capture agent on the array can bind a CRMP2 protein, a protein-capture agent on the array can bind a PCTAIRE-3 protein, a protein-capture agent on the array can bind a human HCNP protein, a protein-capture agent on the array can bind a human hydroxysteroid sulfotransferase protein, a protein-capture agent on the array can bind a human pyruvate dehydrogenase-E1 protein, a protein-capture agent on the array can bind a human antioxidant protein-2 protein and a protein-capture agent on the array can bind a human DDAH-1 protein;
(c) measuring the amount of each bound protein; and
(d) comparing the amount in step (c) with an array standard obtained from a statistically significant population lacking the mood disorder,
wherein lower levels of one or more proteins in the subject indicates a predisposition to the mood disorder.

32. The method of claim 31, wherein the protein-capture agent is an antibody.

33. A method for treating a mood disorder in a human subject in need thereof comprising delivering a polynucleotide encoding a wild-type IGF-1 polypeptide.

34. A method for treating a mood disorder in a human subject in need thereof comprising delivering a polynucleotide encoding a wild-type GMF-β polypeptide.

35. A method for treating a mood disorder in a human subject in need thereof comprising delivering a polynucleotide encoding a wild-type CRMP2 polypeptide.

36. A method for treating a mood disorder in a human subject in need thereof comprising delivering a polynucleotide encoding a wild-type PCTAIRE-3 polypeptide.

37. A method for treating a mood disorder in a human subject in need thereof comprising delivering a polynucleotide encoding a wild-type HCNP polypeptide.

38. A method for treating a mood disorder in a human subject in need thereof comprising delivering a polynucleotide encoding a wild-type hydroxysteroid sulfotransferase polypeptide.

39. A method for treating a mood disorder in a human subject in need thereof comprising delivering a polynucleotide encoding a wild-type pyruvate dehydrogenase polypeptide.

40. A method for treating a mood disorder in a human subject in need thereof comprising delivering a polynucleotide encoding a wild-type antioxidant protein-2 polypeptide.

41. A method for treating a mood disorder in a human subject in need thereof comprising delivering a polynucleotide encoding a wild-type DDAH-1 polypeptide.

42. A method for treating a mood disorder in a human subject in need thereof comprising delivering one or more polynucleotides encoding a wild-type IGF-1 polypeptide, a GMF-β polypeptide, a CRMP2 polypeptide, a PCTAIRE-3 polypeptide, a HCNP polypeptide, a hydroxysteroid sulfotransferase polypeptide, a pyruvate dehydrogenase polypeptide, an antioxidant protein-2 polypeptide and a DDAH-1 polypeptide.


SEQUENCE LISTING (18 sequences)

SEQ ID NO: 1 - DNA, 7260 bp, Homo sapiens
tcactgtcactgctaaattcagagcagattagagcctgcgcaatggaataaagtcctcaaaattgaaatgtgacattgctctcaacatctcccatctctctggatttccttttgcttcattattcctgctaaccaattcattttcagactttgtacttcagaagcaatgggaaaaatcagcagtcttccaacccaattatttaagtgctgcttttgtgatttcttgaaggtgaagatgcacaccatgtcctcctcgcatctcttctacctggcgctgtgcctgctcaccttcaccagctctgccacggctggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaaggaagtacatttgaagaacgcaagtagagggagtgcaggaaacaagaactacaggatgtaggaagaccctcctgaggagtgaagagtgacatgccaccgcaggatcctttgctctgcacgagttacctgttaaactttggaacacctaccaaaaaataagtttgataacatttaaaagatgggcgtttcccccaatgaaatacacaagtaaacattccaacattgtctttaggagtgatttgcaccttgcaaaaatggtcctggagttggtagattgctgttgatcttttatcaataatgttctatagaaaagaaaaaaaaatatatatatatatatatcttagtccctgcctctcaagagccacaaatgcatgggtgttgtatagatccagttgcactaaattcctctctgaatcttggctgctggagccattcattcagcaaccttgtctaagtggtttatgaattgtttccttatttgcacttctttctacacaactcgggctgtttgttttacagtgtctgataatcttgttagtctatacccaccacctcccttcataacctttatatttgccgaatttggcctcctcaaaagcagcagcaagtcgtcaagaagcacaccaattctaacccacaagattccatctgtggcatttgtaccaaatataagttggatgcattttattttagacacaaagctttatttttccacatcatgcttacaaaaaagaataatgcaaatagttgcaactttgaggccaatcatttttaggcatatgttttaaacatagaaagtttcttcaactcaaaagagttccttcaaatgatgagttaatgtgcaacctaattagtaactttcctctttttattttttccatatagagcactatgtaaatttagcatatcaattatacaggatatatcaaacagtatgtaaaactctgttttttagtataatggtgctattttgtagtttgttatatgaaagagtctggccaaaacggtaatacgtgaaagcaaaacaataggggaagcctggagccaaagatgacacaaggggaagggtactgaaaacaccatccatttgggaaagaaggcaaagtccccccagttatgccttccaagaggaacttcagacacaaaagtccactgatgcaaattggactggcgagtccagagaggaaactgtggaatggaaaaagcagaaggctaggaattttagcagtcctggtttctttttctcatggaagaaatgaacatctgccagctgtgtcatggactcaccactgtgtgaccttgggcaagtcacttcacctctctgtgcctcagtttcctcatctgcaaaatgggggcaatatgtcatctacctacctcaaaggggtggtataaggtttaaaaagataaagattcagatttttttaccctgggttgctgtaagggtgcaacatcagggcgcttgagttgctgagatgcaaggaattctataaataacccattcatagcatagctagagattggtgaattgaatgctcctgacatctcagttcttgtcagtgaagctatccaaataactggccaactagttgttaaaagctaacagctcaatctcttaaaacacttttcaaaatatgtgggaagcatttgattttcaatttgattttgaattctgcatttggttttatgaatacaaagataagtgaaaagagagaaaggaaaagaaaaaggagaaaaacaaagagatttctaccagtgaaaggggaattaattactctttgttagcactcactgactcttctatgcagttactacatatctagtaaaaccttgtttaatactataaataatattctattcattttgaaaaacacaatgattccttcttttctaggcaatataaggaaagtgatccaaaatttgaaatattaaaataatatctaataaaaagtcacaaagttatcttctttaacaaactttactcttattcttagctgtatatacatttttttaaaaagtttgttaaaatatgcttgactagagtttcagttgaaaggcaaaaacttccatcacaacaagaaatttcccatgcctgctcagaagggtagcccctagctctctgtgaatgtgttttatccattcaactgaaaattggtatcaagaaagtccactggttagtgtactagtccatcatagcctagaaaatgatccctatctgcagatcaagattttctcattagaacaatgaattatccagcattcagatctttctagtcaccttagaactttttggttaaaagtacccaggcttgattatttcatgcaaattctatattttacattcttggaaagtctatatgaaaaacaaaaataacatcttcagtttttctcccactgggtcacctcaaggatcagaggccaggaaaaaaaaaaaagactccctggatctctgaatatatgcaaaaagaaggccccatttagtggagccagcaatcctgttcagtcaacaagtattttaactctcagtccaacattatttgaattgagcacctcaagcatgcttagcaatgttctaatcactatggacagatgtaaaagaaactatacatcatttttgccctctgcctgttttccagacatacaggttctgtggaataagatactggactcctcttcccaagatggcacttctttttatttcttgtccccagtgtgtaccttttaaaattattccctctcaacaaaactttataggcagtcttctgcagacttaacatgttttctgtcatagttagatgtgataattctaagagtgtctatgacttatttccttcacttaattctatccacagtcaaaaatcccccaaggaggaaagctgaaagatgcaactgccaatattatctttcttaactttttccaacacataatcctctccaactggattataaataaattgaaaataactcattataccaattcactattttattttttaatgaattaaaactagaaaacaaattgatgcaaaccctggaagtcagttgattactatatactacagcagaatgactcagatttcatagaaaggagcaaccaaaatgtcacaaccaaaactttacaagctttgcttcagaattagattgctttataattcttgaatgaggcaatttcaagatatttgtaaaagaacagtaaacattggtaagaatgagctttcaactcataggcttatttccaatttaattgaccatactggatacttaggtcaaatttctgttctctcttgcccaaataatattaaagtattatttgaactttttaagatgaggcagttcccctgaaaaagttaatgcagctctccatcagaatccactcttctagggatatgaaaatctcttaacacccaccctacatacacagacacacacacacacacacacacacacacacacacacacacattcaccctaaggatccaatggaatactgaaaagaaatcacttccttgaaaattttattaaaaaacaaacaaacaaacaaaaagcctgtccacccttgagaatccttcctctccttggaacgtcaatgtttgtgtagatgaaaccatctcatgctctgtggctccagggtttctgttactattttatgcacttgggagaaggcttagaataaaagatgtagcacattttgctttcccatttattgtttggccagctatgccaatgtggtgctattgtttctttaagaaagtacttgactaaaaaaaaaagaaaaaaagaaaaaaaagaaagcatagacatatttttttaaagtataaaaacaacaattctatagatagatggcttaataaaatagcattaggtctatctagccaccaccacctttcaactttttatcactcacaagtagtgtactgttcaccaaattgtgaatttgggggtgcaggggcaggagttggaaattttttaaagttagaaggctccattgttttgttggctctcaaacttagcaaaattagcaatatattatccaatcttctgaacttgatcaagagcatggagaataaacgcgggaaaaaagatcttataggcaaatagaagaatttaaaagataagtaagttccttattgatttttgtgcactctgctctaaaacagatattcagcaagtggagaaaataagaacaaagagaaaaaatacatagatttacctgcaaaaaatagcttctgccaaatcccccttgggtattctttggcatttactggtttatagaagacattctcccttcacccagacatctcaaagagcagtagctctcatgaaaagcaatcactgatctcatttgggaaatgttggaaagtatttccttatgagatgggggttatctactgataaagaaagaatttatgagaaattgttgaaagagatggctaacaatctgtgaagattttttgtttcttggttttgtttttttttttttttttactttatacagtctttatgaatttcttaatgttcaaaatgacttggttcttttcttcttttttttatatcagaatgaggaataataagttaaacccacatagactctttaaaactataggctagatagaaatgtatgtttgacttgttgaagctataatcagactatttaaaatgttttgctatttttaatcttaaaagattgtgctaatttattagagcagaacctgtttggctctcctcagaagaaagaatctttccattcaaatcacatggctttccaccaatattttcaaaagataaatctgatttatgcaatggcatcatttattttaaaacagaagaattgtgaaagtttatgcccctcccttgcaaagaccataaagtccagatctggtaggggggcaacaacaaaaggaaaatgttgttgattcttggttttggattttgttttgttttcaatgctagtgtttaatcctgtagtacatatttgcttattgctattttaatattttataagaccttcctgttaggtattagaaagtgatacatagatatcttttttgtgtaatttctatttaaaaaagagagaagactgtcagaagctttaagtgcatatggtacaggataaagatatcaatttaaataaccaattcctatctggaacaatgcttttgttttttaaagaaacctctcacagataagacagaggcccaggggatttttgaagctgtctttattctgcccccatcccaacccagcccttattattttagtatctgcctcagaattttatagagggctgaccaagctgaaactctagaattaaaggaacctcactgaaaacatatatttcacgtgttccctctcttttttttcctttttgtgagatggggtctcgcactgtcccccaggctggagtgcagtggcatgatctcggctcactgcaacctccacctcctgggtttaagcgattctcctgcctcagcctcctgagtagctgggattacaggcacccaccactatgcccggctaattttttggatttttaatagagacggggttttaccatgttggccaggttggactcaaactcctgaccttgtgatttgcccgcctcagcctcccaaattgctgggattacaggcatgagccaccacaccctgcccatgtgttccctcttaatgtatgattacatggatcttaaacatgatccttctctcctcattcttcaactatctttgatggggtctttcaaggggaaaaaaatccaagcttttttaaagtaaaaaaaaaaaaagagaggacacaaaaccaaatgttactgctcaactgaaatatgagttaagatggagacagagtttctcctaataaccggagctgaattacctttcactttcaaaaacatgaccttccacaatccttagaatctgcctttttttatattactgaggcctaaaagtaaacattactcattttattttgcccaaaatgcactgatgtaaagtaggaaaaataaaaacagagctctaaaatccctttcaagccacccattgaccccactcaccaactcatagcaaagtcacttctgttaatcccttaatctgattttgtttggatatttatcttgtacccgctgctaaacacactgcaggagggactctgaaacctcaagctgtctacttacatcttttatctgtgtctgtgtatcatgaaaatgtctattcaaaatatcaaaacctttcaaatatcacgcagcttatattcagtttacataaaggccccaaataccatgtcagatctttttggtaaaagagttaatgaactatgagaattgggattacatcatgtattttgcctcatgtatttttatcacacttataggccaagtgtgataaataaacttacagacactgaattaatttcccctgctactttgaaaccagaaaataatgactggccattcgttacatctgtcttagttgaaaagcatattttttattaaattaattctgattgtatttgaaattattattcaattcacttatggcagaggaatatcaatcctaatgacttctaaaaatgtaactaattgaatcattatcttacatttactgtttaataagcatattttgaaaatgtatggctagagtgtcataataaaatggtatatctttctttagtaattacaaaaaaaaaaaaaaaaaaaaaaaa

SEQ ID NO: 2 - protein, 153 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 3 - DNA, 4131 bp, Homo sapiens
ctgggccaggcgccggggcaggaagggaggcggccgccgtggccattcttaaaggcgcccgagtgtaggcgacaggccgctgacggcgggaaggaaaatgagtgagtctttggttgtttgtgatgttgccgaagatttagtggaaaagctgagaaagtttcgttttcgcaaagaaacgaacaacgctgctattataatgaagattgacaaggataaacgcctggtggtactggatgaggagcttgagggcatttcaccagatgaacttaaagatgaactacctgaacgacaacctcgcttcattgtgtatagttataaatatcaacatgatgatggaagagtttcatatcctctgtgctttattttctccagtcctgttggatgtaagcctgaacaacagatgatgtatgctggaagtaagaataagctagtccagacagctgaactaaccaaggtatttgaaataagaaataccgaagacctaactgaagaatggttacgtgagaaacttggattttttcactaatgtgaacttctgtgtttctaaagtatttatgtattaacctgaccatactggaatcagacataaatacttatttatgcctaaaaatgcactgttacttacagtttgtttcctgcagtaaagaaaaattcttcatttgtgcaaaatttgaacaaagaggaaatcatcttcatagtaatgaaactttgtaaagtgtttccttatattggtaattgttaggtggactacttttctccagggactttttgcactcttgtgactaatttctataacttatggttcggaatttgttactatttacagacaccattggaaagtggatatattagattgtgagagacaacagttgcctccttttgacaaatactggatattagcagtttatttatgaaaatagcgtattatcacttgtcaaatcattgaaattcatttggggtcaaagacttgagtgacccagtattgagccatgaataatttagtgtaacctgtattacaagtacattgatgaattctgtatcttctttggtttcctgtatctttttaatcaagtctagaaactatgttcatcagtcactcatttttaaggtcgggagttagattttatgatagaattatgactgttagcttttctccttatagcatcttagtcttagaaattggtgggttgtaataatcaagggcttcattccttttatgtcatttctagacagttttgaatctaggttaataacactttatttataaagcacctcaatgtcctgtgaacactaattattttaaatgtgttaatactgtgcctttgatttgttagctttaaagttagtttaagacttttacactgccagtattccacatttggtgaaattaatacttttttaaagggtccaaataaaataattttctaatgtgtatatctgaaatttgtaataaaatcaacttcatattttaaaaattccaactatctgcttgcattggtgtaatatatggcagtcgagagttataattttgggtatacttgtggttagttttgtgccataggaaaaaattatcttaaaactttggccatagttaataacattaacacttcaatagcaatcacatcttatatcctaaatgtcagaagatattctgaactggatgcctgaatagttaactaaaccagtcttggttagatgatggtactcttggcataaagcgtaggattctgatatttggcatacttgtaaaaacaaatacataagtaaccattgaacattaatttgataataggtctagagactctaaaaactaaccaaacttggtgagtgtattcttatattaagaatatcttagtcatctcaaaactagcaaaatttaaattttggcatgttttccattcatatgttctttgcattttatttttgaggtttctgtgagaagtaaagatagttggaatttttgcgatattgaatagaacatcttctgttcccaacactgtttggcttcactaatttagaagtcaggaagcaatagaaagttggagatgaggaagtgctagagtaggtgtttgttttggttcttggagggaaaagattctttattccaatttccagagagaagagaaaactcacccaggaagtttaaaaattctttaaacaggtattttgatattggagaataacatgcatataattctgtaggaatgcacatgtaatccaagtgagtggagagtgtttttaatgtttttgaatgaaggaaatgaggttttgtttcacctgttttgcagcagtaagagaaactagtgctgcaagaatgtattttttaatgaagttccttattttgtcttgcatgttttagttttgcttatttttaaatttggaggtcctccataatgtcagataatattgacctgccatacgttagcactcttagttccgctactgtctttaacaggagcaaagagctgtgataaaccatgcttttttgagcttgtctgactcctaattaataacatgtttttggcaagacaacagattgaggttagaggatcagtaggacatttttattccatctgtcctatggggaaatttacaaatcccgtgctctaaaatgttctcaaacatttatatagatttccctttcatcttactaaattttgcattgttcttttcaagtatgtttcgtatttactgtctttttttctgccatttcccaaataataactccagatttcataattccagtttttacattccgttatctttctggtacaaccattcccattcagccttaaatctgagtcctttttagcagcaacttttttcctgggatcctccttcgtggtcttctaagtcagtgttagttttgaaatttttggccctgcataagttctgcatagcatctaatgtcaaaatagaaccaactggtaatcacagtattatttagtgtggtttccatgacaacaaaaatacatacgaagaaaacttctcaggttactatgctgaaattccaaaatgtctgagttttgaatagtgatcactttgttctggtattgacgcaattatattaggaaaaaagttggttgactgtttttgtttaattgacttctaaaatgttcaaattgtctagttctaaaagtttactaaatgcctagtgcagttaaacatactcttgtttaagtgtgtgttgctaaattttttactgtcattactaaataatctgtgtggcaaaatgtgtgtcagcacttttccctccttttttatctcctattttcaggagtcaaatgtagccataaactgtatccttgtctgacactttagctaaaaatttccagttaggggagtttattgccaaattaaatttggctgttccccccaacccatatagatattaaggaaggtgtacttaaaaaatgtttggactgcttttaaaacctgagcaatgtcattaatccatatgtggactagtgatgaatagatattttcataagagtttaaatgactgatatttggtggaagtagagagtaactcaatattctatcaattcaagtattcttactatggttgcctttccctatttgttcaatagactgataatactggtatttatagagtttgagccattacaacttttgtgaggatgtgtttcaaacatttctggacaaatcttattttgtatttctggaagaatgtagtaatcttctagaccgcttaaaaccaatgctcccaagctgaatattcttgagcaaatttgttttttattatgccatttgacatttcaaatcagtgctcatatacagtaaacttgtgatagaaattgtattttattgctttttggattataattcatataaatataattacttgaatattgtttgagatcattaacatgccagggcagttcccactgatttagatggtccaagataatctcattcaggaggcttgaaacattaatggtttagtcttgtgaattttaacagttctctgtcatcgtttaacaaaaccaacaactgacacaactccttaagctgtggtttcagtctctgctagttcatattgcatgtttattttggacagtcttttgttaagcatggtgcttgtactggtttaaataaaatgttaacatt

SEQ ID NO: 4 - protein, 142 aa, Homo sapiens
MSESLVVCDVAEDLVEKLRKFRFRKETNNAAIIMKIDKDKRLVVLDEELEGISPDELKDELPERQPRFIVYSYKYQHDDGRVSYPLCFIFSSPVGCKPEQQMMYAGSKNKLVQTAELTKVFEIRNTEDLTEEWLREKLGFFH

SEQ ID NO: 5 - DNA, 4459 bp, Homo sapiens
gtttctctctctccttctctctctctctctctctctcttttttttccgccctagctggggctgtgttggaggagaggaagaaagagagacagaggattgcattcatccgttacgttcttgaaatttcctaatagcaagaccagcgaagcggttgcacccttttcaatcttgcaaaggaaaaaaacaaaacaaaacaaaaaaaacccaagtccccttcccggcagtttttgccttaaagctgccctcttgaaattaattttttcccaggagagagatgtcttatcaggggaagaaaaatattccacgcatcacgagcgatcgtcttctgatcaaaggaggtaaaattgttaatgatgaccagtcgttctatgcagacatatacatggaagatgggttgatcaagcaaataggagaaaatctgattgtgccaggaggagtgaagaccatcgaggcccactcccggatggtgatccccggaggaattgacgtccacactcgtttccagatgcctgatcagggaatgacgtctgctgatgatttcttccaaggaaccaaggcggccctggctgggggaaccactatgatcattgaccacgttgttcctgagcctgggacaagcctgctcgctgcctttgaccagtggagggaatgggccgacagcaagtcctgctgtgactactctctgcatgtggacatcagcgagtggcataagggcatccaggaggagatggaagcgcttgtgaaggatcacggggtaaattccttcctcgtgtacatggctttcaaagatcgcttccagctaacggattgccagatttatgaagtactgagtgtgatccgggatattggcgccatagcccaagtccacgcagaaaatggcgacatcattgcagaggagcagcagaggatcctggatctgggcatcacgggccccgagggacatgtgctgagccgacctgaggaggtcgaggccgaagccgtgaatcgtgccatcaccatcgccaaccagaccaactgcccgctgtatatcaccaaggtgatgagcaaaagctctgctgaggtcatcgcccaggcacggaagaagggaactgtggtgtatggcgagcccatcactgccagcttgggaacggacggctcccattactggagcaagaactgggccaaggctgctgcctttgtcacctccccacccttgagccctgatccaaccactccagactttctcaactccttgctgtcctgtggagacctccaggtcacgggcagtgcccattgcacgtttaacactgcccagaaggctgtaggaaaggacaacttcaccctgattccggagggcaccaatggcactgaggagcggatgtccgtcatctgggacaaggctgtggtcactgggaagatggatgagaaccagtttgtggctgtgaccagcaccaatgcagccaaagtcttcaacctttacccccggaaaggccgcattgctgtgggatccgatgccgacctggtcatctgggaccccgacagcgttaaaaccatctctgccaagacacacaacagctctctcgagtacaacatctttgaaggcatggagtgccgcggctccccactggtggtcatcagccaggggaagattgtcctggaggacggcaccctgcatgtcaccgaaggctctggacgctacattccccggaagcccttccctgattttgtttacaagcgtatcaaggcaaggagcaggctggctgagctgagaggggttcctcgtggcctgtatgacggacctgtgtgtgaagtgtctgtgacgcccaagacagtcactccagcctcctcggccaagacgtctcctgccaagcagcaggccccacctgtccggaacctgcaccagtctggattcagtttgtctggtgctcagattgatgacaacattccccgccgcaccacccagcgtatcgtggcgccccccggtggccgtgccaacatcaccagcctgggctagagctcctgggctgtgcgtccactggggactggggatgggacacctgaggacattctgagacttctttcttccttcctttttttttttttgtttttttttttaagagcctgtgatagttactgtggagcagccagttcatggggtcccccttggggccccacaccccgtctctcaccaagagttactgattttgctcatccacttccctacacatctatgggtatcacacccaagactacccaccaagctcatacagggaaccacacccaacacttagacatgcgaacaagcagcccccagcgagggtctccttcgccttcaacctcctagtgtctgttagcatcttccttttcatggggggagggaagataaagtgaattgcccagagctgcctttttcttttctttttaaaaattttaagaagttttccttgtggggctggggaggggccggggtcagggagagtctttttttttttttttttaaatactaaattggaacatttaattccatattaatacaaggggtttgaactggacatcctaatgatgcaattacgtcatcacccagctgattccgggtggttggcaaactcatcgtgtctgtcctgagaggctccacaatgcccacccgcatcgccattctgtagtcttcagggtcagctgttgataaaggggcaggcttgcgttattggcctagattttgctgcagattaaatcctttgaggattctcttctcttttaccatttttctgcgtgctctcactctctctttctctctctagctttttaattcatgaatattttcgtgtctgtctctctctctctctgtgtttcctccagcccttgtctcggagacggtgttttcctcccttgccccattatcttttcacctcccaggtctaccatttcatggtggtcgttgggtccgcctaaaggatttgagcgtttgccattgcaagcatagtgctgtgtcatcctggtccatgtaggactggtgctaaccacctgccatcatgaggatgtgtgctagagtgtgggaccctggccaagtgcaggaatgggccatgccgtctcacccacagtatcacacgtggaaccgcagacagggcccagaagctttagaggtatgaggctgcagaaccggagagattttcctctgtgcagtgctctctggctaaagtcacggtcaaacctaaacaccgagcctcattaacccaagtgaaccaaccaaagtcaccagttcagaagtgctaagctaataggagtctgacccgagggcctgctgcttcctggttaagtatcttttgagattctagaacacatgggagctttttattttcggggaaaaaccgtatttttttcttgtccaattatttctaaagacacactacatagaaagaggccctataaactcaaaaagtcattgggaaacttaaagtctattctactttgcaagaggagaaatgtgttttatgaacgatagatcacatcagaactcctgtggggaggaaaccttataaattaaacacatggcccccttagagaccacaggtgatgtctgtctccatccttccctctccttttctgtcacctttccccctagctggctcctttggacctacccctgtccttgctgacttgtgttgcattgtattccaaacgtgtttacaggttctcttaagcaatgttgtatttgcaggcttttctgaataccaaatctgctttttgtaaagcgtaaaaacatcacaaagtaggtcattccatcaccacccttgtctctctacacattttgcctttggggatctggttggggttttgggttttttgttgttgttgtttatttgttattttaaaggtaaattgcacttttaaaaaaataattggttgacttaatatatttgctttttttctcacctgcacttagaggaaatttgaacaagttggaaaaaaacaatttttgtttcaattctaagaaacacttgcagctctagtattcacttgagtcttcctgtttttcctgtaccgggtcatggtaatttttggttgttttggttgttttcttaaaaaacaagttaaaacctgacgatttctgcaggctgtgtaagcatgtttacctgttggcttgctttgtgtgtctgttaaatgaatgtcatatgtaaatgctaaaataaatcgacagtgtctcagaactgaataactgcagtgacttgatgctctaaaacagtgtaggatttaagaatagatggtttttaatcctggaaattgtgattgtgacccatgagtggaggaactttcagttctaaagctgataaagtgtgtagccagaagagtacttttttttttgtaaccactgtcttgatggcaaaataattatggtaaaaaacaagtctcgtgtttattattccttaagaactctgtgttatattaccatggaacgcctaataaagcaaaatgtggttgtttc

SEQ ID NO: 6 - protein, 572 aa, Homo sapiens
MSYQGKKNIPRITSDRLLIKGGKIVNDDQSFYADIYMEDGLIKQIGENLIVPGGVKTIEAHSRMVIPGGIDVHTRFQMPDQGMTSADDFFQGTKAALAGGTTMIIDHVVPEPGTSLLAAFDQWREWADSKSCCDYSLHVDISEWHKGIQEEMEALVKDHGVNSFLVYMAFKDRFQLTDCQIYEVLSVIRDIGAIAQVHAENGDIIAEEQQRILDLGITGPEGHVLSRPEEVEAEAVNRAITIANQTNCPLYITKVMSKSSAEVIAQARKKGTVVYGEPITASLGTDGSHYWSKNWAKAAAFVTSPPLSPDPTTPDFLNSLLSCGDLQVTGSAHCTFNTAQKAVGKDNFTLIPEGTNGTEERMSVIWDKAVVTGKMDENQFVAVTSTNAAKVFNLYPRKGRIAVGSDADLVIWDPDSVKTISAKTHNSSLEYNIFEGMECRGSPLVVISQGKIVLEDGTLHVTEGSGRYIPRKPFPDFVYKRIKARSRLAELRGVPRGLYDGPVCEVSVTPKTVTPASSAKTSPAKQQAPPVRNLHQSGFSLSGAQIDDNIPRRTTQRIVAPPGGRANITSLG

SEQ ID NO: 7 - DNA, 3094 bp, Homo sapiens
ccaccttcctccactccctcccttcccctcccttctacctcctcttcgccagctcaggttgcagcttctctggggaactgctcacctttccggagcaggggaagctgccccgtgcccgggagggagcgggcgcaccgcggcccccaggacacgcgctgtgagtcccgcgggcggtgcgcctgggaggaagggggaggtcggaggagcgggcaccgcggcgccgggtataaggagcaaaggacccggctgcccagtccctcatgatcatgaacaagatgaagaactttaagcgccgtttctccctgtcagtgccccgcactgagaccattgaagaatccttggctgaattcacggagcaattcaaccagctccacaaccggcggaatgagaacttgcagctcggtcctcttggcagagaccccccgcaggagtgcagcaccttctccccaacagacagcggggaggagccggggcagctctcccctggcgtgcagttccagcggcggcagaaccagcgccgcttctccatggaggacgtcagcaagaggctctctctgcccatggatatccgcctgccccaggaattcctacagaagctacagatggagagcccagatctgcccaagccgctcagccgcatgtcccgccgggcctccctgtcagacattggctttgggaaactggaaacatacgtgaaactggacaaactgggagagggcacctatgccacagtcttcaaagggcgcagcaaactgacggagaaccttgtggccctgaaagagatccggctggagcacgaggagggagcgccctgcactgccatccgagaggtgtctctgctgaagaacctgaagcacgccaatattgtgaccctgcatgacctcatccacacagatcggtccctcaccctggtgtttgagtacctggacagtgacctgaagcagtatctggaccactgtgggaacctcatgagcatgcacaacgtcaagattttcatgttccagctgctccggggcctcgcctactgtcaccaccgcaagatcctgcaccgggacctgaagccccagaacctgctcatcaacgagaggggggagctgaagctggccgactttggactggccagggccaagtcagtgcccacaaagacttactccaatgaggtggtgaccctgtggtacaggccccccgatgtgctgctgggatccacagagtactccacccccattgatatgtggggcgtgggctgcatccactacgagatggccacagggaggcccctcttcccgggctccacagtcaaggaggagctgcacctcatctttcgcctcctcgggacccccacagaagagacgtggcccggcgtgaccgccttctctgagttccgcacctacagcttcccctgctacctcccgcagccgctcatcaaccacgcgcccaggttggatacggatggcatccacctcctgagcagcctgctcctgtatgaatccaagagtcgcatgtcagcagaggctgccctgagtcactcctacttccggtctctgggagagcgtgtgcaccagcttgaagacactgcctccatcttctccctgaaggagatccagctccagaaggacccaggctaccgaggcttggccttccagcagccaggacgagggaagaacaggcggcagagcatcttctgagccacgcccaccttgctgtggccaagggacaagagatcacatggagcacaaattcgggtaggatggagcctgtgtggccctcggaggactgaagaacgagggctgacagccagcctggaagaccgcttggcagcccttctggccacggctgtttcttctttgtgcttcccgtgtgcctccccagtagccctcacctgcataccaacccctcctttacccacgttggggctggcataagctgcttccctgagaggacatgaggggggggcggtcctcgtaccctctcccaccctggtgtttgggcacctgcgtgggatgcacacggatgacagaatcaaggcgccaggatgggcactctgccctggatacaggctctaccctcctcccccaggacctgcctagtgccagtttggtagtccccctttctggccccttggagcccacacacgtttcatctttttcccctctgagagcaagaagagacatggcatgttctctgggaccctggaatcctaggtacccacatgtgtgccaaagcctaccccacctggcaggtgtcccacagcaacagaaggaatagtagtccccactctttccatcagccctaccctaccctcattccccgacaccctctggcttgaaccatggctgagcagtgccggcatacgctttgctggcatgcttggatgcccagctgtgtccagaggtggcctgggaccgccagttgcacgcctgccacctcagccagcccccgcccagctcatcagtctgaatggagttgccttaaattggcaggtggtaccgtactcactgcccttggagctgtgaccggctcctgcctgtccaccccttcccgaggtggctcctgcttaccttatcatcccagggctctgattagccaggcctggtcagggtcctggggacggcacccagatatgcagagtcaccctgacactggtgccaggctgacctcagctcccgaaggctcgcacagcctccccatccttccttcccagcccttgtggctctgtccacctgatcccaataccagcttcccccagcccctgccaccccagagggcggccacgacagggagaggtgtagatgccaccatctgagggagaggaacgtggaacaggagcaggctctgatgctgagaggcttgcctccgggggctggaagcctgggtggccggggcccctgaagaaggctcccctctgtatcccccaggtctcctcaacactgggctgatcctgaatggcacaggccaaggggaggccagcctcgcctttctacccaggccccctgccctgcccacctcaggcccccaccctccactcctccccacggtactgtgaacgtcgtgtgactcagtgcagagacagataatatatttaattcatgtacag

SEQ ID NO: 8 - protein, 472 aa, Homo sapiens [sequence omitted]

SEQ ID NO: 9 - DNA, 1434 bp, Homo sapiens
gagccagtgtgctgagctctccgcgtcgcctctgtcgcccgcgcctggcctaccgcggcactcccggctgcacgctctgcttggcctcgccatgccggtggacctcagcaagtggtccgggcccttgagcctgcaagaagtggacgagcagccgcagcacccgctgcatgtcacctacgccggggcggcggtggacgagctgggcaaagtgctgacgcccacccaggttaagaatagacccaccagcatttcgtgggatggtcttgattcagggaagctctacaccttggtcctgacagacccggatgctcccagcaggaaggatcccaaatacagagaatggcatcatttcctggtggtcaacatgaagggcaatgacatcagcagtggcacagtcctctccgattatgtgggctcggggcctcccaagggcacaggcctccaccgctatgtctggctggtttacgagcaggacaggccgctaaagtgtgacgagcccatcctcagcaaccgatctggagaccaccgtggcaaattcaaggtggcgtccttccgtaaaaagtatgagctcagggccccggtggctggcacgtgttaccaggccgagtgggatgactatgtgcccaaactgtacgagcagctgtctgggaagtagggggttagcttggggacctgaactgtcctggaggccccaagccatgttccccagttcagtgttgcatgtataatagatttctcctcttcctgccccccttggcatgggtgagacctgaccagtcagatggtagttgagggtgacttttcctgctgcctggcctttataattttactcactcactctgatttatgttttgatcaaatttgaacttcattttggggggtattttggtactgtgatggggtcatcaaattattaatctgaaaatagcaacccagaatgtaaaaaagaaaaagctggggggaaaaagaccaggtctacagtgatagagcaaagcatcaaagaatctttaagggaggtttaaaaaaaaaaaaaaaaaaaaagattggttgcctctgcctttgtgatcctgagtccagaatggtacacaatgtgattttatggtgatgtcactcacctagacaaccagaggctggcattgaggctaacctccaacacagtgcatctcagatgcctcagtaggcatcagtatgtcactctggtccctttaaagagcaatcctggaagaagcaggagggagggtggctttgctgttgttgggacatggcaatctagaccggcagcagcgctcgctgacagcttgggaggaaacctgagatctgtgttttttaaattgatcgttcttcatgggggtaagaaaagctggtctggagttgctgaatgttgcattaattgtgctgtttgcttgtagttgaataaaaatagaaacctgaatg

SEQ ID NO: 10 - protein, 187 aa, Homo sapiens
MPVDLSKWSGPLSLQEVDEQPQHPLHVTYAGAAVDELGKVLTPTQVKNRPTSISWDGLDSGKLYTLVLTDPDAPSRKDPKYREWHHFLVVNMKGNDISSGTVLSDYVGSGPPKGTGLHRYVWLVYEQDRPLKCDEPILSNRSGDHRGKFKVASFRKKYELRAPVAGTCYQAEWDDYVPKLYEQLSGK

SEQ ID NO: 11 - DNA, 1819 bp, Homo sapiens
gggacaaggttaaagatcgttttatccttgctgtaaaagctgatctgcctgtagctgccacagcctccagcggtggctacagttgaaaccctcacaccacgcaggaagaggtcatcatcatgtcggacgatttcttatggtttgaaggcatagctttccctactatgggtttcagatccgaaaccttaagaaaagtacgtgatgagttcgtgataagggatgaagatgtaataatattgacttaccccaaatcaggaacaaactggttggctgagattctctgcctgatgcactccaagggggatgccaagtggatccaatctgtgcccatctgggagcgatcaccctgggtagagagtgagattgggtatacagcactcagtgaaacggagagtccacgtttattctcctcccacctccccatccagttattccccaagtctttcttcagttccaaggccaaggtgatttatctcatgagaaatcccagagatgttttggtgtctggttattttttctggaaaaacatgaagtttattaagaaaccaaagtcatgggaagaatattttgaatggttttgtcaaggaactgtgctatatgggtcatggtttgaccacattcatggctggatgcccatgagagaggagaaaaacttcctgttactgagttatgaggagctgaaacaggacacaggaagaaccatagagaagatctgtcaattcctgggaaagacgttagaacccgaagaactgaacttaattctcaagaacagctcctttcagagcatgaaagaaaacaagatgtccaattattccctcctgagtgttgattatgtagtggacaaagcacaacttctgagaaaaggtgtatctggggactggaaaaatcacttcacagtggcccaagctgaagactttgataaattgttccaagagaagatggcagatcttcctcgagagctgttcccatgggaataacgtccaaaacactctggatcttatatggagaatgacattgattctcctgtccttgtacatgtacctgactggggtcattgtgtaagacttattattttatcctgaaaccttaaatatcaaacctctgcatctctgatcccttccttgttaaaagttaccacggttggccaggcgcggtggttcatgcctgtaatcccagcactatgggaggccgagacgggcggatcacgaggtcaggagactgagaccatcctggctaacacggtgaaaccccatctctactaaaaatacaaaaaacaaaaaaaattagccaggcgcattggctcatgtctgtaatcccagcactttgggaggtcgggggggtgggggaggatcacggggtcaggagatcgagaccatcctggccaacatgatgaaaccctatctctactaaaaatacaaaaattagccgggcatggtggtgcacgcctatagtcccagctactcgggaggctgaggtaggagaatcgtttgaactcaggaggcagaggttgcaatgagccaagatcgcgccactgcactccagcctgggtgacagagcgagaccgtctcaaaaagaaagaagtgactagggttcagagaaccagggttcaaagcccagggatgcaaaggttgcagtgagttgagtcatgggatcccagacttttttaaatgtttgcaatgtttcccgtttacagaatgctacaagaataatgtacgtactacctaaaaggatgtctaaatgtttgttaataaaaataagaaatagctacagtgacagattttagagcaaaaattagtaataaaaataagaaataaaatta

SEQ ID NO: 12 - protein, 285 aa, Homo sapiens
MSDDFLWFEGIAFPTMGFRSETLRKVRDEFVIRDEDVIILTYPKSGTNWLAEILCLMHSKGDAKWIQSVPIWERSPWVESEIGYTALSETESPRLFSSHLPIQLFPKSFFSSKAKVIYLMRNPRDVLVSGYFFWKNMKFIKKPKSWEEYFEWFCQGTVLYGSWFDHIHGWMPMREEKNFLLLSYEELKQDTGRTIEKICQFLGKTLEPEELNLILKNSSFQSMKENKMSNYSLLSVDYVVDKAQLLRKGVSGDWKNHFTVAQAEDFDKLFQEKMADLPRELFPWE

SEQ ID NO: 13 - DNA, 1484 bp, Homo sapiens
atggcggcggtgtctggcttggtgcggagaccccttcgggaggtctccgggctgctgaagaggcgctttcactggaccgcgccggctgcgctgcaggtgacagttcgtgatgctataaatcagggtatggatgaggagctggaaagagatgagaaggtatttctgcttggagaagaagttgcccagtatgatggggcatacaaggttagtcgagggctgtggaagaaatatggagacaagaggattattgacactcccatatcagagatgggctttgctggaattgctgtaggtgcagctatggctgggttgcggcccatttgtgaatttatgaccttcaatttctccatgcaagccattgaccaggttataaactcagctgccaagacctactacatgtctggtggccttcagcctgtgcctatagtcttcaggggacccaatggtgcctcagcaggtgtagctgcccagcactcacagtgctttgctgcctggtatgggcactgcccaggcttaaaggtggtcagtccctggaattcagaggatgctaaaggacttattaaatcagccattcgggataacaatccagtggtggtgctagagaatgaattgatgtatggggttccttttgaatttcctccggaagctcagtcaaaagattttctgattcctattggaaaagccaaaatagaaaggcaaggaacacatataactgtggtttcccattcaagacctgtgggccactgcttagaagctgcagcagtgctatctaaagaaggagttgaatgtgaggtgataaatatgcgtaccattagaccaatggacatggaaaccatagaagccagtgtcatgaagacaaatcatcttgtaactgtggaaggaggctggccacagtttggagtaggagctgaaatctgtgccaggatcatggaaggtcctgcgttcaatttcctggatgctcctgctgttcgtgtcactggtgctgatgtccctatgccttatgcaaagattctagaggacaactctatacctcaggtcaaagacatcatatttgcaataaagaaaacattaaatatttagtttggacttgaatatcaagtcgttgaaatttatttgaaatacttgctggcactgcacctggatttgtactgcaagacctgactattcataaaggaaaacgatttctaaagcaacagcaggtatttttgtacagggaagtttaaatgtgtttgtgtatggaaaactctccactctcctcccctagatgccatgcttccttttgtctgttacggttgccatgttctttgaataacaaattatatcacattttatcctctctcaccacaaggacaaagtatggatgtggcagagtcctgatgaaagatgtatccaaacaagataacttatatgtataaaattaaagcatataatacacatttactgttagtttgttttgataaggaataaaggaatttctaacatga

SEQ ID NO: 14 - protein, 359 aa, Homo sapiens [sequence omitted]

SEQ ID NO: 15 - DNA, 1653 bp, Homo sapiens
cggttgcttgctgtcccagcggcgccccctcatcaccgtcgccatgcccggaggtctgcttctcggggacgtggctcccaactttgaggccaataccaccgtcggccgcatccgtttccacgactttctgggagactcatggggcattctcttctcccaccctcgggactttaccccagtgtgcaccacagagcttggcagagctgcaaagctggcaccagaatttgccaagaggaatgttaagttgattgccctttcaatagacagtgttgaggaccatcttgcctggagcaaggatatcaatgcttacaattgtgaagagcccacagaaaagttaccttttcccatcatcgatgataggaatcgggagcttgccatcctgttgggcatgctggatccagcagagaaggatgaaaagggcatgcctgtgacagctcgtgtggtgtttgtttttggtcctgataagaagctgaagctgtctatcctctacccagctaccactggcaggaactttgatgagattctcagggtagtcatctctctccagctgacagcagaaaaaagggttgccaccccagttgattggaaggatggggatagtgtgatggtccttccaaccatccctgaagaagaagccaaaaaacttttcccgaaaggagtcttcaccaaagagctcccatctggcaagaaatacctccgctacacaccccagccttaagtctcttggagaagttggtgctgtgagccagaggatgtcagctgccaattgtgttttcctgcagcaattccataaacacatcctggtgtcatcacagccaaggtttttaggttgctataccaatggcttattaaatgaaaatggcactaaaagtttcttgagattctttatactctctgccttcagcaatcaattccattcatacatcagcactctgctggttctgtttgaaatatgttctgtatttaaaactcaaatcttgttggatctctgcagggcttgtgaccaatgaagtcatatttgttgatggttgacaaagcttgcttcactccatcagagaatgactatcaatttttttttaactgtcctatcacgtcctctcctgtcacccattttgaagagtggcagaacttgaagttcaacttcctctgtaaatatccaagtataaagcccaggaacttctagaataacccagatgcgctttaattttttttaatatgttttgatcacagaacttctagaataacccagatgctctttcatattcttttaatacatcttgatcacagctgggggaaaaaaagctttttaattctgtaccttcctagtagataagtgaagagcagggaaagagacctttaaatattttgctataaaaaaatttgtgataagtttctatcaaaatggggagattgcagaaaaggcttcccttggctcccaaggaggtgtagcaggtgtgagcaatattagtgccatgtgcctttcacacagggtttgcatttatcagtctgttttccgatgatgtgtacatgaaagagtacaccatgtgaagagaagagagaatgattgaaaatgttttagtatagaactcttcttgcagtgggttgctattttctagattttactttttagggaacaaaataaaatcctttgtt

SEQ ID NO: 16 - protein, 224 aa, Homo sapiens
MPGGLLLGDVAPNFEANTTVGRIRFHDFLGDSWGILFSHPRDFTPVCTTELGRAAKLAPEFAKRNVKLIALSIDSVEDHLAWSKDINAYNCEEPTEKLPFPIIDDRNRELAILLGMLDPAEKDEKGMPVTARVVFVFGPDKKLKLSILYPATTGRNFDEILRVVISLQLTAEKRVATPVDWKDGDSVMVLPTIPEEEAKKLFPKGVFTKELPSGKKYLRYTPQP

SEQ ID NO: 17 - DNA, 858 bp, Homo sapiens
atggccgggctcggccaccccgccgccttcggccgggccacccacgccgtggtgcgggcgctacccgagtcgctcggccagcacgcgctgagaagcgccaagggcgaggaggtggacgtcgcccgcgcggaacggcagcaccagctctacgtgggcgtgctgggcagcaagctggggctgcaggtggtggagctgccggccgacgagagccttccggactgcgtcttcgtggaggacgtggccgtggtgtgcgaggagacggccctcatcacccgacccggggcgccgagccggaggaaggaggttgacatgatgaaagaagcattagaaaaacttcagctcaatatagtagagatgaaagatgaaaatgcaactttagatggcggagatgttttattcacaggcagagaattttttgtgggcctttccaaaaggacaaatcaacgaggtgctgaaatcttggctgatacttttaaggactatgcagtctccacagtgccagtggcagatgggttgcatttgaagagtttctgcagcatggctgggcctaacctgatcgcaattgggtctagtgaatctgcacagaaggcccttaagatcatgcaacagatgagtgaccaccgctacgacaaactcactgtgcctgatgacatagcagcaaactgtatatatctaaatatccccaacaaagggcacgtcttgctgcaccgaaccccggaagagtatccagaaagtgcaaaggtttatgagaaactgaaggaccatatgctgatccccgtgagcatgtctgaactggaaaaggtggatgggctgctcacctgctgctcagttttaattaacaagaaagtagactcctga

SEQ ID NO: 18 - protein, 285 aa, Homo sapiens
MAGLGHPAAFGRATHAVVRALPESLGQHALRSAKGEEVDVARAERQHQLYVGVLGSKLGLQVVELPADESLPDCVFVEDVAVVCEETALITRPGAPSRRKEVDMMKEALEKLQLNIVEMKDENATLDGGDVLFTGREFFVGLSKRTNQRGAEILADTFKDYAVSTVPVADGLHLKSFCSMAGPNLIAIGSSESAQKALKIMQQMSDHRYDKLTVPDDIAANCIYLNIPNKGHVLLHRTPEEYPESAKVYEKLKDHMLIPVSMSELEKVDGLLTCCSVLINKKVDS